(19) United States
(12) Patent Application Publication
(10) Pub. No.: US 2026/0201041 A1
(43) Pub. Date: Jul. 16, 2026

(54) ANTI-CD103 ANTIBODIES

(71) Applicants: IMMIOS HOLDING B.V., OSS (NL); RIJKSUNIVERSITEIT GRONINGEN, Groningen (NL); ACADEMISCH ZIEKENHUIS GRONINGEN, Groningen (NL)

(72) Inventors: Marco DE BRUYN, Haren (NL); Klaas Jan-Derek KOL, Groningen (NL); Hans Wilhelm NIJMAN, Groningen (NL); Hans VAN EENENNAAM, Velp (NL); Sander Martinus Johannes VAN DUIJNHOVEN, Uden (NL)

(73) Assignees: IMMIOS HOLDING B.V., OSS (NL); RIJKSUNIVERSITEIT GRONINGEN, Groningen (NL); ACADEMISCH ZIEKENHUIS GRONINGEN, Groningen (NL)

(21) Appl. No.: 19/390,252

(22) Filed: Nov. 14, 2025

Related U.S. Application Data
(63) Continuation of application No. 17/997,240, filed on Apr. 30, 2021, now Pat. No. 12,545,733.
(60) Provisional application No. 62/704,258, filed on Apr. 30, 2020.

(51) Int. Cl. C07K 16/28; A61K 51/10; G01N 33/533

(52) U.S. Cl. CPC C07K 16/2839; A61K 51/1027; A61K 51/1093; G01N 33/533; C07K 2317/24; C07K 2317/52; C07K 2317/565

(57) ABSTRACT

The present invention relates to anti-CD103 antibodies, as well as use of these antibodies in diagnosis, prognosis, monitoring, and treatment of diseases.

### RELATED APPLICATION

The present invention is a continuation of U.S. patent application Ser. No. 17/997,240, which was filed under 35 U.S.C. § 371 as the U.S. national phase of International Application No. PCT/EP2021/061450, filed Apr. 30, 2021, which designated the United States and claims priority to U.S. Provisional Application No. 62/704,258, filed Apr. 30, 2020, each of which is hereby incorporated by reference in its entirety.

### REFERENCE TO ELECTRONIC SEQUENCE LISTING

The application contains a Sequence Listing which has been submitted electronically in .XML format and is hereby incorporated by reference in its entirety. Said .XML copy, created on Dec. 12, 2025, is named “IMMIOS-0010-CT1.xml” and is 59,306 bytes in size. The sequence listing contained in this .XML file is part of the specification and is hereby incorporated by reference herein in its entirety.

### FIELD OF THE INVENTION

The present invention relates to anti-CD103 antibodies, as well as use of these antibodies in the diagnosis, prognosis, monitoring, and treatment of diseases.

### BACKGROUND OF THE INVENTION

CD103 (Integrin αE) is type I membrane protein expressed on a subpopulation of lamina propria T cells, epithelial dendritic cells, lamina propria-derived dendritic cells, and a small subset of peripheral lymphocytes. Treg cells express a high level of CD103. Mature CD103 protein can be cleaved into 2 chains, a 150 kD (C-terminal) chain and a 25 kD (N-terminal) chain, which remain linked by disulfide bonds. In combination with the β7 integrin, CD103 forms an aE/B7 heterodimer, which represents the E-cadherin binding integrin known as the human mucosal lymphocyte-1 antigen.

The identification and understanding of immune checkpoint proteins and their role in the immune response represents a breakthrough in cancer therapy. With this discovery, efforts focussed on blocking the immune checkpoint pathways in an attempt to activate T-cells directed at cancer cells that were found to be ineffective in stimulating a productive antitumor response. Ipilimumab, an antibody which binds to and functionally blocks CTLA-4, was approved by the US Food and Drug Administration (FDA) for the treatment of metastatic melanoma in 2011. Following on the heels of ipilimumab, antibodies targeting the programmed cell death-1 (PD-1) receptor, and its ligand found on many cancer cells programmed death-ligand 1 (PD-L1), were also approved. These checkpoint inhibitors have led to a revolution in cancer therapy.

Despite significant clinical benefit ascribed to immunotherapy targeting immune checkpoint pathways, a majority of cancer patients fail to respond to checkpoint inhibitors. In particular, studies suggest that checkpoint inhibition may not be sufficient in patients that display limited functional T cell infiltration into the tumor environment. Moreover, while T cells may accumulate in tissues surrounding the tumor mass, they may not be interacting with tumor cells themselves.

Through their ability to produce T cell-specific chemokines and present antigens together with costimulatory or inhibitory signals, tumor-associated antigen presenting cells are best poised to shape anti-tumoral effector immunity. Tissue-resident dendritic cells consist of two functionally specialized subsets: the CD103⁺-CD8⁺ DCs that participate in priming and cross-presentation of cell-associated antigens to CD8⁺ T cells, and CD11b⁺ DCs that are more potent at driving CD4⁺ helper T cell responses. Type I interferon production by the CD103⁺-CD8⁺ DC lineage controls spontaneous T cell priming to tumor antigens. Thus, the composition of the tumor-associated myeloid compartment likely plays a key role in tumor response to checkpoint blockade.

The reported association of CD103⁺ tumor-infiltrating T lymphocytes with improved clinical outcomes in cancer patients highlights the need to have a clear and comprehensive understanding of the expression characteristics of cancer-specific lymphocytes and their implications for future immunotherapies.

### SUMMARY OF THE INVENTION

In a first aspect, the invention provides anti-CD103 antibodies and antigen binding fragments thereof comprising the structural and functional features specified below.

In various embodiments, the invention provides an antibody or antigen binding fragment thereof that binds to human CD103 comprising:
a. a heavy chain variable region CDR1 comprising the amino acid sequence of SEQ ID NO: 1 or an amino acid sequence differing from SEQ ID NO: 1 by 1, 2, or 3 conservative substitutions,
b. a heavy chain variable region CDR2 comprising the amino acid sequence of SEQ ID NO: 2 or an amino acid sequence differing from SEQ ID NO: 2 by 1, 2, or 3 conservative substitutions,
c. a heavy chain variable region CDR3 comprising the amino acid sequence of SEQ ID NO: 3 or an amino acid sequence differing from SEQ ID NO: 3 by 1, 2, or 3 conservative substitutions,
d. a light chain variable region CDR1 comprising the amino acid sequence of SEQ ID NO: 4 or an amino acid sequence differing from SEQ ID NO: 4 by 1, 2, or 3 conservative substitutions,
e. a light chain variable region CDR2 comprising the amino acid sequence of SEQ ID NO: 5 or an amino acid sequence differing from SEQ ID NO: 5 by 1, 2, or 3 conservative substitutions, and
f. a light chain variable region CDR3 comprising the amino acid sequence of SEQ ID NO: 6 or an amino acid sequence differing from SEQ ID NO: 6 by 1, 2, or 3 conservative substitutions;
or
g. a heavy chain variable region CDR1 comprising the amino acid sequence of SEQ ID NO: 9 or an amino acid sequence differing from SEQ ID NO: 9 by 1, 2, or 3 conservative substitutions,
h. a heavy chain variable region CDR2 comprising the amino acid sequence of SEQ ID NO: 10 or an amino acid sequence differing from SEQ ID NO: 10 by 1, 2, or 3 conservative substitutions,
i. a heavy chain variable region CDR3 comprising the amino acid sequence of SEQ ID NO: 11 or an amino acid sequence differing from SEQ ID NO: 11 by 1, 2, or 3 conservative substitutions,
j. a light chain variable region CDR1 comprising the amino acid sequence of SEQ ID NO: 12 or an amino acid sequence differing from SEQ ID NO: 12 by 1, 2, or 3 conservative substitutions,
k. a light chain variable region CDR2 comprising the amino acid sequence of SEQ ID NO: 13 or an amino acid sequence differing from SEQ ID NO: 13 by 1, 2, or 3 conservative substitutions, and
l. a light chain variable region CDR3 comprising the amino acid sequence of SEQ ID NO: 14 or an amino acid sequence differing from SEQ ID NO: 14 by 1, 2, or 3 conservative substitutions;
or
m. a heavy chain variable region CDR1 comprising the amino acid sequence of SEQ ID NO: 17 or an amino acid sequence differing from SEQ ID NO: 17 by 1, 2, or 3 conservative substitutions,
n. a heavy chain variable region CDR2 comprising the amino acid sequence of SEQ ID NO: 18 or an amino acid sequence differing from SEQ ID NO: 18 by 1, 2, or 3 conservative substitutions,
o. a heavy chain variable region CDR3 comprising the amino acid sequence of SEQ ID NO: 19 or an amino acid sequence differing from SEQ ID NO: 19 by
p. a light chain variable region CDR1 comprising the amino acid sequence of SEQ ID NO: 20 or an amino acid sequence differing from SEQ ID NO: 20 by 1, 2, or 3 conservative substitutions,
q. a light chain variable region CDR2 comprising the amino acid sequence of SEQ ID NO: 21 or an amino acid sequence differing from SEQ ID NO: 21 by 1, 2, or 3 conservative substitutions, and
r. a light chain variable region CDR3 comprising the amino acid sequence of SEQ ID NO: 22 or an amino acid sequence differing from SEQ ID NO: 22 by 1, 2, or 3 conservative substitutions;
or
s. a heavy chain variable region CDR1 comprising the amino acid sequence of SEQ ID NO: 25 or an amino acid sequence differing from SEQ ID NO: 25 by 1, 2, or 3 conservative substitutions,
t. a heavy chain variable region CDR2 comprising the amino acid sequence of SEQ ID NO: 26 or an amino acid sequence differing from SEQ ID NO: 26 by 1, 2, or 3 conservative substitutions,
u. a heavy chain variable region CDR3 comprising the amino acid sequence of SEQ ID NO: 27 or an amino acid sequence differing from SEQ ID NO: 27 by 1, 2, or 3 conservative substitutions,
v. a light chain variable region CDR1 comprising the amino acid sequence of SEQ ID NO: 28 or an amino acid sequence differing from SEQ ID NO: 28 by 1, 2, or 3 conservative substitutions,
w. a light chain variable region CDR2 comprising the amino acid sequence of SEQ ID NO: 29 or an amino acid sequence differing from SEQ ID NO: 29 by 1, 2, or 3 conservative substitutions, and
x. a light chain variable region CDR3 comprising the amino acid sequence of SEQ ID NO: 30 or an amino acid sequence differing from SEQ ID NO: 30 by 1, 2, or 3 conservative substitutions;
or
y. a heavy chain variable region CDR1 comprising the amino acid sequence of SEQ ID NO: 33 or an amino acid sequence differing from SEQ ID NO: 33 by 1, 2, or 3 conservative substitutions,
z. a heavy chain variable region CDR2 comprising the amino acid sequence of SEQ ID NO: 34 or an amino acid sequence differing from SEQ ID NO: 34 by 1, 2, or 3 conservative substitutions,
aa. a heavy chain variable region CDR3 comprising the amino acid sequence of SEQ ID NO: 35 or an amino acid sequence differing from SEQ ID NO: 35 by 1, 2, or 3 conservative substitutions,
bb. a light chain variable region CDR1 comprising the amino acid sequence of SEQ ID NO: 36 or an amino acid sequence differing from SEQ ID NO: 36 by 1, 2, or 3 conservative substitutions,
cc. a light chain variable region CDR2 comprising the amino acid sequence of SEQ ID NO: 37 or an amino acid sequence differing from SEQ ID NO: 37 by 1, 2, or 3 conservative substitutions, and
dd. a light chain variable region CDR3 comprising the amino acid sequence of SEQ ID NO: 38 or an amino acid sequence differing from SEQ ID NO: 38 by 1, 2, or 3 conservative substitutions.

In one embodiment, the invention provides an antibody that binds to human CD103 comprising:
a. a heavy chain variable region CDR1 comprising the amino acid sequence of SEQ ID NO: 1,
b. a heavy chain variable region CDR2 comprising the amino acid sequence of SEQ ID NO: 2,
c. a heavy chain variable region CDR3 comprising the amino acid sequence of SEQ ID NO: 3,
d. a light chain variable region CDR1 comprising the amino acid sequence of SEQ ID NO: 4,
e. a light chain variable region CDR2 comprising the amino acid sequence of SEQ ID NO: 5, and
f. a light chain variable region CDR3 comprising the amino acid sequence of SEQ ID NO: 6.

In one embodiment, the invention provides an antibody that binds to human CD103 comprising:
a. a heavy chain variable region CDR1 comprising the amino acid sequence of SEQ ID NO: 9,
b. a heavy chain variable region CDR2 comprising the amino acid sequence of SEQ ID NO: 10,
c. a heavy chain variable region CDR3 comprising the amino acid sequence of SEQ ID NO: 11,
d. a light chain variable region CDR1 comprising the amino acid sequence of SEQ ID NO: 12,
e. a light chain variable region CDR2 comprising the amino acid sequence of SEQ ID NO: 13, and
f. a light chain variable region CDR3 comprising the amino acid sequence of SEQ ID NO: 14.

In one embodiment, the invention provides an antibody that binds to human CD103 comprising:
a. a heavy chain variable region CDR1 comprising the amino acid sequence of SEQ ID NO: 17,
b. a heavy chain variable region CDR2 comprising the amino acid sequence of SEQ ID NO: 18,
c. a heavy chain variable region CDR3 comprising the amino acid sequence of SEQ ID NO: 19,
d. a light chain variable region CDR1 comprising the amino acid sequence of SEQ ID NO: 20,
e. a light chain variable region CDR2 comprising the amino acid sequence of SEQ ID NO: 21, and
f. a light chain variable region CDR3 comprising the amino acid sequence of SEQ ID NO: 22.

In one embodiment, the invention provides an antibody that binds to human CD103 comprising:
a. a heavy chain variable region CDR1 comprising the amino acid sequence of SEQ ID NO: 25,
b. a heavy chain variable region CDR2 comprising the amino acid sequence of SEQ ID NO: 26,
c. a heavy chain variable region CDR3 comprising the amino acid sequence of SEQ ID NO: 27,
d. a light chain variable region CDR1 comprising the amino acid sequence of SEQ ID NO: 28,
e. a light chain variable region CDR2 comprising the amino acid sequence of SEQ ID NO: 29, and
f. a light chain variable region CDR3 comprising the amino acid sequence of SEQ ID NO: 30.

In one embodiment, the invention provides an antibody that binds to human CD103 comprising:
a. a heavy chain variable region CDR1 comprising the amino acid sequence of SEQ ID NO: 33,
b. a heavy chain variable region CDR2 comprising the amino acid sequence of SEQ ID NO: 34,
c. a heavy chain variable region CDR3 comprising the amino acid sequence of SEQ ID NO: 35,
d. a light chain variable region CDR1 comprising the amino acid sequence of SEQ ID NO: 36,
e. a light chain variable region CDR2 comprising the amino acid sequence of SEQ ID NO: 37, and
f. a light chain variable region CDR3 comprising the amino acid sequence of SEQ ID NO: 38.

In one embodiment, the invention provides an antibody that binds to human CD103 comprising:
a. a heavy chain variable region CDR1 comprising an amino acid sequence differing from SEQ ID NO: 1 by 1 conservative substitution,
b. a heavy chain variable region CDR2 comprising an amino acid sequence differing from SEQ ID NO: 2 by 1 conservative substitution,
c. a heavy chain variable region CDR3 comprising an amino acid sequence differing from SEQ ID NO: 3 by 1 conservative substitution,
d. a light chain variable region CDR1 comprising an amino acid sequence differing from SEQ ID NO: 4 by 1 conservative substitution,
e. a light chain variable region CDR2 comprising an amino acid sequence differing from SEQ ID NO: 5 by 1 conservative substitution, and
f. a light chain variable region CDR3 comprising an amino acid sequence differing from SEQ ID NO: 6 by 1 conservative substitution.

In one embodiment, the invention provides an antibody that binds to human CD103 comprising:
a. a heavy chain variable region CDR1 comprising an amino acid sequence differing from SEQ ID NO: 9 by 1 conservative substitution,
b. a heavy chain variable region CDR2 comprising an amino acid sequence differing from SEQ ID NO: 10 by 1 conservative substitution,
c. a heavy chain variable region CDR3 comprising an amino acid sequence differing from SEQ ID NO: 11 by 1 conservative substitution,
d. a light chain variable region CDR1 comprising an amino acid sequence differing from SEQ ID NO: 12 by 1 conservative substitution,
e. a light chain variable region CDR2 comprising an amino acid sequence differing from SEQ ID NO: 13 by 1 conservative substitution, and
f. a light chain variable region CDR3 comprising an amino acid sequence differing from SEQ ID NO: 14 by 1 conservative substitution.

In one embodiment, the invention provides an antibody that binds to human CD103 comprising:
a. a heavy chain variable region CDR1 comprising an amino acid sequence differing from SEQ ID NO: 17 by 1 conservative substitution,
b. a heavy chain variable region CDR2 comprising an amino acid sequence differing from SEQ ID NO: 18 by 1 conservative substitution,
c. a heavy chain variable region CDR3 comprising an amino acid sequence differing from SEQ ID NO: 19 by 1 conservative substitution,
d. a light chain variable region CDR1 comprising an amino acid sequence differing from SEQ ID NO: 20 by 1 conservative substitution,
e. a light chain variable region CDR2 comprising an amino acid sequence differing from SEQ ID NO: 21 by 1 conservative substitution, and
f. a light chain variable region CDR3 comprising an amino acid sequence differing from SEQ ID NO: 22 by 1 conservative substitution.

In one embodiment, the invention provides an antibody that binds to human CD103 comprising:
a. a heavy chain variable region CDR1 comprising an amino acid sequence differing from SEQ ID NO: 25 by 1 conservative substitution,
b. a heavy chain variable region CDR2 comprising an amino acid sequence differing from SEQ ID NO: 26 by 1 conservative substitution,
c. a heavy chain variable region CDR3 comprising an amino acid sequence differing from SEQ ID NO: 27 by 1 conservative substitution,
d. a light chain variable region CDR1 comprising an amino acid sequence differing from SEQ ID NO: 28 by 1 conservative substitution,
e. a light chain variable region CDR2 comprising an amino acid sequence differing from SEQ ID NO: 29 by 1 conservative substitution, and
f. a light chain variable region CDR3 comprising an amino acid sequence differing from SEQ ID NO: 30 by 1 conservative substitution.

In one embodiment, the invention provides an antibody that binds to human CD103 comprising:
a. a heavy chain variable region CDR1 comprising an amino acid sequence differing from SEQ ID NO: 33 by 1 conservative substitution,
b. a heavy chain variable region CDR2 comprising an amino acid sequence differing from SEQ ID NO: 34 by 1 conservative substitution,
c. a heavy chain variable region CDR3 comprising an amino acid sequence differing from SEQ ID NO: 35 by 1 conservative substitution,
d. a light chain variable region CDR1 comprising an amino acid sequence differing from SEQ ID NO: 36 by 1 conservative substitution,
e. a light chain variable region CDR2 comprising an amino acid sequence differing from SEQ ID NO: 37 by 1 conservative substitution, and
f. a light chain variable region CDR3 comprising an amino acid sequence differing from SEQ ID NO: 38 by 1 conservative substitution.

In one embodiment, the invention provides an antibody that binds to human CD103 comprising:
a. a heavy chain variable region CDR1 comprising an amino acid sequence differing from SEQ ID NO: 1 by 2 conservative substitutions,
b. a heavy chain variable region CDR2 comprising an amino acid sequence differing from SEQ ID NO: 2 by 2 conservative substitutions,
c. a heavy chain variable region CDR3 comprising an amino acid sequence differing from SEQ ID NO: 3 by 2 conservative substitutions,
d. a light chain variable region CDR1 comprising an amino acid sequence differing from SEQ ID NO: 4 by 2 conservative substitutions,
e. a light chain variable region CDR2 comprising an amino acid sequence differing from SEQ ID NO: 5 by 2 conservative substitutions, and
f. a light chain variable region CDR3 comprising an amino acid sequence differing from SEQ ID NO: 6 by 2 conservative substitutions.

In one embodiment, the invention provides an antibody that binds to human CD103 comprising:
a. a heavy chain variable region CDR1 comprising an amino acid sequence differing from SEQ ID NO: 9 by 2 conservative substitutions,
b. a heavy chain variable region CDR2 comprising an amino acid sequence differing from SEQ ID NO: 10 by 2 conservative substitutions,
c. a heavy chain variable region CDR3 comprising an amino acid sequence differing from SEQ ID NO: 11 by 2 conservative substitutions,
d. a light chain variable region CDR1 comprising an amino acid sequence differing from SEQ ID NO: 12 by 2 conservative substitutions,
e. a light chain variable region CDR2 comprising an amino acid sequence differing from SEQ ID NO: 13 by 2 conservative substitutions, and
f. a light chain variable region CDR3 comprising an amino acid sequence differing from SEQ ID NO: 14 by 2 conservative substitutions.

In one embodiment, the invention provides an antibody that binds to human CD103 comprising:
a. a heavy chain variable region CDR1 comprising an amino acid sequence differing from SEQ ID NO: 17 by 2 conservative substitutions,
b. a heavy chain variable region CDR2 comprising an amino acid sequence differing from SEQ ID NO: 18 by 2 conservative substitutions,
c. a heavy chain variable region CDR3 comprising an amino acid sequence differing from SEQ ID NO: 19 by 2 conservative substitutions,
d. a light chain variable region CDR1 comprising an amino acid sequence differing from SEQ ID NO: 20 by 2 conservative substitutions,
e. a light chain variable region CDR2 comprising an amino acid sequence differing from SEQ ID NO: 21 by 2 conservative substitutions, and
f. a light chain variable region CDR3 comprising an amino acid sequence differing from SEQ ID NO: 22 by 2 conservative substitutions.

In one embodiment, the invention provides an antibody that binds to human CD103 comprising:
a. a heavy chain variable region CDR1 comprising an amino acid sequence differing from SEQ ID NO: 25 by 2 conservative substitutions,
b. a heavy chain variable region CDR2 comprising an amino acid sequence differing from SEQ ID NO: 26 by 2 conservative substitutions,
c. a heavy chain variable region CDR3 comprising an amino acid sequence differing from SEQ ID NO: 27 by 2 conservative substitutions,
d. a light chain variable region CDR1 comprising an amino acid sequence differing from SEQ ID NO: 28 by 2 conservative substitutions,
e. a light chain variable region CDR2 comprising an amino acid sequence differing from SEQ ID NO: 29 by 2 conservative substitutions, and
f. a light chain variable region CDR3 comprising an amino acid sequence differing from SEQ ID NO: 30 by 2 conservative substitutions.

In one embodiment, the invention provides an antibody that binds to human CD103 comprising:
a. a heavy chain variable region CDR1 comprising an amino acid sequence differing from SEQ ID NO: 33 by 2 conservative substitutions,
b. a heavy chain variable region CDR2 comprising an amino acid sequence differing from SEQ ID NO: 34 by 2 conservative substitutions,
c. a heavy chain variable region CDR3 comprising an amino acid sequence differing from SEQ ID NO: 35 by 2 conservative substitutions,
d. a light chain variable region CDR1 comprising an amino acid sequence differing from SEQ ID NO: 36 by 2 conservative substitutions,
e. a light chain variable region CDR2 comprising an amino acid sequence differing from SEQ ID NO: 37 by 2 conservative substitutions, and
f. a light chain variable region CDR3 comprising an amino acid sequence differing from SEQ ID NO: 38 by 2 conservative substitutions.

In one embodiment, the invention provides an antibody that binds to human CD103 comprising:
a. a heavy chain variable region CDR1 comprising an amino acid sequence differing from SEQ ID NO: 1 by 3 conservative substitutions,
b. a heavy chain variable region CDR2 comprising an amino acid sequence differing from SEQ ID NO: 2 by 3 conservative substitutions,
c. a heavy chain variable region CDR3 comprising an amino acid sequence differing from SEQ ID NO: 3 by 3 conservative substitutions,
d. a light chain variable region CDR1 comprising an amino acid sequence differing from SEQ ID NO: 4 by 3 conservative substitutions,
e. a light chain variable region CDR2 comprising an amino acid sequence differing from SEQ ID NO: 5 by 3 conservative substitutions, and
f. a light chain variable region CDR3 comprising an amino acid sequence differing from SEQ ID NO: 6 by 3 conservative substitutions.

In one embodiment, the invention provides an antibody that binds to human CD103 comprising:
a. a heavy chain variable region CDR1 comprising an amino acid sequence differing from SEQ ID NO: 9 by 3 conservative substitutions,
b. a heavy chain variable region CDR2 comprising an amino acid sequence differing from SEQ ID NO: 10 by 3 conservative substitutions,
c. a heavy chain variable region CDR3 comprising an amino acid sequence differing from SEQ ID NO: 11 by 3 conservative substitutions,
d. a light chain variable region CDR1 comprising an amino acid sequence differing from SEQ ID NO: 12 by 3 conservative substitutions,
e. a light chain variable region CDR2 comprising an amino acid sequence differing from SEQ ID NO: 13 by 3 conservative substitutions, and
f. a light chain variable region CDR3 comprising an amino acid sequence differing from SEQ ID NO: 14 by 3 conservative substitutions.

In one embodiment, the invention provides an antibody that binds to human CD103 comprising:
a. a heavy chain variable region CDR1 comprising an amino acid sequence differing from SEQ ID NO: 17 by 3 conservative substitutions,
b. a heavy chain variable region CDR2 comprising an amino acid sequence differing from SEQ ID NO: 18 by 3 conservative substitutions,
c. a heavy chain variable region CDR3 comprising an amino acid sequence differing from SEQ ID NO: 19 by 3 conservative substitutions,
d. a light chain variable region CDR1 comprising an amino acid sequence differing from SEQ ID NO: 20 by 3 conservative substitutions,
e. a light chain variable region CDR2 comprising an amino acid sequence differing from SEQ ID NO: 21 by 3 conservative substitutions, and
f. a light chain variable region CDR3 comprising an amino acid sequence differing from SEQ ID NO: 22 by 3 conservative substitutions.

In one embodiment, the invention provides an antibody that binds to human CD103 comprising:
a. a heavy chain variable region CDR1 comprising an amino acid sequence differing from SEQ ID NO: 25 by 3 conservative substitutions,
b. a heavy chain variable region CDR2 comprising an amino acid sequence differing from SEQ ID NO: 26 by 3 conservative substitutions,
c. a heavy chain variable region CDR3 comprising an amino acid sequence differing from SEQ ID NO: 27 by 3 conservative substitutions,
d. a light chain variable region CDR1 comprising an amino acid sequence differing from SEQ ID NO: 28 by 3 conservative substitutions,
e. a light chain variable region CDR2 comprising an amino acid sequence differing from SEQ ID NO: 29 by 3 conservative substitutions, and
f. a light chain variable region CDR3 comprising an amino acid sequence differing from SEQ ID NO: 30 by 3 conservative substitutions.

In one embodiment, the invention provides an antibody that binds to human CD103 comprising:
a. a heavy chain variable region CDR1 comprising an amino acid sequence differing from SEQ ID NO: 33 by 3 conservative substitutions,
b. a heavy chain variable region CDR2 comprising an amino acid sequence differing from SEQ ID NO: 34 by 3 conservative substitutions,
c. a heavy chain variable region CDR3 comprising an amino acid sequence differing from SEQ ID NO: 35 by 3 conservative substitutions,
d. a light chain variable region CDR1 comprising an amino acid sequence differing from SEQ ID NO: 36 by 3 conservative substitutions,
e. a light chain variable region CDR2 comprising an amino acid sequence differing from SEQ ID NO: 37 by 3 conservative substitutions, and
f. a light chain variable region CDR3 comprising an amino acid sequence differing from SEQ ID NO: 38 by 3 conservative substitutions.

In various other embodiments, the invention provides an antibody or antigen binding fragment thereof that binds to human CD103 comprising:
a. a heavy chain of the antibody or antigen binding fragment that comprises the amino acid sequence of SEQ ID NO: 7 and a light chain of the antibody or antigen binding fragment that comprises the amino acid sequence of SEQ ID NO: 8; or
b. a heavy chain of the antibody or antigen binding fragment that comprises the amino acid sequence of SEQ ID NO: 15 and a light chain of the antibody or antigen binding fragment that comprises the amino acid sequence of SEQ ID NO: 16; or
c. a heavy chain of the antibody or antigen binding fragment that comprises the amino acid sequence of SEQ ID NO: 23 and a light chain of the antibody or antigen binding fragment that comprises the amino acid sequence of SEQ ID NO: 24; or
d. a heavy chain of the antibody or antigen binding fragment that comprises the amino acid sequence of SEQ ID NO: 31 and a light chain of the antibody or antigen binding fragment that comprises the amino acid sequence of SEQ ID NO: 32; or
e. a heavy chain of the antibody or antigen binding fragment that comprises the amino acid sequence of SEQ ID NO: 39 and a light chain of the antibody or antigen binding fragment that comprises the amino acid sequence of SEQ ID NO: 40;
or in each case a sequence having at least 95% (and more preferably 97% or 99%) sequence similarity or identity to a given SEQ ID NO, with any difference in such sequence being confined to amino acids that are not a part of a CDR sequence within the SEQ ID NO.

In one embodiment, the invention provides an antibody that binds to human CD103 comprising a heavy chain of the antibody that comprises the amino acid sequence of SEQ ID NO: 7 and a light chain of the antibody comprises the amino acid sequence of SEQ ID NO: 8.

In one embodiment, the invention provides an antibody that binds to human CD103 comprising a heavy chain of the antibody that comprises the amino acid sequence of SEQ ID NO: 15 and a light chain of the antibody that comprises the amino acid sequence of SEQ ID NO: 16.

In one embodiment, the invention provides an antibody that binds to human CD103 comprising a heavy chain of the antibody that comprises the amino acid sequence of SEQ ID NO: 23 and a light chain of the antibody that comprises the amino acid sequence of SEQ ID NO: 24.

In one embodiment, the invention provides an antibody that binds to human CD103 comprising a heavy chain of the antibody that comprises the amino acid sequence of SEQ ID NO: 31 and a light chain of the antibody that comprises the amino acid sequence of SEQ ID NO: 32.

In one embodiment, the invention provides an antibody that binds to human CD103 comprising a heavy chain of the antibody that comprises the amino acid sequence of SEQ ID NO: 39 and a light chain of the antibody that comprises the amino acid sequence of SEQ ID NO: 40.

In one embodiment, the invention provides an antibody that binds to human CD103 comprising a heavy chain of the antibody that comprises the amino acid sequence having at least 95% (and more preferably 97% or most preferably 99%) sequence identity to SEQ ID NO: 7, with any difference in such sequence being confined to amino acids that are not a part of a CDR sequence within SEQ ID NO: 7, and a light chain of the antibody comprises the amino acid sequence having at least 95% (and more preferably 97% or most preferably 99%) sequence identity to SEQ ID NO: 8, with any difference in such sequence being confined to amino acids that are not a part of a CDR sequence within SEQ ID NO: 8.

In one embodiment, the invention provides an antibody that binds to human CD103 comprising a heavy chain of the antibody that comprises the amino acid sequence having at least 95% (and more preferably 97% or most preferably 99%) sequence identity to SEQ ID NO: 15, with any difference in such sequence being confined to amino acids that are not a part of a CDR sequence within SEQ ID NO: 15, and a light chain of the antibody comprises the amino acid sequence having at least 95% (and more preferably 97% or most preferably 99%) sequence identity to SEQ ID NO: 16, with any difference in such sequence being confined to amino acids that are not a part of a CDR sequence within SEQ ID NO: 16.

In one embodiment, the invention provides an antibody that binds to human CD103 comprising a heavy chain of the antibody that comprises the amino acid sequence having at least 95% (and more preferably 97% or most preferably 99%) sequence identity to SEQ ID NO: 23, with any difference in such sequence being confined to amino acids that are not a part of a CDR sequence within SEQ ID NO: 23, and a light chain of the antibody comprises the amino acid sequence having at least 95% (and more preferably 97% or most preferably 99%) sequence identity to SEQ ID NO: 24, with any difference in such sequence being confined to amino acids that are not a part of a CDR sequence within SEQ ID NO: 24.

In one embodiment, the invention provides an antibody that binds to human CD103 comprising a heavy chain of the antibody that comprises the amino acid sequence having at least 95% (and more preferably 97% or most preferably 99%) sequence identity to SEQ ID NO: 31, with any difference in such sequence being confined to amino acids that are not a part of a CDR sequence within SEQ ID NO: 31, and a light chain of the antibody comprises the amino acid sequence having at least 95% (and more preferably 97% or most preferably 99%) sequence identity to SEQ ID NO: 32, with any difference in such sequence being confined to amino acids that are not a part of a CDR sequence within SEQ ID NO: 32.

In one embodiment, the invention provides an antibody that binds to human CD103 comprising a heavy chain of the antibody that comprises the amino acid sequence having at least 95% (and more preferably 97% or most preferably 99%) sequence identity to SEQ ID NO: 39, with any difference in such sequence being confined to amino acids that are not a part of a CDR sequence within SEQ ID NO: 39, and a light chain of the antibody comprises the amino acid sequence having at least 95% (and more preferably 97% or most preferably 99%) sequence identity to SEQ ID NO: 40, with any difference in such sequence being confined to amino acids that are not a part of a CDR sequence within SEQ ID NO: 40.

In this context, “sequence similarity” is based on the extent of identity combined with the extent of conservative changes. The percentage of “sequence similarity” is the percentage of amino acids or nucleotides which is either identical or conservatively changed viz. “sequence similarity”=percent sequence identity)+percent conservative changes). Thus, for the purpose of this invention “conservative changes” and “identity” are considered to be species of the broader term “similarity”. Thus, whenever the term sequence “similarity” is used it embraces sequence “identity” and “conservative changes”. According to certain embodiments the conservative changes are disregarded and the percent sequence similarity refers to percent sequence identity. In certain embodiments, the changes in a sequence permitted by the referenced percent sequence identity are all or nearly all conservative changes; that is, when a sequence is 90% identical, the remaining 10% are all or nearly all conservative changes. The term “nearly all” in this context refers to at least 75% of the permitted sequence changes are conservative changes, more preferably at least 85%, still more preferably at least 90%, and most preferably at least 95%. In certain embodiments of antibody heavy and/or light chains, the permitted sequence changes are within the framework regions and not in the CDRs.

In any of the above embodiments, the antibody or antigen binding fragment thereof may be isolated, as that term is defined herein.

In any of the above embodiments, the antibody or antigen binding fragment thereof is a recombinant antibody, as that term is defined herein.

In any of the above embodiments, the antibody or antigen binding fragment thereof is a full-length antibody, as that term is defined herein.

Antibodies or antigen binding fragments of the present invention may be obtained from a variety of species. For example, the antibodies of the present invention may comprise immunoglobulin sequences which are rabbit, mouse, rat, guinea pig, chicken, goat, sheep, donkey, human, llama or camelid sequences, or combinations of such sequences (so-called chimeric antibodies). Most preferably, the antibodies or antigen binding fragments are human antibodies or antigen binding fragments. Most preferably, the antibodies or antigen binding fragments are humanized antibodies or antigen binding fragments.

The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the V_{L}, V_{H}, C_{L} and C_{H}1 domains; (ii) a F(ab′)2 fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and VH domains of a single arm of an antibody, (v) a dAb fragment (Ward et al., (1989) Nature 341:544-546), which consists of a V_{H} domain; and (vi) an isolated complementarity determining region (CDR). Single chain antibodies are also included by reference in the term “antibody.” Preferred therapeutic antibodies are intact IgG antibodies. The term “intact IgG” as used herein is meant as a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, and IgG3. The known Ig domains in the IgG class of antibodies are V_{H}, Cγ1, Cγ2, Cγ3, V_{L}, and C_{L}.

In any of the above embodiments, the antibody or antigen binding fragment thereof is a human or humanized antibody comprising two heavy chains and two light chains. In one embodiment, the antibody is an IgG. In preferred embodiments, antibody is an IgG1, IgG2, or IgG4, and preferably a human IgG1, IgG2, or IgG4.

In one embodiment, the anti-CD103 antibody of the invention comprises a full length antibody structure having two light chains and two heavy chains as recited above, wherein each light chain comprises a human kappa light chain or a human lambda light chain constant domain; and each heavy chain comprises a human IgG1 constant region.

In one embodiment, the anti-CD103 antibody of the invention comprises a full length antibody structure having two light chains and two heavy chains as recited above, wherein each light chain comprises a human kappa light chain or a human lambda light chain constant domain; and each heavy chain comprises a human IgG2 constant region.

In one embodiment, the anti-CD103 antibody of the invention comprises a full-length antibody structure having two light chains and two heavy chains as recited above, wherein each light chain comprises a human kappa light chain or a human lambda light chain constant domain; and each heavy chain comprises a human IgG4 constant region.

In certain embodiments, the anti-CD103 antibody of the invention can be conjugated to at least one diagnostic label for in vivo imaging studies. In certain embodiments, the antigen binding fragment of anti-CD103 antibody of the invention can be conjugated to at least one diagnostic label for in vivo imaging studies. In certain embodiments, the anti-CD103 antibody of the invention can be conjugated to at least one therapeutic agent. In certain embodiments, the antigen binding fragment of anti-CD103 antibody of the invention can be conjugated to at least one therapeutic agent. In one embodiment, the therapeutic agent is a second antibody or fragment thereof. In one embodiment, the therapeutic agent is a second antibody. In one embodiment, the therapeutic agent is an immunomodulator. In one embodiment, the therapeutic agent is a hormone. In one embodiment, the therapeutic agent is a cytotoxic agent. In one embodiment, the therapeutic agent is an enzyme. In one embodiment, the therapeutic agent is a radionuclide. In one embodiment, the therapeutic agent is a second antibody conjugated to at least one immunomodulator. In one embodiment, the therapeutic agent is a second antibody conjugated to at least one one enzyme. In one embodiment, the therapeutic agent is a second antibody conjugated to at least one radioactive label. In one embodiment, the therapeutic agent is a second antibody conjugated to at least one hormone. In one embodiment, the therapeutic agent is a second antibody conjugated to at least one antisense oligonucleotide. In one embodiment, the therapeutic agent is a second antibody conjugated to at least one cytotoxic agent. In one embodiment, the therapeutic agent is a second antibody conjugated to a combination thereof. In one embodiment, the therapeutic agent is a combination of any one of a second antibody or fragment thereof, an immunomodulator, a hormone, a cytotoxic agent, an enzyme, a radionuclide, or a second antibody conjugated to at least one immunomodulator, enzyme, radioactive label, hormone, antisense oligonucleotide, or cytotoxic agent. In another embodiment the diagnostic label is one applicable to PET imaging. In another embodiment the diagnostic label is one applicable to single-photon emission computed tomography (SPECT) imaging. In another embodiment the diagnostic label is one applicable to MRI. In another embodiment the diagnostic label is one applicable to optical imaging. In another embodiment the diagnostic label is one applicable to (photo) acoustic imaging etc., such as ¹¹C, ¹³N, ¹⁵O, ^{99m}Tc, ⁶¹Cu, ⁶²Cu, ⁶⁴Cu, ⁶⁷Cu, ¹⁸F, ¹⁹F, ⁶⁶Ga, ⁶⁷Ga, ⁶⁸Ga, ⁷²Ga, ¹²³I, ¹²⁴I, ¹¹¹In, ¹⁷⁷Lu, ⁴⁴Sc, ⁴⁷Sc, ⁸⁶Y, ⁸⁸Y, ⁹⁰Y, ⁴⁵Ti, ⁸⁹Zr, indocyanine green, IRDye 800CW, fluorescein (FITC), magnetic (e.g., iron oxide) nanoparticles. This list is not meant to be limiting.

The invention also provides isolated nucleic acids encoding anyone of the anti-CD103 antibodies or antigen binding fragments of the invention.

The invention also provides expression vectors comprising one or more nucleic acids of the present invention. An expression vector is a DNA molecule comprising the regulatory elements necessary for transcription of a target nucleic acid in a host cell. Typically, the target nucleic acid is placed under the control of certain regulatory elements including constitutive or inducible promoters, tissue-specific regulatory elements, and enhancer elements. Such a target nucleic acid is said to be “operably linked to” the regulatory elements when the regulating element controls the expression of the gene.

These isolated nucleic acids and the expression vectors comprising them may be used to express the antibodies of the invention or antigen binding fragments thereof in recombinant host cells. Thus, the invention also provides host cells comprising an expression vector of the present invention.

The invention also provides a vessel or injection device comprising anyone of the anti-CD103 antibodies or antigen binding fragments of the invention.

The invention also provides a method of producing an anti-CD103 antibody or antigen binding fragment of the invention comprising: culturing a host cell comprising a polynucleotide encoding a heavy chain and/or light chain of an antibody of the invention (or an antigen binding fragment thereof) under conditions favorable to expression of the polynucleotide; and optionally, recovering the antibody or antigen binding fragment from the host cell and/or culture medium. In one embodiment, the polynucleotide encoding the heavy chain and the polynucleotide encoding the light chain are in a single vector. In another embodiment, the polynucleotide encoding the heavy chain and the polynucleotide encoding the light chain are in different vectors.

In another aspect, the invention relates to methods of imaging tissue or other biological specimen. These methods comprise contacting the biological specimen with an anti-CD103 antibody, and detecting the presence or amount of binding of the antibody to CD103 present in the biological specimen. Thus, an anti-CD103 antibody is used as an imaging agent.

In a related aspect, the invention relates to methods for producing an imaging agent comprising an anti-CD103 antibody and a diagnostic label. These methods comprise forming a covalent association between the anti-CD103 antibody and the diagnostic label. Alternatively, these methods comprise forming a non-covalent association between the anti-CD103 antibody and the diagnostic label. In the case of radiolabels, the isotope may be chelated using a bifunctional chelator comprising a first functional group for the immobilization of the radiometal and a second functional group for the covalent attachment to the antibody. Examples of such chelators include, but are not limited to, DOTA, NOTA, TRITA, TETA, TACN, cyclen, cyclam, homocyclen, EDTA, DTPA, DOTP, and NOTMP. Functionalized versions of the chelators provide a linkage chemistry which provides a terminal functional moiety which may be selected from the group consisting of protected or unprotected sulfhydryl moieties, protected or unprotected amine moieties, protected or unprotected hydroxyl moieties, primary amine-reactive moieties, sulfhydryl-reactive moieties, photoreactive moieties, carboxyl-reactive moieties, arginine-reactive moieties, and carbonyl-reactive moieties. Functionalized versions of the chelators provide a linkage chemistry which provides a terminal functional moiety (i.e. protected sulfhydryl moieties). Functionalized versions of the chelators provide a linkage chemistry which provides a terminal functional moiety (i.e. unprotected sulfhydryl moieties). Functionalized versions of the chelators provide a linkage chemistry which provides a terminal functional moiety (i.e. protected amine moieties). Functionalized versions of the chelators provide a linkage chemistry which provides a terminal functional moiety (i.e. unprotected amine moieties). Functionalized versions of the chelators provide a linkage chemistry which provides a terminal functional moiety (i.e. protected hydroxyl moieties). Functionalized versions of the chelators provide a linkage chemistry which provides a terminal functional moiety (i.e. unprotected hydroxyl moieties). Functionalized versions of the chelators provide a linkage chemistry which provides a terminal functional moiety (i.e. amine-reactive moieties). Functionalized versions of the chelators provide a linkage chemistry which provides a terminal functional moiety (i.e. sulfhydryl-reactive moieties). Functionalized versions of the chelators provide a linkage chemistry which provides a terminal functional moiety (i.e. photoreactive moieties). Functionalized versions of the chelators provide a linkage chemistry which provides a terminal functional moiety (i.e. carboxyl-reactive moieties). Functionalized versions of the chelators provide a linkage chemistry which provides a terminal functional moiety (i.e. arginine-reactive moieties). Functionalized versions of the chelators provide a linkage chemistry which provides a terminal functional moiety (i.e. carbonyl-reactive moieties). A direct labeling approach in which a reducing agent converts disulfide linkages to free thiols which bind to the radiolabel is also contemplated.

In various embodiments, the diagnostic label may be selected from the group consisting of an enzyme, a nucleic acid, a fluorophore, biotin, avidin, streptavidin, digoxigenin, maltose, oligohistidine, 2,4-dintrobenzene, phenylarsenate, a metal, a peptide tag, a fluorescent or colored microsphere, a fluorescent particle, and a colored latex particle. This list is not meant to be limiting. In various embodiments, the diagnostic label is an enzyme. In various embodiments, the diagnostic label is a nucleic acid. In various embodiments, the diagnostic label is a fluorophore. In various embodiments, the diagnostic label is biotin. In various embodiments, the diagnostic label is avidin. In various embodiments, the diagnostic label is streptavidin. In various embodiments, the diagnostic label is digoxigenin. In various embodiments, the diagnostic label is maltose. In various embodiments, the diagnostic label is oligohistidine. In various embodiments, the diagnostic label is 2,4-dintrobenzene. In various embodiments, the diagnostic label is phenylarsenate. In various embodiments, the diagnostic label is a metal. In various embodiments, the diagnostic label is a peptide tag. In various embodiments, the diagnostic label is a fluorescent microsphere. In various embodiments, the diagnostic label is a colored microsphere. In various embodiments, the diagnostic label is a fluorescent particle. In various embodiments, the diagnostic label is a colored latex particle. Such a label may be conjugated to the antibody by means of a crosslinker which contains a maleimide, an alkyl halide, an aryl halide, an alpha-haloacyl, an activated aryl, a pyridyl disulfide, a carbonyl, a carboxyl, a thiol, a thioester, disulfide, a N-hydroxy-succinimide, or a cyclic thiolactone, etc.

In certain embodiments, the biological specimen is tissue within a living body, and the method is an in vivo imaging method. In certain embodiments, the biological specimen is tissue within a living body, and the method is an in vivo imaging method such as PET imaging. In certain embodiments, the biological specimen is tissue within a living body, and the method is an in vivo imaging method single-photon emission computed tomography (SPECT) imaging. In certain embodiments, the biological specimen is tissue within a living body, and the method is an in vivo imaging method MRI. In these methods, the anti-CD103 antibody is detectably labeled according to the requirements of the imaging methodology employed. Suitable diagnostic labels are described herein, and include, but are not limited to, ¹¹C, ¹³N, ¹⁵O, ^{99m}Tc, ⁶¹Cu, ⁶²Cu, ⁶⁴Cu, ⁶⁷Cu, ¹⁸F, ¹⁹F, ⁶⁶Ga, ⁶⁷Ga, ⁶⁸Ga, ⁷²Ga, ¹²³I, ¹²⁴I, ¹¹¹In, ¹⁷⁷Lu, ⁴⁴Sc, ⁴⁷Sc, ⁸⁶Y, ⁸⁸Y, ⁹⁰Y, ⁴⁵Ti, ⁸⁹Zr, indocyanine green, IRDye 800CW, fluorescein (FITC), and magnetic (e.g., iron oxide) nanoparticles.

In various embodiments, the anti-CD103 antibody used as an imaging agent blocks CD103 binding to its cognate receptor E-cadherin. In various embodiments, the anti-CD103 antibody used as an imaging agent does not block CD103 binding to its cognate receptor E-cadherin. In various embodiments, the anti-CD103 antibody used as an imaging agent partially blocks CD103 binding to its cognate receptor E-cadherin. Examples of each of these types of anti-CD103 antibody are described hereinafter.

In various embodiments, the methods of imaging tissue or other biological specimen utilizes an antibody of the invention as an imaging agent.

In various embodiments, the methods of imaging tissue or other biological specimen utilizes an antigen binding fragment of an antibody of the invention as an imaging agent.

In various embodiments, the methods of imaging tissue or other biological specimen utilizes an antibody or antigen binding fragment thereof as an imaging agent which comprises:
a. a heavy chain variable region CDR1 comprising the amino acid sequence of SEQ ID NO: 1 or an amino acid sequence differing from SEQ ID NO: 1 by 1, 2, or 3 conservative substitutions,
b. a heavy chain variable region CDR2 comprising the amino acid sequence of SEQ ID NO: 2 or an amino acid sequence differing from SEQ ID NO: 2 by 1, 2, or 3 conservative substitutions,
c. a heavy chain variable region CDR3 comprising the amino acid sequence of SEQ ID NO: 3 or an amino acid sequence differing from SEQ ID NO: 3 by 1, 2, or 3 conservative substitutions,
d. a light chain variable region CDR1 comprising the amino acid sequence of SEQ ID NO: 4 or an amino acid sequence differing from SEQ ID NO: 4 by 1, 2, or 3 conservative substitutions,
e. a light chain variable region CDR2 comprising the amino acid sequence of SEQ ID NO: 5 or an amino acid sequence differing from SEQ ID NO: 5 by 1, 2, or 3 conservative substitutions, and
f. a light chain variable region CDR3 comprising the amino acid sequence of SEQ ID NO: 6 or an amino acid sequence differing from SEQ ID NO: 6 by 1, 2, or 3 conservative substitutions;
or
g. a heavy chain variable region CDR1 comprising the amino acid sequence of SEQ ID NO: 9 or an amino acid sequence differing from SEQ ID NO: 9 by 1, 2, or 3 conservative substitutions,
h. a heavy chain variable region CDR2 comprising the amino acid sequence of SEQ ID NO: 10 or an amino acid sequence differing from SEQ ID NO: 10 by 1, 2, or 3 conservative substitutions,
i. a heavy chain variable region CDR3 comprising the amino acid sequence of SEQ ID NO: 11 or an amino acid sequence differing from SEQ ID NO: 11 by 1, 2, or 3 conservative substitutions,
j. a light chain variable region CDR1 comprising the amino acid sequence of SEQ ID NO: 12 or an amino acid sequence differing from SEQ ID NO: 12 by 1, 2, or 3 conservative substitutions,
k. a light chain variable region CDR2 comprising the amino acid sequence of SEQ ID NO: 13 or an amino acid sequence differing from SEQ ID NO: 13 by 1, 2, or 3 conservative substitutions, and
l. a light chain variable region CDR3 comprising the amino acid sequence of SEQ ID NO: 14 or an amino acid sequence differing from SEQ ID NO: 14 by 1, 2, or 3 conservative substitutions;
or
m. a heavy chain variable region CDR1 comprising the amino acid sequence of SEQ ID NO: 17 or an amino acid sequence differing from SEQ ID NO: 17 by 1, 2, or 3 conservative substitutions,
n. a heavy chain variable region CDR2 comprising the amino acid sequence of SEQ ID NO: 18 or an amino acid sequence differing from SEQ ID NO: 18 by
o. a heavy chain variable region CDR3 comprising the amino acid sequence of SEQ ID NO: 19 or an amino acid sequence differing from SEQ ID NO: 19 by 1, 2, or 3 conservative substitutions,
p. a light chain variable region CDR1 comprising the amino acid sequence of SEQ ID NO: 20 or an amino acid sequence differing from SEQ ID NO: 20 by 1, 2, or 3 conservative substitutions,
q. a light chain variable region CDR2 comprising the amino acid sequence of SEQ ID NO: 21 or an amino acid sequence differing from SEQ ID NO: 21 by 1, 2, or 3 conservative substitutions, and
r. a light chain variable region CDR3 comprising the amino acid sequence of SEQ ID NO: 22 or an amino acid sequence differing from SEQ ID NO: 22 by 1, 2, or 3 conservative substitutions;
or
s. a heavy chain variable region CDR1 comprising the amino acid sequence of SEQ ID NO: 25 or an amino acid sequence differing from SEQ ID NO: 25 by 1, 2, or 3 conservative substitutions,
t. a heavy chain variable region CDR2 comprising the amino acid sequence of SEQ ID NO: 26 or an amino acid sequence differing from SEQ ID NO: 26 by 1, 2, or 3 conservative substitutions,
u. a heavy chain variable region CDR3 comprising the amino acid sequence of SEQ ID NO: 27 or an amino acid sequence differing from SEQ ID NO: 27 by 1, 2, or 3 conservative substitutions,
v. a light chain variable region CDR1 comprising the amino acid sequence of SEQ ID NO: 28 or an amino acid sequence differing from SEQ ID NO: 28 by
w. a light chain variable region CDR2 comprising the amino acid sequence of SEQ ID NO: 29 or an amino acid sequence differing from SEQ ID NO: 29 by 1, 2, or 3 conservative substitutions, and
x. a light chain variable region CDR3 comprising the amino acid sequence of SEQ ID NO: 30 or an amino acid sequence differing from SEQ ID NO: 30 by 1, 2, or 3 conservative substitutions;
or
y. a heavy chain variable region CDR1 comprising the amino acid sequence of SEQ ID NO: 33 or an amino acid sequence differing from SEQ ID NO: 33 by 1, 2, or 3 conservative substitutions,
z. a heavy chain variable region CDR2 comprising the amino acid sequence of SEQ ID NO: 34 or an amino acid sequence differing from SEQ ID NO: 34 by 1, 2, or 3 conservative substitutions,
aa. a heavy chain variable region CDR3 comprising the amino acid sequence of SEQ ID NO: 35 or an amino acid sequence differing from SEQ ID NO: 35 by 1, 2, or 3 conservative substitutions,
bb. a light chain variable region CDR1 comprising the amino acid sequence of SEQ ID NO: 36 or an amino acid sequence differing from SEQ ID NO: 36 by 1, 2, or 3 conservative substitutions,
cc. a light chain variable region CDR2 comprising the amino acid sequence of SEQ ID NO: 37 or an amino acid sequence differing from SEQ ID NO: 37 by 1, 2, or 3 conservative substitutions, and
dd. a light chain variable region CDR3 comprising the amino acid sequence of SEQ ID NO: 38 or an amino acid sequence differing from SEQ ID NO: 38 by 1, 2, or 3 conservative substitutions.

In various other embodiments, the methods of imaging tissue or other biological specimen utilizes an antibody or antigen binding fragment thereof as an imaging agent which comprises:
a. a heavy chain of the antibody or antigen binding fragment that comprises the amino acid sequence of SEQ ID NO: 7 and a light chain of the antibody or antigen binding fragment that comprises the amino acid sequence of SEQ ID NO: 8; or
b. a heavy chain of the antibody or antigen binding fragment that comprises the amino acid sequence of SEQ ID NO: 15 and a light chain of the antibody or antigen binding fragment that comprises the amino acid sequence of SEQ ID NO: 16; or
c. a heavy chain of the antibody or antigen binding fragment that comprises the amino acid sequence of SEQ ID NO: 23 and a light chain of the antibody or antigen binding fragment that comprises the amino acid sequence of SEQ ID NO: 24; or
d. a heavy chain of the antibody or antigen binding fragment that comprises the amino acid sequence of SEQ ID NO: 31 and a light chain of the antibody or antigen binding fragment that comprises the amino acid sequence of SEQ ID NO: 32; or
e. a heavy chain of the antibody or antigen binding fragment that comprises the amino acid sequence of SEQ ID NO: 39 and a light chain of the antibody or antigen binding fragment that comprises the amino acid sequence of SEQ ID NO: 40;
or in each case a sequence having at least 95% (and more preferably 97% or 99%) sequence similarity or identity to a given SEQ ID NO, with any difference in such sequence being confined to amino acids that are not a part of a CDR sequence within the SEQ ID NO.

In another aspect, the invention relates to methods for the use of an anti-CD103 antibody of the invention as a therapeutic agent. In a related aspect, the invention relates to the use of an anti-CD103 antibody of the invention in the manufacture of a medicament. In another related aspect, the invention relates to methods for the use of an anti-CD103 antibody of the invention to inhibit CD103 signaling. In another related aspect, the invention relates to methods for the use of an anti-CD103 antibody of the invention to block CD103 binding to E-cadherin.

In one embodiment, the methods are for treating a CD103 signaling-mediated condition in an individual in need thereof, and the methods comprise administering an effective amount of an anti-CD103 antibody of the invention to the individual wherein the anti-CD103 antibody is optionally coupled to a cytotoxic agent.

In one embodiment, the methods are for treating a CD103 signaling-mediated condition in an individual in need thereof, and the methods comprise administering an effective amount of an antigen binding fragment of an anti-CD103 antibody of the invention to the individual wherein the anti-CD103 antibody is optionally coupled to a cytotoxic agent.

In one embodiment, the methods are for preventing a CD103 signaling-mediated condition in an individual in need thereof, and the methods comprise administering an effective amount of an anti-CD103 antibody of the invention, or antigen binding fragment thereof, to the individual wherein the anti-CD103 antibody is optionally coupled to a cytotoxic agent

In one embodiment, the methods are for preventing a CD103 signaling-mediated condition in an individual in need thereof, and the methods comprise administering an effective amount of an antigen binding fragment of an anti-CD103 antibody of the invention to the individual wherein the anti-CD103 antibody is optionally coupled to a cytotoxic agent.

In another embodiment, the methods are for inhibiting CD103 signaling in a cell, and the methods comprise contacting the cell with an anti-CD103 antibody of the invention, or antigen binding fragment thereof.

In another embodiment, the methods are for inhibiting CD103 binding to E-cadherin present on a cell, and the methods comprise contacting the cell with an anti-CD103 antibody of the invention, or antigen binding fragment thereof.

In yet another embodiment, the methods are for depleting CD103-expressing cells in an individual, and the methods comprise administering an effective amount of an anti-CD103 antibody of the invention, or antigen binding fragment thereof, to the individual wherein the anti-CD103 antibody is optionally coupled to a cytotoxic agent.

In yet another embodiment, the methods are for method for treating or preventing a disease selected from the group consisting of Hairy Cell leukemia, HCLv, intestinal and extraintestinal lymphomas, enteropathy-associated T-cell lymphoma (EATL), T-lymphoblastic leukemia/lymphoma (T-ALL), T-cell prolymphocytic leukemia (T-PLL), adult T cell leukemia/lymphoma (ATLL), mycosis fungoides (MF), anaplastic large cell lymphoma ALCL, cutaneous T-cell lymphoma (CTCL), Sezary Syndrome (SS), Alzheimer's disease, Parkinson's disease, multiple sclerosis, IgM polyneuropathies, myasthenia gravis, atopic dermatitis, allergy, asthma, systemic inflammatory response syndrome (SIRS), sepsis, septic shock, atherosclerosis, celiac disease, dermatomyositis, scleroderma, interstitial cystitis, transplant rejection, graft-versus-host disease, Aicardi-Goutieres Syndrome, Hutchison Guilford progeria syndrome, Singleton-Merten Syndrome, proteasome-associated autoinflammatory syndrome, SAVI (STING-associated vasculopathy with onset in infancy), CANDLE (Chronic Atypical Neutrophilic Dermatosis with Lipodystrophy and Elevated Temperature) syndrome, chilblain lupus erythematosus, systemic lupus erythematosus, rheumatoid arthritis, juvenile rheumatoid arthritis, Wegener's disease, inflammatory bowel disease (e.g. ulcerative colitis, Crohn's disease), idiopathic thrombocytopenia purpura, thrombotic thrombocytopeniaurpura, autoimmune thrombocytopenia, multiple sclerosis, psoriasis, IgA nephropathy, IgM polyneuropathies, glomerulonephritis, autoimmune myocarditis, myasthenia gravis, vasculitis, Type 1 diabetes, Type 2 diabetes, Sjorgen's syndrome, X-linked reticulate pigmentary disorder, polymyositis, spondyloenchondrodysplasia, and age-related macular degeneration in an individual in need thereof, and the methods comprise administering an effective amount of an anti-CD103 antibody of the invention, or antigen binding fragment thereof, to the individual wherein the anti-CD103 antibody is optionally coupled to a cytotoxic agent.

In yet another embodiment, the methods are for method for treating a disease selected from the group consisting of Hairy Cell leukemia, HCLv, intestinal and extraintestinal lymphomas, enteropathy-associated T-cell lymphoma (EATL), T-lymphoblastic leukemia/lymphoma (T-ALL), T-cell prolymphocytic leukemia (T-PLL), adult T cell leukemia/lymphoma (ATLL), mycosis fungoides (MF), anaplastic large cell lymphoma ALCL, cutaneous T-cell lymphoma (CTCL), Sezary Syndrome (SS), Alzheimer's disease, Parkinson's disease, multiple sclerosis, IgM polyneuropathies, myasthenia gravis, atopic dermatitis, allergy, asthma, systemic inflammatory response syndrome (SIRS), sepsis, septic shock, atherosclerosis, celiac disease, dermatomyositis, scleroderma, interstitial cystitis, transplant rejection, graft-versus-host disease, Aicardi-Goutieres Syndrome, Hutchison Guilford progeria syndrome, Singleton-Merten Syndrome, proteasome-associated autoinflammatory syndrome, SAVI (STING-associated vasculopathy with onset in infancy), CANDLE (Chronic Atypical Neutrophilic Dermatosis with Lipodystrophy and Elevated Temperature) syndrome, chilblain lupus erythematosus, systemic lupus erythematosus, rheumatoid arthritis, juvenile rheumatoid arthritis, Wegener's disease, inflammatory bowel disease (e.g. ulcerative colitis, Crohn's disease), idiopathic thrombocytopenia purpura, thrombotic thrombocytopeniaurpura, autoimmune thrombocytopenia, multiple sclerosis, psoriasis, IgA nephropathy, IgM polyneuropathies, glomerulonephritis, autoimmune myocarditis, myasthenia gravis, vasculitis, Type 1 diabetes, Type 2 diabetes, Sjorgen's syndrome, X-linked reticulate pigmentary disorder, polymyositis, spondyloenchondrodysplasia, and age-related macular degeneration in an individual in need thereof, and the methods comprise administering an effective amount of an anti-CD103 antibody of the invention to the individual wherein the anti-CD103 antibody is optionally coupled to a cytotoxic agent.

In yet another embodiment, the methods are for method for preventing a disease selected from the group consisting of Hairy Cell leukemia, HCLv, intestinal and extraintestinal lymphomas, enteropathy-associated T-cell lymphoma (EATL), T-lymphoblastic leukemia/lymphoma (T-ALL), T-cell prolymphocytic leukemia (T-PLL), adult T cell leukemia/lymphoma (ATLL), mycosis fungoides (MF), anaplastic large cell lymphoma ALCL, cutaneous T-cell lymphoma (CTCL), Sezary Syndrome (SS), Alzheimer's disease, Parkinson's disease, multiple sclerosis, IgM polyneuropathies, myasthenia gravis, atopic dermatitis, allergy, asthma, systemic inflammatory response syndrome (SIRS), sepsis, septic shock, atherosclerosis, celiac disease, dermatomyositis, scleroderma, interstitial cystitis, transplant rejection, graft-versus-host disease, Aicardi-Goutieres Syndrome, Hutchison Guilford progeria syndrome, Singleton-Merten Syndrome, proteasome-associated autoinflammatory syndrome, SAVI (STING-associated vasculopathy with onset in infancy), CANDLE (Chronic Atypical Neutrophilic Dermatosis with Lipodystrophy and Elevated Temperature) syndrome, chilblain lupus erythematosus, systemic lupus erythematosus, rheumatoid arthritis, juvenile rheumatoid arthritis, Wegener's disease, inflammatory bowel disease (e.g. ulcerative colitis, Crohn's disease), idiopathic thrombocytopenia purpura, thrombotic thrombocytopenia purpura, autoimmune thrombocytopenia, multiple sclerosis, psoriasis, IgA nephropathy, IgM polyneuropathies, glomerulonephritis, autoimmune myocarditis, myasthenia gravis, vasculitis, Type 1 diabetes, Type 2 diabetes, Sjorgen's syndrome, X-linked reticulate pigmentary disorder, polymyositis, spondyloenchondrodysplasia, and age-related macular degeneration in an individual in need thereof, and the methods comprise administering an effective amount of an anti-CD103 antibody of the invention to the individual wherein the anti-CD103 antibody is optionally coupled to a cytotoxic agent.

In various embodiments, these methods utilize an antibody of the invention.

In various embodiments, these methods utilize antigen binding fragment of an antibody of the invention.

In various embodiments, these methods utilize an antibody or antigen binding fragment thereof which comprises:
a. a heavy chain variable region CDR1 comprising the amino acid sequence of SEQ ID NO: 1 or an amino acid sequence differing from SEQ ID NO: 1 by 1, 2, or 3 conservative substitutions,
b. a heavy chain variable region CDR2 comprising the amino acid sequence of SEQ ID NO: 2 or an amino acid sequence differing from SEQ ID NO: 2 by 1, 2, or 3 conservative substitutions,
c. a heavy chain variable region CDR3 comprising the amino acid sequence of SEQ ID NO: 3 or an amino acid sequence differing from SEQ ID NO: 3 by 1, 2, or 3 conservative substitutions,
d. a light chain variable region CDR1 comprising the amino acid sequence of SEQ ID NO: 4 or an amino acid sequence differing from SEQ ID NO: 4 by 1, 2, or 3 conservative substitutions,
e. a light chain variable region CDR2 comprising the amino acid sequence of SEQ ID NO: 5 or an amino acid sequence differing from SEQ ID NO: 5 by 1, 2, or 3 conservative substitutions, and
f. a light chain variable region CDR3 comprising the amino acid sequence of SEQ ID NO: 6 or an amino acid sequence differing from SEQ ID NO: 6 by 1, 2, or 3 conservative substitutions;
or
g. a heavy chain variable region CDR1 comprising the amino acid sequence of SEQ ID NO: 9 or an amino acid sequence differing from SEQ ID NO: 9 by 1, 2, or 3 conservative substitutions,
h. a heavy chain variable region CDR2 comprising the amino acid sequence of SEQ ID NO: 10 or an amino acid sequence differing from SEQ ID NO: 10 by 1, 2, or 3 conservative substitutions,
i. a heavy chain variable region CDR3 comprising the amino acid sequence of SEQ ID NO: 11 or an amino acid sequence differing from SEQ ID NO: 11 by 1, 2, or 3 conservative substitutions,
j. a light chain variable region CDR1 comprising the amino acid sequence of SEQ ID NO: 12 or an amino acid sequence differing from SEQ ID NO: 12 by
k. a light chain variable region CDR2 comprising the amino acid sequence of SEQ ID NO: 13 or an amino acid sequence differing from SEQ ID NO: 13 by 1, 2, or 3 conservative substitutions, and
l. a light chain variable region CDR3 comprising the amino acid sequence of SEQ ID NO: 14 or an amino acid sequence differing from SEQ ID NO: 14 by 1, 2, or 3 conservative substitutions;
or
m. a heavy chain variable region CDR1 comprising the amino acid sequence of SEQ ID NO: 17 or an amino acid sequence differing from SEQ ID NO: 17 by 1, 2, or 3 conservative substitutions,
n. a heavy chain variable region CDR2 comprising the amino acid sequence of SEQ ID NO: 18 or an amino acid sequence differing from SEQ ID NO: 18 by 1, 2, or 3 conservative substitutions,
o. a heavy chain variable region CDR3 comprising the amino acid sequence of SEQ ID NO: 19 or an amino acid sequence differing from SEQ ID NO: 19 by 1, 2, or 3 conservative substitutions,
p. a light chain variable region CDR1 comprising the amino acid sequence of SEQ ID NO: 20 or an amino acid sequence differing from SEQ ID NO: 20 by 1, 2, or 3 conservative substitutions,
q. a light chain variable region CDR2 comprising the amino acid sequence of SEQ ID NO: 21 or an amino acid sequence differing from SEQ ID NO: 21 by 1, 2, or 3 conservative substitutions, and
r. a light chain variable region CDR3 comprising the amino acid sequence of SEQ ID NO: 22 or an amino acid sequence differing from SEQ ID NO: 22 by 1, 2, or 3 conservative substitutions;
or
s. a heavy chain variable region CDR1 comprising the amino acid sequence of SEQ ID NO: 25 or an amino acid sequence differing from SEQ ID NO: 25 by 1, 2, or 3 conservative substitutions,
t. a heavy chain variable region CDR2 comprising the amino acid sequence of SEQ ID NO: 26 or an amino acid sequence differing from SEQ ID NO: 26 by 1, 2, or 3 conservative substitutions,
u. a heavy chain variable region CDR3 comprising the amino acid sequence of SEQ ID NO: 27 or an amino acid sequence differing from SEQ ID NO: 27 by 1, 2, or 3 conservative substitutions,
v. a light chain variable region CDR1 comprising the amino acid sequence of SEQ ID NO: 28 or an amino acid sequence differing from SEQ ID NO: 28 by 1, 2, or 3 conservative substitutions,
w. a light chain variable region CDR2 comprising the amino acid sequence of SEQ ID NO: 29 or an amino acid sequence differing from SEQ ID NO: 29 by 1, 2, or 3 conservative substitutions, and
x. a light chain variable region CDR3 comprising the amino acid sequence of SEQ ID NO: 30 or an amino acid sequence differing from SEQ ID NO: 30 by 1, 2, or 3 conservative substitutions;
or
y. a heavy chain variable region CDR1 comprising the amino acid sequence of SEQ ID NO: 33 or an amino acid sequence differing from SEQ ID NO: 33 by 1, 2, or 3 conservative substitutions,
z. a heavy chain variable region CDR2 comprising the amino acid sequence of SEQ ID NO: 34 or an amino acid sequence differing from SEQ ID NO: 34 by
aa. a heavy chain variable region CDR3 comprising the amino acid sequence of SEQ ID NO: 35 or an amino acid sequence differing from SEQ ID NO: 35 by 1, 2, or 3 conservative substitutions,
bb. a light chain variable region CDR1 comprising the amino acid sequence of SEQ ID NO: 36 or an amino acid sequence differing from SEQ ID NO: 36 by 1, 2, or 3 conservative substitutions,
cc. a light chain variable region CDR2 comprising the amino acid sequence of SEQ ID NO: 37 or an amino acid sequence differing from SEQ ID NO: 37 by 1, 2, or 3 conservative substitutions, and
dd. a light chain variable region CDR3 comprising the amino acid sequence of SEQ ID NO: 38 or an amino acid sequence differing from SEQ ID NO: 38 by 1, 2, or 3 conservative substitutions.

It is to be understood that the invention is capable of embodiments in addition to those described and of being practiced and carried out in various ways. Also, it is to be understood that the phraseology and terminology employed herein, as well as the abstract, are for the purpose of description and should not be regarded as limiting. As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE FIGURES

FIG. 1: cell ELISA binding data of anti-hCD103 mAbs to CHO.K1-hCD103/hBeta7, CHO.K1-rhCD103/rhBeta7, and CHO.K1-hAlpha4/hBeta7.

FIG. 2: Phylogenetic tree for the different hCD103 VL and VH sequences.

FIG. 3: CELISA binding data of hCD103.01A, hCD103.05A, hCD103.06A, Fab.hCD103.01.C1, Fab.hCD103.05.C1, and Fab.hCD103.06.C1 to CHO.K1.hCD103/hBeta7, CHO.K1, and recombinant human CD103/Beta7.

FIG. 4: Cellular binding of AF647-hCD103.01A, AF647-hCD103.05A, AF647-hCD103.06A, AF647-Fab.hCD103.01.C1, AF647-Fab.hCD103.05.C1, and AF647-Fab.hCD103.06.C1 to CHO.K1.hCD103/hBeta7, respectively.

FIG. 5: Representative staining of tumor digest for CD8 and CD103 with control antibodies or anti-hCD103 mAbs.

FIG. 6: Binding of anti-hCD103 mAb candidates to CD3+ cells (total T-cell population), CD3+ CD103+ CD8+ cells (T-cell subpopulation), and CD33+ cells (myeloid population). N=10 different tumor digests.

FIG. 7: Staining of tumor digest for CD8 and CD103 with control reagents or anti-hCD103 Fabs. Top two rows show staining with parental Fabs (non-labeled) and detection with a secondary anti-Fab reagent, while bottom row shows results for staining with AF647 conjugated Fabs and direct read-out.

FIG. 8: CD103+ CD8+ T cells were pre-incubated with our CD103 mAbs or the commercial CD103 mAb (Clone BerACT-8, BD bioscience) and subsequently incubated with their fluorescently labeled counterparts to study differences in affinity and competition between the mAbs. Percentage binding of fluorescently labeled mAbs was determined using flow cytometry. Maximum binding was set at 100%.

FIG. 9: CD103+ T cell binding to recombinant E-cadherin in the presence of anti-hCD103 mAbs and controls. Antibodies were pre-incubated with cells before incubation with recombinant E-cadherin (left set of bars, pre-treatment) or cells were first incubated with recombinant E-cadherin whereafter the mAbs were added (right set of bars, treatment).

FIG. 10: Binding of Df-conjugated and parental anti-hCD103 mAbs (candidates hCD103.01A and hCD103.05A), or Fab fragments (candidates Fab.hCD103.01.C1 and Fab.hCD103.05.C1) to CHO.K1-hCD103/hBeta7.

FIG. 11: Radiochemical purity of ⁸⁹Zr-labeled anti-hCD103 mAbs (candidates hCD103.01A and hCD103.05A) at different ⁸⁹Zr levels.

FIG. 12: Binding of ⁸⁹Zr-labeled anti-hCD103 mAbs (candidates hCD103.01A and hCD103.05A) to CHO.K1-hCD103/hBeta7 (red) and CHO.K1 (blue). Binding was measured as amount of ⁸⁹Zr-mAb bound activity.

FIG. 13A-E: PET imaging protocol for ⁸⁹Zr-hCD103.01A and ⁸⁹Zr-hCD103.05A (13A), PET imaging 2D visualization (coronals) (13B), target to blood ratio of ⁸⁹Zr-hCD103.01A and ⁸⁹Zr-hCD103.05A, and ⁸⁹Zr-hCD103.01A and ⁸⁹Zr-hCD103.05A levels in blood versus target tissue, in CHO.K1-hCD103/hBeta7 or CHO.K1 WT bearing mice (13C and 13D), and comparative tissue distribution ratios (13E). Tumor (target) here means CHO.K1-hCD103/hBeta7 (red and green) or CHO.K1 WT (grey), respectively. CHO.K1 WT bearing mice were injected with ⁸⁹Zr-hCD103.01A as an unspecific control group.

FIG. 14: Biodistribution results for ⁸⁹Zr-hCD103.01A and ⁸⁹Zr-hCD103.05A in CHO.K1-hCD103/hBeta7 or CHO.K1 WT bearing mice (n=3) 6 days post-injection. Tumor here means CHO.K1-hCD103/hBeta7 (red or green) or CHO.K1 (grey), respectively. CHO.K1 WT bearing mice were injected with ⁸⁹Zr-hCD103.01A as an unspecific control group.

FIG. 15: Biodistribution results for ⁸⁹Zr-Fab.hCD103.01.C1 and ⁸⁹Zr-Fab.hCD103.05.C1 in CHO.K1-hCD103/hBeta7 (n=2) or CHO.K1 WT bearing mice (n=3) 24 hours post-injection. Tumor here means CHO.K1-hCD103/hBeta7 (red or green) or CHO.K1 WT (grey), respectively. CHO.K1 WT bearing mice were injected with ⁸⁹Zr-Fab.hCD103.01.C1 as an unspecific control group.

### DETAILED DESCRIPTION

Most, if not all forms of cancer immunotherapy rely on the induction of T cell-based immune responses against antigens preferentially or selectively expressed in cancer cells and presented via major histocompatibility molecules (MHC) on the cell surface. This mode of action is perhaps exemplified most by the exquisite responses of patients with a high tumor mutational burden (TMB) to treatment with monoclonal antibodies that block programmed death-1 (PD-1) or its ligand (PD-L1). In an effort to extend the promise of immunotherapy to more patients, over 2000 (combination) immunotherapy trials have now been initiated across numerous types of cancer. Considering this plethora of treatment options, biomarkers that can guide drug development, treatment decisions and assess therapeutic effects, are urgently needed.

A hallmark of successful immunotherapy is an increase in the activity and number of T cells within the tumor mass (tumor-infiltrating lymphocytes; TIL). TIL “load” in tumor lesions therefore represents an attractive biomarker to support selection and monitoring of patients for immunotherapy. Unfortunately, there is a wide repertoire of TIL and not every T cell within a tumor is involved in the anti-cancer immune response. In recent years, the integrin subunit CD103 has come forward as a marker of TIL for prognostic benefit across epithelial malignancies, including, esophageal, melanoma, lung, breast, bladder and all gynecological cancers. Importantly, CD103⁺ TIL comprise the CD39⁺, PD-1⁺ and CD137⁺ TIL populations previously linked to anti-cancer effects in tumors. Mechanistic studies have also demonstrated CD103 to be induced after specific activation of T cells against their cognate target, and CD103⁺ cells expand significantly during successful anti-PD-1 treatment in melanoma, esophageal squamous cell carcinoma and non-small cell lung cancer patients. Finally, CD103 is absent from other immune cell populations in the tumor and therefore provides excellent cell specificity. Taken together, intratumoral CD103 detection may provide an excellent biomarker for determining TIL load and responses to immunotherapy.

The current standard for assessing TIL load is through immunohistochemistry (IHC) on tissue biopsies. However, there are several obstacles known to be associated with biopsy-based techniques, such as poor accessibility of lesions, burden for the patient, tumor heterogeneity within and between lesions and sampling errors. Also using current technologies, it is difficult/not possible to follow T-cell infiltration in tumor lesions over time. In order to overcome these obstacles and obtain information about TIL load in all tumor lesions and toxicity-sensitive organs in the patient, noninvasive whole-body imaging techniques can be applied. Positron emission tomography (PET) is a molecular imaging technique that allows repetitive, non-invasive clinical assessment of tumor characteristics such as the expression of hormone and growth factor receptors. PET is characterized by a high spatial resolution, sensitivity, and possibility to quantify the imaging signal. PET could enable specific monitoring of CD103⁺ cells in the tumor, provided that a suitably sensitive radiopharmaceutical is available. Therefore, here we describe the development of various anti-CD103 specific antibodies that are suitable for radiopharmaceutical use.

In addition to a diagnostic/prognostic target, CD103 also presents a therapeutic target in a variety of diseases. For example, CD103 is expressed in several subsets of lymphocytes including T cells, intestinal intraepithelial lymphocytes and lamina propria lymphocytes. Interaction between CD103 and E-cadherin results in adhesion of lymphocytes to epithelial cells. While E-cadherin is constitutively expressed in epithelial cells, the expression of CD103 is induced in T cells upon inflammatory stimulation in vitro. Blockade of CD103 is of particular relevance in disorders involving expansion of CD8+ and Th9 cells such as inflammatory bowel disease and in allograft rejection. CD103 is also expressed by dendritic cells and on various T cell types, including malignant forms of these cells. Additionally, tumor-associated CD103+ CD8 T cells can have a tolerogenic phenotype, and CD103⁺ DCs show expression of immunomodulatory molecules and produce immunosuppressive factors such as IL-10, TGF-β, IL-35, and indoleamine 2,3-dioxygenase (IDO), resulting in T cell anergy and apoptosis and induction of Tregs.

Thus, molecules that bind to CD103 and interfere with its interaction between CD103 and E-cadherin are drug candidates for the diseases. Likewise, molecules that bind to CD103 may be used, for example as part of an antibody-drug conjugate, to deplete CD103-expressing cells for therapeutic purposes.

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell.

“Treat” or “treating” means to administer a therapeutic agent, such as a composition containing any of the antibodies or antigen-binding fragments of the present invention, internally or externally to a subject or patient having one or more disease symptoms, or being suspected of having a disease, for which the agent has therapeutic activity. Typically, the agent is administered in an amount effective to alleviate one or more disease symptoms in the treated subject or population, whether by inducing the regression of or inhibiting the progression of such symptom(s) by any clinically measurable degree. The amount of a therapeutic agent that is effective to alleviate any particular disease symptom may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom.

“Recombinant expression” of a protein means the transcription and translation of an exogenous gene in a host organism to generate the protein, which is referred to herein as a “recombinant protein.”

The term “Positron Emission Tomography (PET)” as used herein refers to a nuclear imaging technique used in the medical field to assist in the diagnosis of diseases. PET allows the physician to examine the whole patient at once by producing pictures of many functions of the human body unobtainable by other imaging techniques. In this regard, PET displays images of how the body works (physiology or function) instead of simply how it looks. Applications for PET imaging include those in the fields of oncology, cardiology, and neurology. In PET, short-lived positron-emitting isotopes, herein referred to as radiopharmaceuticals, are injected into a patient. When these radioactive drugs are administered to a patient, they distribute within the body according to the physiologic pathways associated with their stable counterparts.

The term “SPECT” as used herein refers to “Single-Photon Emission Computed Tomography which is a nuclear medicine tomographic imaging technique using gamma rays. It is very similar to conventional nuclear medicine planar imaging using a gamma camera and able to provide true 3D information. This information is typically presented as cross-sectional slices through the patient, but can be freely reformatted or manipulated as required. The basic technique requires delivery of a gamma-emitting radioisotope (called radionuclide) into the patient, normally through injection into the bloodstream.

By the term “detectable label” is meant, for the purposes of the specification or claims, a label molecule that is attached indirectly or directly to an antibody or antigen-binding fragment thereof according to the disclosure, wherein the label molecule facilitates the detection of the antibody in which it is incorporated. Thus, “detectable label” is used synonymously with “label molecule”.

The term “imaging agent” as used herein refers to a labeling moiety that is useful for providing an indication of the position of the label and adherents thereto, in a cell or tissue of an animal or human subject, or a cell or tissue under in vitro conditions. While agents may include those that provide detectable signals such as fluorescence, luminescence, radioactivity, or can be detected by such methods as MRI imaging, and the like, in the context of the probes and methods of use of the disclosure, the term “imaging agent” particularly refers to a label detectable by such as PET or SPECT imaging technology such as, but not limited to, ⁶⁴Cu, ⁶⁷Cu, ⁸⁹Zr, ¹²⁴I, 86Y, ⁹⁰Y, ¹¹¹In, ^{123/131}I, ¹⁷⁷Lu, ¹⁸F, ^{99m}Tc, and the like. In the most preferred embodiments of the immunoconjugate probes of the disclosure the labeling agent is 89-zirconium (Zr) although it is contemplated that any metal isotope (or any other PET-compatible labeling agent) may be used that provides a PET-generated image and may be attached or conjugated to the glypican-3 targeting antibody or antibody fragment.

The term “biological specimen” refers to a tissue, body fluid, or other sample from or in an organism (e.g., a human patient) or from components (e.g., cells) of an organism. The sample may be of any biological tissue or fluid. The specimen may be a “clinical sample” which is a sample derived from a patient. Such specimen include, but are not limited to, sputum, blood, blood cells (e.g., white cells), amniotic fluid, plasma, bone marrow, and tissue or fine needle biopsy samples, urine, peritoneal fluid, and pleural fluid, or cells therefrom. Biological specimen may also include tissues or sections of tissues (such as frozen or parafin-embedded sections) taken for histological purposes. A biological specimen may also be referred to as a “patient sample.”

In certain embodiments a biological specimen may be a tumor, either within a living being or removed therefrom. The term “tumor” as used herein refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth. In particular, the probes and compositions of the disclosure are most advantageous for the detection of a cancer cells of the liver (hepatocellular carcinoma) and especially of such cells bearing epitopes of the glypican-3 membrane-bound protein.

### Anti-CD103 Antibodies and Antigen-Binding Fragments Thereof

The present invention provides antibodies that bind human CD103 and uses of such antibodies. The present invention provides antigen-binding fragments that bind human CD103 and uses of such fragments. In some embodiments, the anti-CD103 antibodies are isolated.

Whether an antibody specifically binds to a polypeptide sequence (e.g., human CD103) can be determined using any assay known in the art. Examples of assays known in the art to determining binding affinity include surface plasmon resonance (e.g., BIACORE) or a similar technique (e.g. KinExa or OCTET).

As used herein, the term “antibody” refers to any form of antibody that exhibits the desired biological activity. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the V_{L}, V_{H}, C_{L} and C_{H}1 domains; (ii) a F(ab′)2 fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Single chain antibodies are also included by reference in the term “antibody.” Preferred therapeutic antibodies are intact IgG antibodies. The term “intact IgG” as used herein is meant as a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3. The known Ig domains in the IgG class of antibodies are V_{H}, Cγ1, Cγ2, Cγ3, V_{L}, and C_{L}.

The present invention includes anti-CD103 antigen-binding fragments and methods of use thereof.

As used herein, a “full length antibody” is, in the case of an IgG, a bivalent molecule comprising two heavy chains and two light chains. Each heavy chain comprises a V_{H} domain followed by a constant domain (C_{H}1), a hinge region, and two more constant (C_{H}2 and C_{H}3) domains; while each light chain comprises one V_{L} domain and one constant (C_{L}) domain. A full length antibody in the case of an IgM is a decavalent or dodecavalent molecule comprising 5 or 6 linked immunoglobulins in which each immunoglobulin monomer has two antigen binding sites formed of a heavy and light chain.

As used herein, unless otherwise indicated, “antibody fragment” or “antigen-binding fragment” refers to antigen-binding fragments of antibodies, i.e. antibody fragments that retain the ability to bind specifically to the antigen bound by the full-length antibody, e.g. fragments that retain one or more CDR regions. Examples of antigen-binding fragments include, but are not limited to, Fab, Fab′, F(ab′)2, and Fv fragments; diabodies; linear antibodies; single-chain antibody molecules, e.g., sc-Fv; nanobodies and multispecific antibodies formed from antibody fragments.

The present invention includes anti-CD103 Fab fragments and methods of use thereof. A “Fab fragment” is comprised of one light chain and the C_{H}1 and variable regions of one heavy chain. The heavy chain of a Fab molecule cannot form a disulfide bond with another heavy chain molecule. A “Fab fragment” can be the product of papain cleavage of an antibody.

The present invention includes anti-CD103 antibodies and antigen-binding fragments thereof which comprise an Fc region and methods of use thereof. An “Fc” region contains two heavy chain fragments comprising the C_{H}3 and C_{H}2 domains of an antibody. The two heavy chain fragments are held together by two or more disulfide bonds and by hydrophobic interactions of the C_{H}3 domains.

The present invention includes anti-CD103 Fab′ fragments and methods of use thereof. A “Fab′ fragment” contains one light chain and a portion or fragment of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)2 molecule.

The present invention includes anti-CD103 F(ab′)₂ fragments and methods of use thereof. A “F(ab′)₂ fragment” contains two light chains and two heavy chains containing a portion of the constant region between the C_{H1} and C_{H2} domains, such that an interchain disulfide bond is formed between the two heavy chains. A F(ab′)₂ fragment thus is composed of two Fab′ fragments that are held together by a disulfide bond between the two heavy chains. An “F(ab′)₂ fragment” can be the product of pepsin cleavage of an antibody.

The present invention includes anti-CD103 Fv fragments and methods of use thereof. The “Fv region” comprises the variable regions from both the heavy and light chains, but lacks the constant regions.

The present invention includes anti-CD103 scFv fragments and methods of use thereof. The term “single-chain Fv” or “scFv” antibody refers to antibody fragments comprising the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen-binding. For a review of scFv, see Pluckthun (1994) THE PHARMACOLOGY OF MONOCLONAL ANTIBODIES, vol. 113, Rosenburg and Moore eds. Springer-Verlag, New York, pp. 269-315. See also, International Patent Application Publication No. WO 88/01649 and U.S. Pat. Nos. 4,946,778 and 5,260,203.

The present invention includes anti-CD103 domain antibodies and methods of use thereof. A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

The present invention includes anti-CD103 bivalent antibodies and methods of use thereof. A “bivalent antibody” comprises two antigen-binding sites. In some instances, the two binding sites have the same antigen specificities. However, bivalent antibodies may be bispecific (see below).

The present invention includes anti-CD103 diabodies and methods of use thereof. As used herein, the term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L} Or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90:6444-6448. Duobodies are described in Labrijn et al., 2013, Proc. Natl. Acad. Sci. USA 110 (13): 5145-5150. For a review of engineered antibody variants generally see Holliger and Hudson (2005) Nat. Biotechnol. 23:1126-1136.

Typically, an antibody or antigen-binding fragment of the invention which is modified in some way retains at least 10% of its binding activity (when compared to the parental antibody) when that activity is expressed on a molar basis. Preferably, an antibody or antigen-binding fragment of the invention retains at least 20%, 50%, 70%, 80%, 90%, 95% or 100% or more of the CD103 binding affinity as the parental antibody. It is also intended that an antibody or antigen-binding fragment of the invention can include conservative or non-conservative amino acid substitutions (referred to as “conservative variants” or “function conserved variants” of the antibody) that do not substantially alter its biologic activity.

The present invention includes isolated anti-CD103 antibodies and antigen-binding fragments thereof and methods of use thereof. Herein, the term “isolated” is not intended to refer to a complete absence of such biological molecules or to an absence of water, buffers, or salts or to components of a pharmaceutical formulation that includes the antibodies or fragments. An “isolated” antibody, antigen-binding fragment, nucleic acid, etc., is one which has been identified and separated and/or recovered from one or more components of its natural environment. In preferred embodiments, the antibody, antigen-binding fragment, nucleic acid, etc., is purified to 75% by weight or more, more preferably to 90% by weight or more, still more preferably to 95% by weight or more, and still more preferably to 98% by weight or more. Thus, “isolated” biological molecules are at least partially free of other biological molecules from the cells or cell cultures in which they are produced. Such biological molecules include nucleic acids, proteins, lipids, carbohydrates, or other material such as cellular debris and growth medium. An isolated antibody or antigen-binding fragment may further be at least partially free of expression system components such as biological molecules from a host cell or of the growth medium thereof.

The present invention includes anti-CD103 chimeric antibodies (e.g., human constant domain/mouse variable domain) and methods of use thereof. As used herein, a “chimeric antibody” is an antibody having the variable domain from a first antibody and the constant domain from a second antibody, where the first and second antibodies are from different species. (U.S. Pat. No. 4,816,567; and Morrison et al., (1984) Proc. Natl. Acad. Sci. USA 81:6851-6855). Typically, the variable domains are obtained from an antibody from an experimental animal (the “parental antibody”), such as a rodent, and the constant domain sequences are obtained from human antibodies, so that the resulting chimeric antibody will be less likely to elicit an adverse immune response in a human subject than the parental (e.g., mouse) antibody.

The present invention includes anti-CD103 humanized antibodies and antigen-binding fragments thereof (e.g., rat or mouse antibodies that have been humanized) and methods of use thereof. As used herein, the term “humanized antibody” refers to forms of antibodies that contain sequences from both human and non-human (e.g., mouse or rat) antibodies. In general, the humanized antibody will comprise substantially of at least one, and typically two, variable domains, in which all or substantially all of the hypervariable loops correspond to those of a non-human immunoglobulin, and all or substantially all of the framework (FR) regions are those of a human immunoglobulin sequence. The humanized antibody may optionally comprise at least a portion of a human immunoglobulin constant region (Fc). For more details about humanized antibodies, see, e.g., Jones et al., Nature, 321:522-525 (1986); Reichmann et al., Nature, 332:323-329 (1988); Presta, Curr. Op. Struct. Biol., 2:593-596 (1992); and Clark, Immunol. Today 21:397-402 (2000).

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, MD; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As used herein, the term “hypervariable region” refers to the amino acid residues of an antibody or antigen-binding fragment thereof that are responsible for antigen-binding. The hypervariable region comprises amino acid residues from a “complementarity determining region” or “CDR” (i.e. CDRL1, CDRL2 and CDRL3 in the light chain variable domain and CDRH1, CDRH2 and CDRH3 in the heavy chain variable domain). See Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196:901-917 (defining the CDR regions of an antibody by structure). As used herein, the term “framework” or “FR” residues refers to those variable domain residues other than the hypervariable region residues defined herein as CDR residues.

“Isolated nucleic acid molecule” or “isolated polynucleotide” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid or polynucleotide is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, but not always, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

As used herein, “germline sequence” refers to a sequence of unrearranged immunoglobulin DNA sequences. Any suitable source of unrearranged immunoglobulin sequences may be used. Human germline sequences may be obtained, for example, from JOINSOLVER germline databases on the website for the National Institute of Arthritis and Musculoskeletal and Skin Diseases of the United States National Institutes of Health. Mouse germline sequences may be obtained, for example, as described in Giudicelli et al. (2005) Nucleic Acids Res. 33: D256-D261.

### Binding Affinity

By way of example, and not limitation, the antibodies disclosed herein may bind human CD103 bivalently with a K_{D} value of 10×10⁻⁹M or lower as determined by surface plasmon resonance (e.g., BIACORE) or a similar technique (e.g. KinExa or bio-layer interferometry (OCTET)). In one embodiment, the antibodies disclosed herein may bind human CD103 bivalently with a K_{D} value of about 5-10×10⁻⁹ M. The Kd value may be determined by surface plasmon resonance (e.g., BIACORE). The Kd value may be determined by similar techniques (e.g. KinExa or OCTET). By way of example, and not limitation, the antigen-binding fragments disclosed herein may bind human CD103 bivalently with a K_{D} value of 10×10⁻⁹ M or lower as determined by surface plasmon resonance (e.g., BIACORE) or a similar technique (e.g. KinExa or bio-layer interferometry (OCTET)). In one embodiment, the antigen-binding fragments disclosed herein may bind human CD103 bivalently with a K_{D} value of about 5-10×10⁻⁹ M. The Kd value may be determined by surface plasmon resonance (e.g., BIACORE). The Kd value may be determined by similar techniques (e.g. KinExa or OCTET). Affinity is calculated as K_{D}=k_{off}/kₒₙ (k_{off} is the dissociation rate constant, Kₒₙ is the association rate constant and K_{D} is the equilibrium constant). Affinity can be determined at equilibrium by measuring the fraction bound (r) of labeled ligand at various concentrations (c). The data are graphed using the Scatchard equation: r/c=K(n−r): where r=moles of bound ligand/mole of receptor at equilibrium; c=free ligand concentration at equilibrium; K=equilibrium association constant; and n=number of ligand binding sites per receptor molecule. By graphical analysis, r/c is plotted on the Y-axis versus r on the X-axis, thus producing a Scatchard plot. Antibody affinity measurement by Scatchard analysis is well known in the art. See, e.g., van Erp et al., J. Immunoassay 12:425-43, 1991; Nelson and Griswold, Comput. Methods Programs Biomed. 27:65-8, 1988.

### Methods of Making Antibodies and Antigen-Binding Fragments Thereof

Thus, the present invention includes methods for making an anti-CD103 antibody or antigen-binding fragment thereof of the present invention comprising culturing a hybridoma cell that expresses the antibody or fragment under condition favorable to such expression and, optionally, isolating the antibody or fragment from the hybridoma and/or the growth medium (e.g. cell culture medium).

The anti-CD103 antibodies disclosed herein may also be produced recombinantly (e.g., in an E. coli/T7 expression system, a mammalian cell expression system or a lower eukaryote expression system). In this embodiment, nucleic acids encoding the antibody immunoglobulin molecules of the invention (e.g., V_{H} or V_{L}) may be inserted into a pET-based plasmid and expressed in the E. coli/T7 system. For example, the present invention includes methods for expressing an antibody or antigen-binding fragment thereof or immunoglobulin chain thereof in a host cell (e.g., bacterial host cell such as E. coli such as BL21 or BL21DE3) comprising expressing T7 RNA polymerase in the cell which also includes a polynucleotide encoding an immunoglobulin chain that is operably linked to a T7 promoter. For example, in an embodiment of the invention, a bacterial host cell, such as a E. coli, includes a polynucleotide encoding the T7 RNA polymerase gene operably linked to a lac promoter and expression of the polymerase and the chain is induced by incubation of the host cell with IPTG (isopropyl-beta-D-thiogalactopyranoside).

There are several methods by which to produce recombinant antibodies which are known in the art. One example of a method for recombinant production of antibodies is disclosed in U.S. Pat. No. 4,816,567.

Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors. Methods of transforming cells are well known in the art. See, for example, U.S. Pat. Nos. 4,399,216; 4,912,040; 4,740,461 and 4,959,455.

Thus, the present invention includes recombinant methods for making an anti-CD103 antibody or antigen-binding fragment thereof of the present invention, or an immunoglobulin chain thereof, comprising introducing a polynucleotide encoding one or more immunoglobulin chains of the antibody or fragment (e.g., heavy and/or light immunoglobulin chain); culturing the host cell (e.g., CHO or Pichia or Pichia pastoris) under condition favorable to such expression and, optionally, isolating the antibody or fragment or chain from the host cell and/or medium in which the host cell is grown.

Anti-CD103 antibodies can also be synthesized by any of the methods set forth in U.S. Pat. No. 6,331,415.

Eukaryotic and prokaryotic host cells, including mammalian cells as hosts for expression of the antibodies or fragments or immunoglobulin chains disclosed herein are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Cell lines of particular preference are selected through determining which cell lines have high expression levels. Other cell lines that may be used are insect cell lines, such as Sf9 cells, amphibian cells, bacterial cells, plant cells and fungal cells. Fungal cells include yeast and filamentous fungus cells including, for example, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. Pichia sp., any Saccharomyces sp., Hansenula polymorpha, any Kluyveromyces sp., Candida albicans, any Aspergillus sp., Trichoderma reesei, Chrysosporium lucknowense, any Fusarium sp., Yarrowia lipolytica, and Neurospora crassa. When recombinant expression vectors encoding the heavy chain or antigen-binding portion or fragment thereof, and/or the light chain or antigen-binding fragment thereof are introduced into mammalian host cells, the antibodies are produced by culturing the host cells for a period of time sufficient to allow for expression of the antibody or fragment or chain in the host cells or secretion into the culture medium in which the host cells are grown.

Antibodies and antigen-binding fragments thereof and immunoglobulin chains can be recovered from the culture medium using standard protein purification methods. Further, expression of antibodies and antigen-binding fragments thereof and immunoglobulin chains of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European U.S. Pat. Nos. 216,846, 0256055, and 0323997 and 0338841. Thus, in an embodiment of the invention, the mammalian host cells (e.g., CHO) lack a glutamine synthetase gene and are grown in the absence of glutamine in the medium wherein, however, the polynucleotide encoding the immunoglobulin chain comprises a glutamine synthetase gene which complements the lack of the gene in the host cell.

The present invention includes methods for purifying an anti-CD103 antibody or antigen-binding fragment thereof of the present invention comprising introducing a sample comprising the antibody or fragment to a purification medium (e.g., cation exchange medium, anion exchange medium, hydrophobic exchange medium, affinity purification medium (e.g., protein-A, protein-G, protein-A/G, protein-L)) and either collecting purified antibody or fragment from the flow-through fraction of said sample that does not bind to the medium; or, discarding the flow-through fraction and eluting bound antibody or fragment from the medium and collecting the eluate. In an embodiment of the invention, the medium is in a column to which the sample is applied. In an embodiment of the invention, the purification method is conducted following recombinant expression of the antibody or fragment in a host cell, e.g., wherein the host cell is first lysed and, optionally, the lysate is purified of insoluble materials prior to purification on a medium.

In general, glycoproteins produced in a particular cell line or transgenic animal will have a glycosylation pattern that is characteristic for glycoproteins produced in the cell line or transgenic animal. Therefore, the particular glycosylation pattern of an antibody will depend on the particular cell line or transgenic animal used to produce the antibody. However, all antibodies encoded by the nucleic acid molecules provided herein, or comprising the amino acid sequences provided herein, comprise the instant invention, independent of the glycosylation pattern the antibodies may have. Similarly, in particular embodiments, antibodies with a glycosylation pattern comprising only non-fucosylated N-glycans may be advantageous, because these antibodies have been shown to typically exhibit more potent efficacy than their fucosylated counterparts both in vitro and in vivo (See for example, Shinkawa et al., J. Biol. Chem. 278:3466-3473 (2003); U.S. Pat. Nos. 6,946,292 and 7,214,775). These antibodies with non-fucosylated N-glycans are not likely to be immunogenic because their carbohydrate structures are a normal component of the population that exists in human serum IgG.

The present invention further includes anti-CD103 antigen-binding fragments of the anti-CD103 antibodies disclosed herein. The antibody fragments include F(ab)₂ fragments, which may be produced by enzymatic cleavage of an IgG by, for example, pepsin. Fab fragments may be produced by, for example, reduction of F(ab)₂ with dithiothreitol or mercaptoethylamine.

Immunoglobulins may be assigned to different classes depending on the amino acid sequences of the constant domain of their heavy chains. In some embodiments, different constant domains may be appended to humanized V_{L} and V_{H} regions derived from the CDRs provided herein. There are at least five major classes of immunoglobulins: IgA, IgD, IgE, IgG and IgM, and several of these may be further divided into subclasses (isotypes), e.g. IgG1, IgG2, IgG3 and IgG4; IgA1 and IgA2. The invention comprises antibodies and antigen-binding fragments of any of these classes or subclasses of antibodies.

In one embodiment, the antibody or antigen-binding fragment comprises a heavy chain constant region, e.g. a human constant region, such as γ1, γ2, γ3, or γ4 human heavy chain constant region or a variant thereof. In another embodiment, the antibody or antigen-binding fragment comprises a light chain constant region, e.g. a human light chain constant region, such as lambda or kappa human light chain region or variant thereof. By way of example, and not limitation the human heavy chain constant region can be γ4 and the human light chain constant region can be kappa. In an alternative embodiment, the Fc region of the antibody is γ4 with a Ser228Pro mutation (Schuurman, J et. al., Mol. Immunol. 38:1-8, 2001).

In one embodiment, the antibody or antigen-binding fragment comprises a heavy chain constant region of the IgG1 subtype. In one embodiment, the antibody or antigen-binding fragment comprises a heavy chain constant region of the IgG2 subtype. In one embodiment, the antibody or antigen-binding fragment comprises a heavy chain constant region of the IgG4 subtype.

### Antibody Engineering

Further included are embodiments in which the anti-CD103 antibodies and antigen-binding fragments thereof are engineered antibodies to include modifications to framework residues within the variable domains the antibody, e.g. to improve the properties of the antibody or fragment. Typically, such framework modifications are made to decrease the immunogenicity of the antibody or fragment. This is usually accomplished by replacing non-CDR residues in the variable domains (i.e. framework residues) in a parental (e.g. rodent) antibody or fragment with analogous residues from the immune repertoire of the species in which the antibody is to be used, e.g. human residues in the case of human therapeutics. Such an antibody or fragment is referred to as a “humanized” antibody or fragment. In some cases, it is desirable to increase the affinity, or alter the specificity of an engineered (e.g. humanized) antibody. One approach is to mutate one or more framework residues to the corresponding germline sequence. More specifically, an antibody or fragment that has undergone somatic mutation can contain framework residues that differ from the germline sequence from which the antibody is derived. Such residues can be identified by comparing the antibody or fragment framework sequences to the germline sequences from which the antibody or fragment is derived. Another approach is to revert to the original parental (e.g., rodent) residue at one or more positions of the engineered (e.g. humanized) antibody, e.g. to restore binding affinity that may have been lost in the process of replacing the framework residues. (See, e.g., U.S. Pat. Nos. 5,693,762, 5,585,089 and 5,530,101).

In certain embodiments, the anti-CD103 antibodies and antigen-binding fragments thereof are engineered (e.g. humanized) to include modifications in the framework and/or CDRs to improve their properties. Such engineered changes can be based on molecular modelling. A molecular model for the variable region for the parental (non-human) antibody sequence can be constructed to understand the structural features of the antibody and used to identify potential regions on the antibody that can interact with the antigen. Conventional CDRs are based on alignment of immunoglobulin sequences and identifying variable regions. Kabat et al., (1991) Sequences of Proteins of Immunological Interest, Kabat, et al.; National Institutes of Health, Bethesda, MD; 5^{th} ed.; NIH Publ. No. 91-3242; Kabat (1978) Adv. Prot. Chem. 32:1-75; Kabat, et al., (1977) J. Biol. Chem. 252:6609-6616. Chothia and coworkers carefully examined conformations of the loops in crystal structures of antibodies and proposed hypervariable loops. Chothia, et al., (1987) J Mol. Biol. 196:901-917 or Chothia, et al., (1989) Nature 342:878-883. There are variations between regions classified as “CDRs” and “hypervariable loops”. Later studies (Raghunathan et al, (2012) J. Mol Recog. 25, 3, 103-113) analyzed several antibody-antigen crystal complexes and observed that the antigen binding regions in antibodies do not necessarily conform strictly to the “CDR” residues or “hypervariable” loops. The molecular model for the variable region of the non-human antibody can be used to guide the selection of regions that can potentially bind to the antigen. In practice the potential antigen binding regions based on the model differ from the conventional “CDR”s or “hypervariable” loops. Commercial scientific software such as Discovery Studio (BIOVIA, Dassault Systems)) can be used for molecular modeling. Human frameworks can be selected based on best matches with the non-human sequence both in the frameworks and in the CDRs. For FR4 (framework 4) in VH, VJ regions for the human germlines are compared with the corresponding non-human region. In the case of FR4 (framework 4) in VL, J-kappa and J-Lambda regions of human germline sequences are compared with the corresponding non-human region. Once suitable human frameworks are identified, the CDRs are grafted into the selected human frameworks. In some cases, certain residues in the VL-VH interface can be retained as in the non-human (parental) sequence. Molecular models can also be used for identifying residues that can potentially alter the CDR conformations and hence binding to antigen. In some cases, these residues are retained as in the non-human (parental) sequence. Molecular models can also be used to identify solvent exposed amino acids that can result in unwanted effects such as glycosylation, deamidation and oxidation. Developability filters can be introduced early on in the design stage to eliminate/minimize these potential problems.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Pat. No. 7,125,689.

In particular embodiments, it will be desirable to change certain amino acids containing exposed side-chains to another amino acid residue in order to provide for greater chemical stability of the final antibody, so as to avoid deamidation or isomerization. The deamidation of asparagine may occur on NG, DG, NG, NS, NA, NT, QG or QS sequences and result in the creation of an isoaspartic acid residue that introduces a kink into the polypeptide chain and decreases its stability (isoaspartic acid effect). Isomerization can occur at DG, DS, DA or DT sequences. In certain embodiments, the antibodies of the present disclosure do not contain deamidation or asparagine isomerism sites.

For example, an asparagine (Asn) residue may be changed to Gln or Ala to reduce the potential for formation of isoaspartate at any Asn-Gly sequences, particularly within a CDR. A similar problem may occur at a Asp-Gly sequence. Reissner and Aswad (2003) Cell. Mol. Life Sci. 60:1281. Isoaspartate formation may debilitate or completely abrogate binding of an antibody to its target antigen. See, Presta (2005) J. Allergy Clin. Immunol. 116:731 at 734. In one embodiment, the asparagine is changed to glutamine (Gln). It may also be desirable to alter an amino acid adjacent to an asparagine (Asn) or glutamine (Gln) residue to reduce the likelihood of deamidation, which occurs at greater rates when small amino acids occur adjacent to asparagine or glutamine. See, Bischoff & Kolbe (1994) J. Chromatog. 662:261. In addition, any methionine residues (typically solvent exposed Met) in CDRs may be changed to Lys, Leu, Ala, or Phe or other amino acids in order to reduce the possibility that the methionine sulfur would oxidize, which could reduce antigen-binding affinity and also contribute to molecular heterogeneity in the final antibody preparation. Id. Additionally, in order to prevent or minimize potential scissile Asn-Pro peptide bonds, it may be desirable to alter any Asn-Pro combinations found in a CDR to Gln-Pro, Ala-Pro, or Asn-Ala. Antibodies with such substitutions are subsequently screened to ensure that the substitutions do not decrease the affinity or specificity of the antibody for CD103, or other desired biological activity to unacceptable levels.

TABLE 2
Exemplary stabilizing CDR variants

| CDR Residue | Stabilizing Variant Sequence |
|---|---|
| Asn-Gly | Gln-Gly, Ala-Gly, or Asn-Ala |
| (N-G) | (Q-G), (A-G), or (N-A) |
| Asp-Gly | Glu-Gly, Ala-Gly or Asp-Ala |
| (D-G) | (E-G), (A-G), or (D-A) |
| Met | Lys, Leu, Ala, or Phe |
| (M) | (K),(L), (A), or (F) |
| Asn | Gln or Ala |
| (N) | (Q) or (A) |
| Asn-Pro | Gln-Pro, Ala-Pro, or Asn-Ala |
| (N-P) | (Q-P), (A-P), or (N-A) |

Another type of framework modification involves mutating one or more residues within the framework regions to prevent aggregation. The risk of an antibody to aggregate can be assessed using the spatial aggregation propensity-See, Chennamsetty, N et al (2010) J. Phys. Chem. 114, 6614-6624. The method requires the calculation of the Solvent Accessible Area (SAA) for each atom. The molecular aggregation score is then calculated as the sum of all atomic scores. For a given radius and size of molecule, this is an approximate indication of its overall tendency to aggregate. Residues with a high aggregation score are replaced by residues with a lower score (e.g. more hydrophilic amino acids).

### Antibody Engineering of the Fc Region

The antibodies (e.g., humanized antibodies) and antigen-binding fragments thereof disclosed herein can also be engineered to include modifications within the Fc region, typically to alter one or more properties of the antibody, such as serum half-life, complement fixation, Fc receptor binding, and/or effector function (e.g., antigen-dependent cellular cytotoxicity). Furthermore, the antibodies and antigen-binding fragments thereof disclosed herein can be chemically modified (e.g., one or more chemical moieties can be attached to the antibody) or be modified to alter its glycosylation, again to alter one or more properties of the antibody or fragment. Each of these embodiments is described in further detail below. The numbering of residues in the Fc region is that of the EU index of Kabat.

The antibodies and antigen-binding fragments thereof disclosed herein also include antibodies and fragments with modified (or blocked) Fc regions to provide altered effector functions. See, e.g., U.S. Pat. No. 5,624,821; WO2003/086310; WO2005/120571; WO2006/0057702. Such modifications can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc regions. Changes to the Fc can also alter the half-life of antibodies in therapeutic antibodies, enabling less frequent dosing and thus increased convenience and decreased use of material. See Presta (2005) J. Allergy Clin. Immunol. 116:731 at 734-35.

In one embodiment, the antibody or antigen-binding fragment of the invention is an IgG4 isotype antibody or fragment comprising a Serine to Proline mutation at a position corresponding to position 228 (S228P; EU index; SEQ ID NO: 66) in the hinge region of the heavy chain constant region. This mutation has been reported to abolish the heterogeneity of inter-heavy chain disulfide bridges in the hinge region (Angal et al (1993). Mol. Immunol. 30:105-108; position 241 is based on the Kabat numbering system).

In one embodiment of the invention, the hinge region of C_{H}1 is modified such that the number of cysteine residues in the hinge region is increased or decreased. This approach is described further in U.S. Pat. No. 5,677,425. The number of cysteine residues in the hinge region of CH1 is altered, for example, to facilitate assembly of the light and heavy chains or to increase or decrease the stability of the antibody.

In another embodiment, the Fc hinge region of an antibody or antigen-binding fragment of the invention is mutated to decrease the biological half-life of the antibody or fragment. More specifically, one or more amino acid mutations are introduced into the CH2-CH3 domain interface region of the Fc-hinge fragment such that the antibody or fragment has impaired Staphylococcyl protein A (SpA) binding relative to native Fc-hinge domain SpA binding. This approach is described in further detail in U.S. Pat. No. 6,165,745.

In another embodiment, the antibody or antigen-binding fragment of the invention is modified to increase its biological half-life. Various approaches are possible. For example, one or more of the following mutations can be introduced: T252L, T254S, T256F, as described in U.S. Pat. No. 6,277,375. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022.

In yet other embodiments, the Fc region is altered by replacing at least one amino acid residue with a different amino acid residue to alter the effector function(s) of the antibody or antigen-binding fragment. For example, one or more amino acids selected from amino acid residues 234, 235, 236, 237, 297, 318, 320 and 322 can be replaced with a different amino acid residue such that the antibody has an altered affinity for an effector ligand and retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260.

In another example, one or more amino acids selected from amino acid residues 329, 331 and 322 can be replaced with a different amino acid residue such that the antibody has altered Clq binding and/or reduced or abolished complement dependent cytotoxicity (CDC). This approach is described in further detail in U.S. Pat. No. 6,194,551.

In another example, one or more amino acid residues within amino acid positions 231 and 239 are altered to thereby alter the ability of the antibody to fix complement. This approach is described further in PCT Publication WO 94/29351.

The proteins of the invention, which are preferably antibodies and most preferably IgG antibodies or fragments thereof, may have altered (e.g., relative to an unmodified antibody) FcγR binding properties (examples of binding properties include but are not limited to, binding specificity, equilibrium dissociation constant (K_{D}), dissociation and association rates (k_{off} and kₒₙ respectively), binding affinity and/or avidity) and that certain alterations are more or less desirable. It is known in the art that the equilibrium dissociation constant (K_{D}) is defined as k_{off}/kₒₙ, and Kₐ is the reciprocal of K_{D}.

The affinities and binding properties of an Fc region for its ligand, may be determined by a variety of in vitro assay methods (biochemical or immunological based assays) known in the art for determining Fc-FcγR interactions, i.e., specific binding of an Fc region to an FcγR including but not limited to, equilibrium methods (e.g., enzyme-linked immuno absorbent assay (ELISA) or radioimmunoassay (RIA)), or kinetics (e.g. BIACORE®, Octet®, or KinExa® analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels.

In certain embodiments, the proteins of the present invention bind to one or more human FcγRs. In certain embodiments, the proteins of the present invention bind to one or more human FcγRs selected from the group consisting of FcγRI, FcγRIIB, FcγRIIC, FcγRIIIA-F158, and FcγRIIIA-V158 with an affinity at least 10-fold, preferably at least 30-fold, and more preferably at least 100-fold, less than equivalent protein having a wild-type human IgG1 heavy chain constant domain (SEQ ID NO: 119) Fc region. In certain embodiments, the proteins of the present invention bind human FcγRI with an affinity at least 10-fold, preferably at least 30-fold, and more preferably at least 100-fold, less than equivalent protein having a wild-type human IgG1 heavy chain constant domain (SEQ ID NO: 119) Fc region. In certain embodiments, the proteins of the present invention bind human FcγRIIB with an affinity at least 10-fold, preferably at least 30-fold, and more preferably at least 100-fold, less than equivalent protein having a wild-type human IgG1 heavy chain constant domain (SEQ ID NO: 119) Fc region. In certain embodiments, the proteins of the present invention bind human FcγRIIC with an affinity at least 10-fold, preferably at least 30-fold, and more preferably at least 100-fold, less than equivalent protein having a wild-type human IgG1 heavy chain constant domain (SEQ ID NO: 119) Fc region. In certain embodiments, the proteins of the present invention bind human FcγRIIIA-F158 with an affinity at least 10-fold, preferably at least 30-fold, and more preferably at least 100-fold, less than equivalent protein having a wild-type human IgG1 heavy chain constant domain (SEQ ID NO: 119) Fc region. In certain embodiments, the proteins of the present invention bind human FcγRIIIA-V158 with an affinity at least 10-fold, preferably at least 30-fold, and more preferably at least 100-fold, less than equivalent protein having a wild-type human IgG1 heavy chain constant domain (SEQ ID NO: 119). Fc region. In certain embodiments, the proteins of the present invention bind to one or more human FcγRs selected from the group consisting of FcγRI, FcγRIIB, FcγRIIC, FcγRIIIA-F158, and FcγRIIIA-V158 with an affinity at least 10-fold, preferably at least 30-fold, and more preferably at least 100-fold, less than equivalent protein having a wild-type human IgG4 heavy chain constant domain (SEQ ID NO: 66) Fc region. In certain embodiments, the proteins of the present invention bind human FcγRI with an affinity at least 10-fold, preferably at least 30-fold, and more preferably at least 100-fold, less than equivalent protein having a wild-type human IgG4 heavy chain constant domain (SEQ ID NO: 66) Fc region. Fc region. In certain embodiments, the proteins of the present invention bind human FcγRIIB with an affinity at least 10-fold, preferably at least 30-fold, and more preferably at least 100-fold, less than equivalent protein having a wild-type human IgG4 heavy chain constant domain (SEQ ID NO: 66) Fc region. In certain embodiments, the proteins of the present invention bind human FcγRIIC with an affinity at least 10-fold, preferably at least 30-fold, and more preferably at least 100-fold, less than equivalent protein having a wild-type human IgG4 heavy chain constant domain (SEQ ID NO: 66) Fc region. In certain embodiments, the proteins of the present invention bind human FcγRIIIA-F158 with an affinity at least 10-fold, preferably at least 30-fold, and more preferably at least 100-fold, less than equivalent protein having a wild-type human IgG4 heavy chain constant domain (SEQ ID NO: 66) Fc region. In certain embodiments, the proteins of the present invention bind human FcγRIIIA-V158 with an affinity at least 10-fold, preferably at least 30-fold, and more preferably at least 100-fold, less than equivalent protein having a wild-type human IgG4 heavy chain constant domain (SEQ ID NO: 66) Fc region.

In various embodiments, the proteins of the invention comprise an immunoglobulin Fc region comprising an immunoglobulin C2 region and an immunoglobulin C3 region and an immunoglobulin hinge region. By way of example, the immunoglobulin Fc region may be an IgG Fc region, an IgE Fc region, or an IgA Fc region. In certain preferred embodiments, the protein comprises two immunoglobulin Fc regions, each immunoglobulin Fc region comprising an immunoglobulin C2 region and an immunoglobulin C3 region and an immunoglobulin hinge region, wherein the hinge region of one of the immunoglobulin Fc regions is bound to the hinge region of the other immunoglobulin Fc region to form a dimeric Fc structure. Most preferably, such a protein is a human or humanized IgG protein.

In certain embodiments, the proteins of the invention comprise a mutated IgG4 Fc region, and preferably the protein is an IgG comprising two mutated IgG4 Fc regions to form a dimeric Fc structure. By way of example, a mutated IgG4 Fc region may comprise one of the mutations, or mutational combinations, recited in Table 3. The numbering system of the constant region referred to in this table is that of the EU index as set forth in Kabat et al. (1991, NIH Publication 91-3242, National Technical Information Service, Springfield, VA). In the table, the first letter and number represent the unmodified amino acid and its position and the second letter represents the substituted amino acid at said position. For those entries that include combinations of more than one mutation, each mutation in the combination is separated by a “/”. Deletions are indicated by “Δ

TABLE 3

| | | |
|---|---|---|
| N297Q | L235E | N297Q/L235E |
| F234A | Q268A | F234A/L235A/G23A/ |
| | | P238A |
| F234A/L235A/AG236/ | F234A/L235A/G237A/ | F234A/L235A/ΔG236/ |
| G237A/P238A | P238A/Q268A | G237A/P238A/Q268A |
| F234A/L235A | L235E/P329G | L235A/G237A/E318A |
| F234A/L235A/G237A/ | F234A/L235A/ΔG236/ | F234A/L235A/G237A/ |
| P238S | G237A/P238S | P238S/Q268A |
| F234A/L235A/ΔG236/ | | |
| G237A/P238S/Q268A | | |

In certain embodiments, the proteins of the invention comprise a mutated IgG1 Fc region, and preferably the protein is an IgG comprising two mutated IgG1 Fc regions to form a dimeric Fc structure. By way of example, a mutated IgG1 Fc region may comprise one of the mutations recited in Table 4. The numbering system of the constant region referred to in this table is that of the EU index as set forth in Kabat et al. (1991, NIH Publication 91-3242, National Technical Information Service, Springfield, VA). In the table, the first letter and number represent the unmodified amino acid and its position and the second letter represents the substituted amino acid at said position.

TABLE 4

| | | |
|---|---|---|
| K222Y | P232K | A231K |
| E233N | E233Q | E233R |
| E233S | E233T | E233H |
| E233A | E233V | E233L |
| E233F | E233M | E233Y |
| E233W | E233G | L234D |
| L234E | L234N | L234Q |
| L234T | L234H | L234F |
| L234K | L234R | L234S |
| L234A | L234M | L234V |
| L235E | L235T | L235F |
| L235K | L235R | L235A |
| L235M | L235W | L235N |
| L235Q | L235H | L235V |
| G236A | G236N | G236R |
| G236H | G236L | G236F |
| G236P | G237A | G237E |
| G237N | G237Q | G237K |
| G237R | G237S | G237T |
| G237H | G237L | G237I |
| G237F | G237M | G237Y |
| G237P | P238K | P238N |
| P238R | P238S | P238T |
| P238Y | P238G | P238A |
| S239A | S239N | S239F |
| S239K | S239R | S239V |
| S239W | S239P | S239H |
| S239Y | D249H | V240A |
| F241W | F241L | F243W |
| F243L | F243E | P244H |
| P245A | P247V | P247G |
| V253I | V263I | V263T |
| V263M | V264D | V264E |
| V264K | V264F | V264M |
| V264H | V264W | V264G |
| V264Q | V264A | V264L |
| D265A | D265E | D265Q |
| D265S | D265H | D265V |
| D265L | D265F | D265M |
| D265Y | D265N | D265G |
| V266T | V266M | V266A |
| S267G | S267H | S267N |
| S267P | S267R | S267T |
| S267F | S267W | E269A |
| E269K | E269S | E269V |
| E269F | E269I | E269M |
| E269W | E269H | E269T |
| E269L | E269N | E269Y |
| E269R | E269P | E269G |
| D270A | D270N | D270E |
| D270Q | D270T | D270H |
| D270R | D270S | D270L |
| D270I | D270F | D270W |
| D270P | D270G | P271H |
| P271Q | P271K | P271R |
| P271S | P271V | P271F |
| P271W | D280L | D280W |
| D280P | E293F | E294A |
| E293Y | E294K | E294R |
| E294S | E294V | E294L |
| E294F | Q295A | Q295W |
| Q295P | Q295G | Y296E |
| Y296Q | Y296D | Y296N |
| Y296S | Y296T | Y296L |
| Y296I | Y296A | Y296V |
| Y296M | N297S | N297D |
| N297Q | N297A | S298T |
| S298N | S298K | S298R |
| T299A | T299H | T299D |
| T299E | T299N | T299Q |
| T299K | T299R | T299I |
| T299F | T299M | T299Y |
| T299W | T299S | T299V |
| T299P | T299G | Y300E |
| Y300K | Y300R | Y300S |
| Y300P | Y300W | V303A |
| V303D | W313F | E318A |
| E318V | E318Q | E318H |
| E318L | E318Y | K320A |
| K322A | K322E | N325A |
| N325V | N325H | N325K |
| N325Y | N325W | N325P |
| N325G | N325Q | N325D |
| N325E | N325L | N325I |
| A327Q | A327E | A327N |
| A327L | A327I | A327F |
| A327W | L328N | L328F |
| L328H | L328R | L328T |
| L328V | L328I | L328P |
| L328M | L328E | L328A |
| P329A | P329F | P329D |
| P329N | P329Q | P329K |
| P329S | P329T | P329H |
| P329V | P329L | P329M |
| P329Y | P329W | P329G |
| P329R | A330L | A330R |
| A330P | A330T | A330V |
| A330F | A330H | P331A |
| P331S | P331N | P331E |
| I332K | I332N | I332Q |
| I332T | I332H | I332Y |
| I332A | I332R | E333N |
| E333R | I336E | I336Y |
| S337H | | |

In certain embodiments, a mutated IgG1 Fc region may comprise one of the mutational combinations recited in Table 5. The numbering system of the constant region referred to in this table is that of the EU index as set forth in Kabat et al. (1991, NIH Publication 91-3242, National Technical Information Service, Springfield, VA). In the table, the first letter and number represent the unmodified amino acid and its position and the second letter represents the substituted amino acid at said position. For each of the combinations of more than one mutation, each mutation in the combination is separated by a “/” and deletions are indicated by a “Δ”.

TABLE 5

| | | |
|---|---|---|
| C220S/C226S/ | C226S/C229S/E233P/ | E233P/L234V/L235A |
| C229S/P238S | L234V/L235A | |
| E233P/L234V/ | E233P/L234V/L235A/ΔG236/ | L234A/L235A |
| L235A/ΔG236 | A327G/A330S/P331S | |
| L235A/G237A | L235A/G237A/E318S/ | L235A/G237A/P331A |
| | K320S/K322S | |
| L234F/L235E | L234F/L235E/D265A | L234F/L235E/D265A/ |
| | | N297Q/P331S |
| L234F/L235E/N297Q | L234F/L235E/P329G | L234F/L235A/K322Q/ |
| | | M252Y/S254T/T256E |
| L234F/L235Q/K322Q/ | L234F/L235Q/P331G/M252Y/ | G236R/L328R |
| M252Y/S254T/T256E | S254T/T256E | |
| S239D/D265I/N297D/I332E | S239D/D265L/N297D/I332E | S239D/D265F/N297D/I332E |
| S239D/D265Y/N297D/I332E | S239D/D265T/N297D/I332E | S239D/N297D/A330Y/I332E |
| S239D/F241S/F243H/V262T/ | V264E/N297D/I332E | D265A/P331S |
| V264T/N297D/K326E/I332E | | |
| D265A/N297Q | N297D/D265Y/T299L/I332E | N297D/D265Y/I332E |
| N297D/I332E/Y296D | N297D/I332E | N297D/I332E/Y296E |
| N297D/I332E/Y296N | N297D/I332E/Y296Q | N297D/I332E/Y296H |
| N297D/I332E/Y296T | N297D/I332E/T299V | N297D/I332E/T299I |
| N297D/I332E/T299L | N297D/I332E/T299F | N297D/I332E/T299H |
| N297D/I332E/T299E | N297D/I332E/A330Y | N297D/I332E/S298A/A330Y |
| N297E/D265F/I332E | N297E/I332E | F241E/F243R/V262E/ |
| | | V264R |
| F241E/F243Q/V262T/V264E | F241L/F243L/V262I/V264I | F241W/F243W |
| F241W/F243W/V262A/V264A | F241L/V262I | F243L/V262I/V264W |
| F241Y/F243Y/V262T/V264T | F241E/F243R/V262E/V264R | F241E/F243Q/V262T/V264E |
| F241R/F243Q/V262T/V264R | F241E/F243Y/V262T/V264R | P244H/P245A/P247V |
| F241E/F243R/V262E/V264R/ | F241E/F243Y/V262T/V264R | F241E/F243Y/V262T/ |
| I332E | | V264R/I332E |
| S239E/D265G | S239E/D265N | S239E/D265Q |
| M252Y/S254T/T256E | S267Q/A327S | S267L/A327S |
| N297S/I332E | S239N/I332N | S239N/I332Q |
| S239Q/I332N | S239Q/I332Q | S298N/Y300S |
| S298N/T299A/Y300S | N297Q/S298N/Y300S | E318S/K320S/K322S |
| E318S/K320S/K322S/P311A | L328E/I332E | L328N/I332E |
| L234A/L235A/G237A/P238A/ | L234A/L235A/G237A/P238S/ | L234A/L235A/G237A/P238A/ |
| H268A/A330S/P331S | H268A/A330S/P331S | H268A/A330S/P331S |
| L328Q/I332E | L328H/I332E | |

In certain embodiments, the proteins of the invention comprise a wild type or mutated IgG2 Fc region, and preferably the protein is an IgG comprising two wild type or mutated IgG2 Fc regions to form a dimeric Fc structure. A mutated IgG2 Fc region may comprise one of the mutations, or mutational combinations, recited in Table 6. The numbering system of the constant region referred to in this table is that of the EU index as set forth in Kabat et al. (1991, NIH Publication 91-3242, National Technical Information Service, Springfield, VA). In the table, the first letter and number represent the unmodified amino acid and its position and the second letter represents the substituted amino acid at said position. For those entries that include combinations of more than one mutation, each mutation in the combination is separated by a “/”.

TABLE 6

| | | |
|---|---|---|
| V234A | G237A | A235E/G237A |
| V234A/A235E/G237A | V234A/G237A | V234A/G237A/P238S |
| H268Q/V309L/ | V234A/G237A/H268A/ | V234A/G237A/H268QA/ |
| A330S/P331S | V309L/A330S/P331S | V309L/A330S/P331S |
| V234A/G237A/P238S/H268A/ | P233S/V234A/G237A/P238S | P233S/V234A/G237A/H268A/ |
| V309L/A330S/P331S | | V309L/A330S/P331S |
| P233S/V234A/G237A/H268Q/ | P233S/V234A/G237A/P238S/ | |
| V309L/A330S/P331S | H268A/V309L/A330S/P331S | |

### Production of Antibodies with Modified Glycosylation

In still another embodiment, the antibodies or antigen-binding fragments of the invention comprise a particular glycosylation pattern. For example, an afucosylated or an aglycosylated antibody or fragment can be made (i.e., the antibody lacks fucose or glycosylation, respectively). The glycosylation pattern of an antibody or fragment may be altered to, for example, increase the affinity or avidity of the antibody or fragment for a CD103 antigen. Such modifications can be accomplished by, for example, altering one or more of the glycosylation sites within the antibody or fragment sequence. For example, one or more amino acid substitutions can be made that result in removal of one or more of the variable region framework glycosylation sites to thereby eliminate glycosylation at that site. Such deglycosylation may increase the affinity or avidity of the antibody or fragment for antigen. See, e.g., U.S. Pat. Nos. 5,714,350 and 6,350,861.

Antibodies and antigen-binding fragments disclosed herein may further include those produced in lower eukaryote host cells, in particular fungal host cells such as yeast and filamentous fungi have been genetically engineered to produce glycoproteins that have mammalian- or human-like glycosylation patterns (See for example, Choi et al, (2003) Proc. Natl. Acad. Sci. 100:5022-5027; Hamilton et al., (2003) Science 301:1244-1246; Hamilton et al., (2006) Science 313:1441-1443; Nett et al., Yeast 28 (3): 237-52 (2011); Hamilton et al., Curr Opin Biotechnol. 18 (5): 387-92 (2007)). A particular advantage of these genetically modified host cells over currently used mammalian cell lines is the ability to control the glycosylation profile of glycoproteins that are produced in the cells such that compositions of glycoproteins can be produced wherein a particular N-glycan structure predominates (see, e.g., U.S. Pat. Nos. 7,029,872 and 7,449,308). These genetically modified host cells have been used to produce antibodies that have predominantly particular N-glycan structures (See for example, Li et al., (2006) Nat. Biotechnol. 24:210-215).

In particular embodiments, the antibodies and antigen-binding fragments thereof disclosed herein further include those produced in lower eukaryotic host cells and which comprise fucosylated and non-fucosylated hybrid and complex N-glycans, including bisected and multiantennary species, including but not limited to N-glycans such as GlcNAc₍₁₋₄₎Man₃GlcNAc₂; Gal₍₁₋₄₎GlcNAc₍₁₋₄₎Man₃GlcNAc₂; NANA₍₁₋₄₎Gal₍₁₋₄₎GlcNAc₍₁₋₄₎Man₃GlcNAc₂.

In particular embodiments, the antibodies and antigen-binding fragments thereof provided herein may comprise antibodies or fragments having at least one hybrid N-glycan selected from the group consisting of GlcNAcMansGlcNAc₂; GalGlcNAcMan₅GlcNAc₂; and NANAGalGlcNAcMan₅GlcNAc₂. In particular aspects, the hybrid N-glycan is the predominant N-glycan species in the composition.

In particular embodiments, the antibodies and antigen-binding fragments thereof provided herein comprise antibodies and fragments having at least one complex N-glycan selected from the group consisting of GlcNAcMan₃GlcNAc₂; GalGlcNAcMan₃GlcNAc₂; NANAGalGlcNAcMan₃GlcNAc₂; GlcNAC₂Man₃GlcNAC₂; GalGlcNAc₂Man₃GlcNAc₂; Gal₂GlcNAc₂Man₃GlcNAc₂; NANAGal₂GlcNAc₂Man₃GlcNAc₂; and NANA₂Gal₂GlcNAc₂Man₃GlcNAc₂. In particular aspects, the complex N-glycan are the predominant N-glycan species in the composition. In further aspects, the complex N-glycan is a particular N-glycan species that comprises about 30%, 40%, 50%, 60%, 70%, 80%, 90%, 95%, 97%, 98%, 99%, or 100% of the complex N-glycans in the composition. In one embodiment, the antibody and antigen binding fragments thereof provided herein comprise complex N-glycans, wherein at least 50%, 60%, 70%, 80%, 90%, 95%, 97%, 98%, 99%, or 100% of the complex N-glycans comprise the structure NANA₂Gal₂GlcNAc₂Man₃GlcNAc₂, wherein such structure is afucosylated. Such structures can be produced, e.g., in engineered Pichia pastoris host cells.

In particular embodiments, the N-glycan is fucosylated. In general, the fucose is in an α1,3-linkage with the GlcNAc at the reducing end of the N-glycan, an α1,6-linkage with the GlcNAc at the reducing end of the N-glycan, an α1,2-linkage with the Gal at the non-reducing end of the N-glycan, an α1,3-linkage with the GlcNac at the non-reducing end of the N-glycan, or an α1,4-linkage with a GlcNAc at the non-reducing end of the N-glycan.

Therefore, in particular aspects of the above the glycoprotein compositions, the glycoform is in an α1,3-linkage or α1,6-linkage fucose to produce a glycoform selected from the group consisting of Man₅GlcNAC₂(Fuc), GlcNAcMansGlcNAc₂(Fuc), Man₃GlcNAC₂(Fuc), GlcNAcMan₃GlcNAc₂(Fuc), GlcNAc₂Man₃GlcNAc₂(Fuc), GalGlcNAC₂Man₃GlcNAc₂(Fuc), Gal₂GlcNAc₂Man₃GlcNAc₂(Fuc), NANAGal₂GlcNAc₂Man₃GlcNAC₂(Fuc), and NANA₂Gal₂GlcNAc₂Man₃GlcNAc₂(Fuc); in an α1,3-linkage or α1,4-linkage fucose to produce a glycoform selected from the group consisting of GlcNAc(Fuc)MansGlcNAc₂, GlcNAc(Fuc)Man₃GlcNAc₂, GlcNAc₂(Fuc₁₋₂)Man₃GlcNAc₂, GalGlcNAC₂(Fuc₁₋₂)Man₃GlcNAc₂, Gal₂GlcNAc₂(Fuc1-2) Man3GlcNAc2, NANAGal2GlcNAc2(Fuc₁₋₂)Man₃GlcNAc₂, and NANA₂Gal₂GlcNAc₂(Fuc₁₋₂)Man₃GlcNAc₂; or in an α1,2-linkage fucose to produce a glycoform selected from the group consisting of Gal(Fuc)GlcNAc₂Man₃GlcNAc₂, Gal₂(Fuc₁₋₂)GlcNAc₂Man₃GlcNAc₂, NANAGal2(Fuc₁₋₂)GlcNAc₂Man₃GlcNAc₂, and NANA2Gal2(Fuc₁₋₂)GlcNAc₂Man₃GlcNAc₂.

In further aspects, the antibodies (e.g., humanized antibodies) or antigen-binding fragments thereof comprise high mannose N-glycans, including but not limited to, Man₈GlcNAC₂, Man₇GlcNAc₂, Man₆GlcNAc₂, MansGlcNAc₂, Man₄GlcNAc₂, or N-glycans that consist of the Man₃GlcNAc₂ N-glycan structure.

In further aspects of the above, the complex N-glycans further include fucosylated and non-fucosylated bisected and multiantennary species.

As used herein, the terms “N-glycan” and “glycoform” are used interchangeably and refer to an N-linked oligosaccharide, for example, one that is attached by an asparagine-N-acetylglucosamine linkage to an asparagine residue of a polypeptide. N-linked glycoproteins contain an N-acetylglucosamine residue linked to the amide nitrogen of an asparagine residue in the protein. The predominant sugars found on glycoproteins are glucose, galactose, mannose, fucose, N-acetylgalactosamine (GalNAc), N-acetylglucosamine (GlcNAc) and sialic acid (e.g., N-acetyl-neuraminic acid (NANA)). The processing of the sugar groups occurs co-translationally in the lumen of the ER and continues post-translationally in the Golgi apparatus for N-linked glycoproteins.

N-glycans have a common pentasaccharide core of Man₃GlcNAC₂ (“Man” refers to mannose; “Glc” refers to glucose; and “NAc” refers to N-acetyl; GlcNAc refers to N-acetylglucosamine). Usually, N-glycan structures are presented with the non-reducing end to the left and the reducing end to the right. The reducing end of the N-glycan is the end that is attached to the Asn residue comprising the glycosylation site on the protein. N-glycans differ with respect to the number of branches (antennae) comprising peripheral sugars (e.g., GlcNAc, galactose, fucose and sialic acid) that are added to the Man₃GlcNAC₂ (“Man₃”) core structure which is also referred to as the “trimannose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). Complex N-glycans may also have multiple antennae on the “trimannose core,” often referred to as “multiple antennary glycans.” A “hybrid” N-glycan has at least one GlcNAc on the terminal of the 1,3 mannose arm of the trimannose core and zero or more mannoses on the 1,6 mannose arm of the trimannose core. The various N-glycans are also referred to as “glycoforms.”

With respect to complex N-glycans, the terms “G-2”, “G-1”, “G0”, “G1”, “G2”, “A1”, and “A2” mean the following. “G-2” refers to an N-glycan structure that can be characterized as Man₃GlcNAc₂; the term “G-1” refers to an N-glycan structure that can be characterized as GlcNAcMan₃GlcNAc₂; the term “GO” refers to an N-glycan structure that can be characterized as GlcNAc₂Man₃GlcNAc₂; the term “G1” refers to an N-glycan structure that can be characterized as GalGlcNAc2Man₃GlcNAc₂; the term “G2” refers to an N-glycan structure that can be characterized as Gal2GlcNAc₂Man₃GlcNAc₂; the term “A1” refers to an N-glycan structure that can be characterized as NANAGal2GlcNAc₂Man₃GlcNAc₂; and, the term “A2” refers to an N-glycan structure that can be characterized as NANA2Gal2GlcNAc₂Man₃GlcNAc₂. Unless otherwise indicated, the terms G-2″, “G-1”, “G0”, “G1”, “G2”, “A1”, and “A2” refer to N-glycan species that lack fucose attached to the GlcNAc residue at the reducing end of the N-glycan. When the term includes an “F”, the “F” indicates that the N-glycan species contains a fucose residue on the GlcNAc residue at the reducing end of the N-glycan. For example, G0F, G1F, G2F, A1F, and A2F all indicate that the N-glycan further includes a fucose residue attached to the GlcNAc residue at the reducing end of the N-glycan. Lower eukaryotes such as yeast and filamentous fungi do not normally produce N-glycans that produce fucose.

With respect to multiantennary N-glycans, the term “multiantennary N-glycan” refers to N-glycans that further comprise a GlcNAc residue on the mannose residue comprising the non-reducing end of the 1,6 arm or the 1,3 arm of the N-glycan or a GlcNAc residue on each of the mannose residues comprising the non-reducing end of the 1,6 arm and the 1,3 arm of the N-glycan. Thus, multiantennary N-glycans can be characterized by the formulas GlcNAc₍₂₋₄₎Man₃GlcNAc₂, Gal₍₁₋₄₎GlcNAc₍₂₋₄₎Man₃GlcNAc₂, or NANA₍₁₋₄₎Gal₍₁₋₄₎GlcNAc₍₂₋₄₎Man₃GlcNAc₂. The term “1-4” refers to 1, 2, 3, or 4 residues.

With respect to bisected N-glycans, the term “bisected N-glycan” refers to N-glycans in which a GlcNAc residue is linked to the mannose residue at the reducing end of the N-glycan. A bisected N-glycan can be characterized by the formula GlcNAc₃Man₃GlcNAc₂ wherein each mannose residue is linked at its non-reducing end to a GlcNAc residue. In contrast, when a multiantennary N-glycan is characterized as GlcNAc₃Man₃GlcNAc₂, the formula indicates that two GlcNAc residues are linked to the mannose residue at the non-reducing end of one of the two arms of the N-glycans and one GlcNAc residue is linked to the mannose residue at the non-reducing end of the other arm of the N-glycan.

In certain embodiments, the proteins of the invention comprise an aglycosylated Fc region. By way of example, an IgG1 Fc region may be aglycosylayed by deleting or substituting residue N297.

### Antibody Physical Properties

The antibodies and antigen-binding fragments thereof disclosed herein may further contain one or more glycosylation sites in either the light or heavy chain immunoglobulin variable region. Such glycosylation sites may result in increased immunogenicity of the antibody or fragment or an alteration of the pK of the antibody due to altered antigen-binding (Marshall et al. (1972) Annu Rev Biochem 41:673-702; Gala and Morrison (2004) J Immunol 172:5489-94; Wallick et al (1988) J Exp Med 168:1099-109; Spiro (2002) Glycobiology 12: 43R-56R; Parekh et al (1985) Nature 316:452-7; Mimura et al. (2000) Mol Immunol 37:697-706). Glycosylation has been known to occur at motifs containing an N-X-S/T sequence.

Each antibody or antigen-binding fragment will have a unique isoelectric point (pI), which generally falls in the pH range between 6 and 9.5. The pI for an IgG1 antibody typically falls within the pH range of 7-9.5 and the pI for an IgG4 antibody typically falls within the pH range of 6-8.

Each antibody or antigen-binding fragment will have a characteristic melting temperature, with a higher melting temperature indicating greater overall stability in vivo (Krishnamurthy R and Manning M C (2002) Curr Pharm Biotechnol 3:361-71). In general, the T_{M1} (the temperature of initial unfolding) may be greater than 60° C., greater than 65° C., or greater than 70° C. The melting point of an antibody or fragment can be measured using differential scanning calorimetry (Chen et al (2003) Pharm Res 20:1952-60; Ghirlando et al (1999) Immunol Lett 68:47-52) or circular dichroism (Murray et al. (2002) J. Chromatogr Sci 40:343-9).

In a further embodiment, antibodies and antigen-binding fragments thereof are selected that do not degrade rapidly. Degradation of an antibody or fragment can be measured using capillary electrophoresis (CE) and MALDI-MS (Alexander A J and Hughes D E (1995) Anal Chem 67:3626-32).

In a further embodiment, antibodies and antigen-binding fragments thereof are selected that have minimal aggregation effects, which can lead to the triggering of an unwanted immune response and/or altered or unfavorable pharmacokinetic properties. Generally, antibodies and fragments are acceptable with aggregation of 25% or less, 20% or less, 15% or less, 10% or less, or 5% or less. Aggregation can be measured by several techniques, including size-exclusion column (SEC), high performance liquid chromatography (HPLC), and light scattering.

### Antibody Conjugates

The anti-CD103 antibodies disclosed herein may also be conjugated to a chemical moiety. The anti-CD103 antigen-binding fragments disclosed herein may also be conjugated to a chemical moiety. The chemical moiety may be, inter alia, a polymer. The chemical moiety may be, inter alia, a radionucleotide. The chemical moiety may be, inter alia, a cytotoxic factor. In particular embodiments, the chemical moiety is a polymer which increases the half-life of the antibody or fragment in the body of a subject. Suitable polymers include, but are not limited to, hydrophilic polymers. Such hydrophilic polymers may include but are not limited to polyethylene glycol (PEG) (e.g., PEG with a molecular weight of 2 kDa, 5 kDa, 10 kDa, 12 kDa, 20 kDa, 30 kDa or 40 kDa). Such hydrophilic polymers may include but are not limited to dextran. Such hydrophilic polymers may include but are not limited to monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545-553) disclose conjugating antibodies with PEG which is attached to a radiometal chelator (diethylenetriaminpentaacetic acid (DTPA)).

The antibodies and antigen-binding fragments thereof disclosed herein may also be conjugated with labels such as ⁹⁹Tc, ^{99m}Tc, ⁸⁶Y, ⁸⁸Y, ⁹⁰Y, ¹¹¹In, ³²P, ¹⁴C, ¹²³I, ¹²⁴I, ¹²⁵I, ³H, ¹³¹I, ¹¹C, ¹⁵O, ¹³N, ¹⁸F, ¹⁹F, ³⁵S, ⁵¹Cr, ⁵⁷To, ²²⁶Ra, ⁶⁰Co, ⁵⁹Fe, ⁵⁷Se, ¹⁵²Eu, ⁶¹Cu, ⁶²Cu, ⁶⁴Cu, ⁶⁷Cu, ⁶⁶Ga, ⁶⁷Ga, ⁶⁸Ga, ⁷²Ga, ⁴⁵Ti, ⁸⁹Zr, ²¹⁷Ci, ²¹¹At, ²¹²Pb, ¹⁷⁷Lu, ⁴⁴Sc, ⁴⁷Sc, ¹⁰⁹Pd, ²³⁴Th, and ⁴⁰K, ¹⁵⁷Gd, ⁵⁵Mn, ⁵²Tr, and ⁵⁶Fe.

The antibodies disclosed herein may also be PEGylated, for example to increase its biological (e.g., serum) half-life. The antigen-binding fragments disclosed herein may also be PEGylated. To PEGylate an antibody or fragment, the antibody or fragment, typically is reacted with a reactive form of polyethylene glycol (PEG), such as a reactive ester or aldehyde derivative of PEG, under conditions in which one or more PEG groups become attached to the antibody or antibody fragment. In particular embodiments, the PEGylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody or fragment to be PEGylated is an aglycosylated antibody or fragment. Methods for PEGylating proteins are known in the art and can be applied to the antibodies of the invention. See, e.g., EP 0 154 316 and EP 0 401 384.

The antibodies disclosed herein may also be conjugated with fluorescent labels. The antibodies disclosed herein may also be conjugated with chemilluminescent labels. The antigen-binding fragments disclosed herein may also be conjugated with fluorescent labels. The antigen-binding fragments disclosed herein may also be conjugated with chemilluminescent labels. This includes fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

The antibodies of the invention may also be conjugated to a cytotoxic agent. The antigen-binding fragments of the antibodies of the invention may also be conjugated to a cytotoxic agent. The antibodies and antigen-binding fragments thereof of the invention may also be conjugated to a cytotoxic agent such as auristatin F, paclitaxel, docetaxel, vincristine, CC-1065, SN-38, topotecan, morpholino doxorubicin, lysoxin, cyanomorpholino doxorubicin, Dolastatin-10, echinomycin, combretatstatin, chalicheamicin, maytansine, DM-1, netropsin, diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, Momordica charantia inhibitor, curcin, crotin, Saponaria officinalis inhibitor, mitogellin, restrictocin, phenomycin, and enomycin. This list is not meant to be limiting. The antibodies of the invention may be conjugated to auristatin F. The antibodies of the invention may be conjugated to paclitaxel. The antibodies of the invention may be conjugated to docetaxel. The antibodies of the invention may be conjugated to vincristine. The antibodies of the invention may be conjugated to CC-1065. The antibodies of the invention may be conjugated to SN-38. The antibodies of the invention may be conjugated to topotecan. The antibodies of the invention may be conjugated to morpholino doxorubicin. The antibodies of the invention may be conjugated to lysoxin. The antibodies of the invention may be conjugated to cyanomorpholino doxorubicin. The antibodies of the invention may be conjugated to Dolastatin-10. The antibodies of the invention may be conjugated to echinomycin. The antibodies of the invention may be conjugated to combretatstatin. The antibodies of the invention may be conjugated to chalicheamicin. The antibodies of the invention may be conjugated to maytansine. The antibodies of the invention may be conjugated to DM-1. The antibodies of the invention may be conjugated to netropsin. The antibodies of the invention may be conjugated to diptheria toxin. The antibodies of the invention may be conjugated to Pseudomonas aeruginosa exotoxin A chain. The antibodies of the invention may be conjugated to ricin A chain. The antibodies of the invention may be conjugated to abrin A chain. The antibodies of the invention may be conjugated to modeccin A chain. The antibodies of the invention may be conjugated to alpha-sarcin. The antibodies of the invention may be conjugated to Aleurites fordii proteins and compounds (e.g., fatty acids). The antibodies of the invention may be conjugated to dianthin proteins. The antibodies of the invention may be conjugated to Phytoiacca americana protein PAPI. The antibodies of the invention may be conjugated to Phytoiacca americana protein PAPII. The antibodies of the invention may be conjugated to Phytoiacca americana protein PAP-S. The antibodies of the invention may be conjugated to Momordica charantia inhibitor. The antibodies of the invention may be conjugated to curcin. The antibodies of the invention may be conjugated to crotin. The antibodies of the invention may be conjugated to Saponaria officinalis inhibitor. The antibodies of the invention may be conjugated to mitogellin. The antibodies of the invention may be conjugated to restrictocin. The antibodies of the invention may be conjugated to phenomycin. The antibodies of the invention may be conjugated to enomycin. The antigen-binding fragments of the antibodies of the invention may be conjugated to auristatin F. The antigen-binding fragments of the antibodies of the invention may be conjugated to paclitaxel. The antigen-binding fragments of the antibodies of the invention may be conjugated to docetaxel. The antigen-binding fragments of the antibodies of the invention may be conjugated to vincristine. The antigen-binding fragments of the antibodies of the invention may be conjugated to CC-1065. The antigen-binding fragments of the antibodies of the invention may be conjugated to SN-38. The antigen-binding fragments of the antibodies of the invention may be conjugated to topotecan. The antigen-binding fragments of the antibodies of the invention may be conjugated to morpholino doxorubicin. The antibodies of the invention may be conjugated to lysoxin. The antigen-binding fragments of the antibodies of the invention may be conjugated to cyanomorpholino doxorubicin. The antigen-binding fragments of the antibodies of the invention may be conjugated to Dolastatin-10. The antigen-binding fragments of the antibodies of the invention may be conjugated to echinomycin. The antigen-binding fragments of the antibodies of the invention may be conjugated to combretatstatin. The antigen-binding fragments of the antibodies of the invention may be conjugated to chalicheamicin. The antigen-binding fragments of the antibodies of the invention may be conjugated to maytansine. The antigen-binding fragments of the antibodies of the invention may be conjugated to DM-1. The antigen-binding fragments of the antibodies of the invention may be conjugated to netropsin. The antigen-binding fragments of the antibodies of the invention may be conjugated to diptheria toxin. The antigen-binding fragments of the antibodies of the invention may be conjugated to Pseudomonas aeruginosa exotoxin A chain. The antibodies of the invention may be conjugated to ricin A chain. The antigen-binding fragments of the antibodies of the invention may be conjugated to abrin A chain. The antigen-binding fragments of the antibodies of the invention may be conjugated to modeccin A chain. The antigen-binding fragments of the antibodies of the invention may be conjugated to alpha-sarcin. The antigen-binding fragments of the antibodies of the invention may be conjugated to Aleurites fordii proteins and compounds (e.g., fatty acids). The antigen-binding fragments of the antibodies of the invention may be conjugated to dianthin proteins. The antigen-binding fragments of the antibodies of the invention may be conjugated to Phytoiacca americana protein PAPI. The antigen-binding fragments of the antibodies of the invention may be conjugated to Phytoiacca americana protein PAPII. The antigen-binding fragments of the antibodies of the invention may be conjugated to Phytoiacca americana protein PAP-S. The antigen-binding fragments of the antibodies of the invention may be conjugated to Momordica charantia inhibitor. The antigen-binding fragments of the antibodies of the invention may be conjugated to curcin. The antigen-binding fragments of the antibodies of the invention may be conjugated to crotin. The antigen-binding fragments of the antibodies of the invention may be conjugated to Saponaria officinalis inhibitor. The antigen-binding fragments of the antibodies of the invention may be conjugated to mitogellin. The antigen-binding fragments of the antibodies of the invention may be conjugated to restrictocin. The antigen-binding fragments of the antibodies of the invention may be conjugated to phenomycin. The antigen-binding fragments of the antibodies of the invention may be conjugated to enomycin.

The antibodies of the invention may also be conjugated to an anticancer agent. The antibodies of the invention may also be conjugated to an anticancer agent such as such as erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum (II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo[4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl) phenoxy]-N,N-dimethylethanamine, NOLVADEX®, ISTUBAL®, VALODEX®), and doxorubicin (ADRIAMYCIN®). Additional commercially or clinically available anti-cancer agents comprise oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, W O 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, Ill.), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); vinorelbine (NAVELBINE®); capecitabine (XELODA®, Roche), tamoxifen (including NOLVADEX®; tamoxifen citrate, FARESTON® (toremifine citrate) MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca). This list is not meant to be limiting.

The antigen-binding fragments of the antibodies of the invention may also be conjugated to an anticancer agent. The antigen-binding fragments of the antibodies of the invention may also be conjugated to an anticancer agent such as such as erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210 Oct. 9, Pfizer), cisplatin (cis-diamine, dichloroplatinum (II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo[4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl) phenoxy]-N,N-dimethylethanamine, NOLVADEX®, ISTUBAL®, VALODEX®), and doxorubicin (ADRIAMYCIN®). Additional commercially or clinically available anti-cancer agents comprise oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, W O 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, Ill.), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); vinorelbine (NAVELBINE®); capecitabine (XELODA®, Roche), tamoxifen (including NOLVADEX®; tamoxifen citrate, FARESTON® (toremifine citrate) MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca). This list is not meant to be limiting.

The antibodies and antigen-binding fragments herein may be detectably labeled using paramagnetic chelates, microparticles, superparamagnetic particles; incorporated into ultrasound bubbles, microparticles, microspheres, emulsions, etc.

A metal chelator(s) is a molecule having one or more polar groups that act as a ligand for, and complex with, a paramagnetic metal. Suitable chelators are known in the art and include acids with methylene phosphonic acid groups, methylene carbohydroxamine acid groups, carboxyethylidene groups, or carboxymethylene groups. Examples of chelators include, but are not limited to, diethylenetriaminepentaacetic acid (DTPA), 1,4,7,10-tetraazacyclo-tetradecane-1,4,7,10-tetraacetic acid (DOTA), 1-substituted 1,4,7,-tricarboxymethyl-1,4,7,10-teraazacyclododecane (DO3A), ethylenediaminetetraacetic acid (EDTA), and 1,4,8,11-tetra-azacyclotetradecane-1,4,8,11-tetraacetic acid (TETA). Additional chelating ligands are ethylene bis-(2-hydroxy-phenylglycine) (EHPG), and derivatives thereof, including 5-C1-EHPG, 5Br-EHPG, 5-Me-EHPG, 5t-Bu-EHPG, and 5 sec-Bu-EHPG; benzodiethylenetriamine pentaacetic acid (benzo-DTPA) and derivatives thereof, including dibenzo-DTPA, phenyl-DTPA, diphenyl-DTPA, benzyl-DTPA, and dibenzyl DTPA; bis-2 (hydroxybenzyl)-ethylene-diaminediacetic acid (HBED) and derivatives thereof; the class of macrocyclic compounds, which contain at least 3 carbon atoms, more preferably at least 6, and at least two heteroatoms (O and/or N), which macrocyclic compounds can consist of one ring, or two or three rings joined together at the hetero ring elements, e.g., benzo-DOTA, dibenzo-DOTA, and benzo-NOTA, where NOTA is 1,4,7-triazacyclononane N,N′,N″-triacetic acid, benzo-TETA, benzo-DOTMA, where DOTMA is 1,4,7,10-tetraazacyclotetradecane-1,4,7,10-tetra(methyl tetraacetic acid), and benzo-TETMA, where TETMA is 1,4,8,11-tetraazacyclotetradecane-1,4,8,11-(methyl tetraacetic acid); derivatives of 1,3-propylene-diaminetetraacetic acid (PDTA) and triethylenetetraaminehexaacetic acid (TTHA); derivatives of 1,5,10-N,N′,N″-tris(2,3-dihydroxybenzoyl)-tricatecholate (LICAM); and 1,3,5-N,N′,N″-tris(2,3-dihydroxybenzoyl) aminomethylbenzene (MECAM). Examples of representative chelators and chelating groups contemplated by the present invention are described in WO 98/18496, WO 86/06605, WO 91/03200, WO 95/28179, WO 96/23526, WO 97/36619, PCT/US98/01473, PCT/US98/20182, and U.S. Pat. Nos. 4,899,755, 5,474,756, 5,846,519 and 6,143,274, all of which are hereby incorporated by reference.

Any method known in the art for conjugating the antibodies and antigen-binding fragments thereof of the invention to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem, and Cytochem. 30:407. Methods for conjugating antibodies and fragments are conventional and very well known in the art.

Chemical cross-linkers may be classified on the basis of the following:
1. Functional groups and chemical specificity;
2. length and composition of the cross-bridge;
3. whether the cross-linking groups are similar (homobifunctional) or different (heterobifunctional);
4. whether the groups react chemically or photochemically;
5. whether the reagent is cleavable; and
6. whether the reagent can be radiolabeled or tagged with another label.

Reactive groups on antibodies and labels that can be targeted using a cross-linker include primary amines, carbonyls, carbohydrates and carboxylic acids. In addition, many reactive groups can be coupled nonselectively using a cross-linker such as photoreactive phenyl azides. For suitable reagents, see Pierce 2003-2004 Applications Handbook and Catalog #1600926, which is hereby incorporated by reference.

Many factors must be considered to determine optimum cross-linker-to-target molar ratios. Depending on the application, the degree of conjugation is an important factor. For example, when preparing immunogen conjugates, a high degree of conjugation is normally desired to increase the immunogenicity of the antigen. However, when conjugating to an antibody or an enzyme, a low-to-moderate degree of conjugation may be optimal to ensure that the biological activity of the protein is retained. It is also important to consider the number of reactive groups on the surface of the protein. If there are numerous target groups, a lower cross-linker-to-protein ratio can be used. For a limited number of potential targets, a higher cross-linker-to-protein ratio may be required. This translates into more cross-linker per gram for a small molecular weight protein.

Conformational changes of proteins associated with a particular interaction may also be analyzed by performing cross-linking studies before and after the interaction. A comparison is made by using different arm-length cross-linkers and analyzing the success of conjugation. The use of cross-linkers with different reactive groups and/or spacer arms may be desirable when the conformation of the protein changes such that hindered amino acids become available for cross-linking.

Cross-linkers are available with varying lengths of spacer arms or bridges connecting the reactive ends. The most apparent attribute of the bridge is its ability to deal with steric considerations of the moieties to be linked. Because steric effects dictate the distance between potential reaction sites for cross-linking, different lengths of bridges may be considered for the interaction. Shorter spacer arms are often used in intramolecular cross-linking studies, while intermolecular cross-linking is favored with a cross-linker containing a longer spacer arm.

The inclusion of polymer portions (e.g., polyethylene glycol (“PEG”) homopolymers, polypropylene glycol homopolymers, other alkyl-polyethylene oxides, bis-polyethylene oxides and co-polymers or block co-polymers of poly(alkylene oxides)) in cross-linkers can, under certain circumstances be advantageous. See, e.g., U.S. Pat. Nos. 5,643,575, 5,672,662, 5,705,153, 5,730,990, 5,902,588, and 5,932,462; and Topchieva et al., Bioconjug. Chem. 6:380-8, 1995). For example, U.S. Pat. No. 5,672,662 discloses bifunctional cross-linkers comprising a PEG polymer portion and a single ester linkage. Such molecules are said to provide a half-life of about 10 to 25 minutes in water.

Designing a cross-linker involves selection of the functional moieties to be employed. The choice of functional moieties is entirely dependent upon the target sites available on the species to be crosslinked. Some species (e.g., proteins) may present a number of available sites for targeting (e.g., lysine ¿-amino groups, cysteine sulfhydryl groups, glutamic acid carboxyl groups, etc.), and selection of a particular functional moiety may be made empirically in order to best preserve a biological property of interest (e.g., binding affinity of an antibody, catalytic activity of an enzyme, etc.)

### Coupling Through Amine Groups

Imidoester and N-hydroxysuccinimidyl (“NHS”) esters are typically employed as amine-specific functional moieties. NHS esters yield stable products upon reaction with primary or secondary amines. Coupling is efficient at physiological pH, and NHS-ester cross-linkers are more stable in solution than their imidate counterparts. Homobifunctional NHS-ester conjugations are commonly used to cross-link amine-containing proteins in either one-step or two-step reactions. Primary amines are the principle targets for NHS-esters. Accessible α-amine groups present on the N-termini of proteins react with NHS-esters to form amides. However, because α-amines on a protein are not always available, the reaction with side chains of amino acids become important. While five amino acids have nitrogen in their side chains, only the s-amino group of lysine reacts significantly with NHS-esters. A covalent amide bond is formed when the NHS-ester cross-linking agent reacts with primary amines, releasing N-hydroxysuccinimide.

### Coupling Through Sulfhydryl Groups

Maleimides, alkyl and aryl halides, α-haloacyls, and pyridyl disulfides are typically employed as sulfhydryl-specific functional moieties. The maleimide group is specific for sulfhydryl groups when the pH of the reaction mixture is kept between pH 6.5 and 7.5. At pH 7, the reaction of the maleimides with sulfhydryls is 1000-fold faster than with amines. Maleimides do not react with tyrosines, histidines or methionines. When free sulfhydryls are not present in sufficient quantities, they can often be generated by reduction of available disulfide bonds.

### Coupling Through Carboxyl Groups

Carbodiimides couple carboxyls to primary amines or hydrazides, resulting in formation of amide or hydrazone bonds. Carbodiimides are unlike other conjugation reactions in that no cross-bridge is formed between the carbodiimide and the molecules being coupled; rather, a peptide bond is formed between an available carboxyl group and an available amine group. Carboxy termini of proteins can be targeted, as well as glutamic and aspartic acid side chains. In the presence of excess cross-linker, polymerization may occur because proteins contain both carboxyls and amines. No cross-bridge is formed, and the amide bond is the same as a peptide bond, so reversal of the cross-linking is impossible without destruction of the protein.

### Nonselective Labeling

A photoaffinity reagent is a compound that is chemically inert but becomes reactive when exposed to ultraviolet or visible light. Arylazides are photoaffinity reagents that are photolyzed at wavelengths between 250-460 nm, forming a reactive aryl nitrene. The aryl nitrene reacts nonselectively to form a covalent bond. Reducing agents must be used with caution because they can reduce the azido group.

### Carbonyl Specific Cross-Linkers

Carbonyls (aldehydes and ketones) react with amines and hydrazides at pH 5-7. The reaction with hydrazides is faster than with amines, making this useful for site-specific cross-linking. Carbonyls do not readily exist in proteins; however, mild oxidation of sugar moieties using sodium metaperiodate will convert vicinal hydroxyls to aldehydes or ketones.

### Experimental and Diagnostic Uses

The anti-CD103 antibodies disclosed herein may be used as affinity purification agents. The anti-CD103 antigen-binding fragments disclosed herein may be used as affinity purification agents. In this process, the anti-CD103 antibodies and antigen-binding fragments thereof are immobilized on a solid phase such a Sephadex, glass or agarose resin or filter paper, using methods well known in the art. The immobilized antibody or fragment is contacted with a sample containing the CD103 protein (or a fragment thereof) to be purified, and thereafter the support is washed with a suitable solvent that will remove substantially all the material in the sample except the CD103 protein, which is bound to the immobilized antibody or fragment. Finally, the support is washed with a solvent which elutes the bound CD103 (e.g., protein A). Such immobilized antibodies and fragments form part of the present invention.

Further provided are antigens for generating secondary antibodies which are useful for example for performing Western blots and other immunoassays discussed herein.

anti-CD103 antibodies (e.g., humanized antibodies) and antigen-binding fragments thereof may also be useful in diagnostic assays for CD103 protein, e.g., detecting its expression in specific cells, tissues, or serum, e.g., myeloid cells such as monocytes, macrophages, neutrophils, basophils, eosinophils, and dendritic cells. Such diagnostic methods may be useful in various disease diagnoses.

The present invention includes ELISA assays (enzyme-linked immunosorbent assay) incorporating the use of an anti-CD103 antibody or antigen-binding fragment thereof disclosed herein.

For example, such a method comprises the following steps:
(a) coat a substrate (e.g., surface of a microtiter plate well, e.g., a plastic plate) with anti-CD103 antibody or antigen-binding fragment thereof;
(b) apply a sample to be tested for the presence of CD103 to the substrate;
(c) wash the plate, so that unbound material in the sample is removed;
(d) apply detectably labeled antibodies (e.g., enzyme-linked antibodies) which are also specific to the CD103 antigen;
(e) wash the substrate, so that the unbound, labeled antibodies are removed;
(f) if the labeled antibodies are enzyme linked, apply a chemical which is converted by the enzyme into a fluorescent signal; and
(g) detect the presence of the labeled antibody.

Detection of the label associated with the substrate indicates the presence of the CD103 protein.

In a further embodiment, the labeled antibody or antigen-binding fragment thereof is labeled with peroxidase which react with ABTS (e.g., 2,2′-azino-bis(3-ethylbenzthiazoline-6-sulphonic acid)) or 3,3′,5,5′-Tetramethylbenzidine to produce a color change which is detectable. Alternatively, the labeled antibody or fragment is labeled with a detectable radioisotope (e.g., ³H) which can be detected by scintillation counter in the presence of a scintillant.

An anti-CD103 antibody or antigen-binding fragment thereof of the invention may be used in a Western blot or immune-protein blot procedure. Such a procedure forms part of the present invention and includes e.g.:
(1) optionally transferring proteins from a sample to be tested for the presence of CD103 (e.g., from a PAGE or SDS-PAGE electrophoretic separation of the proteins in the sample) onto a membrane or other solid substrate using a method known in the art (e.g., semi-dry blotting or tank blotting); contacting the membrane or other solid substrate to be tested for the presence of bound CD103 or a fragment thereof with an anti-CD103 antibody or antigen-binding fragment thereof of the invention.
(2) washing the membrane one or more times to remove unbound anti-CD103 antibody or fragment and other unbound substances; and
(3) detecting the bound anti-CD103 antibody or fragment.

Such a membrane may take the form of a nitrocellulose or vinyl-based (e.g., polyvinylidene fluoride (PVDF)) membrane to which the proteins to be tested for the presence of CD103 in a non-denaturing PAGE (polyacrylamide gel electrophoresis) gel or SDS-PAGE (sodium dodecyl sulfate polyacrylamide gel electrophoresis) gel have been transferred (e.g., following electrophoretic separation in the gel). Before contacting the membrane with the anti-CD103 antibody or fragment, the membrane is optionally blocked, e.g., with non-fat dry milk or the like so as to bind non-specific protein binding sites on the membrane.

Detection of the bound antibody or fragment indicates that the CD103 protein is present on the membrane or substrate and in the sample. Detection of the bound antibody or fragment may be by binding the antibody or fragment with a secondary antibody (an anti-immunoglobulin antibody) which is detectably labeled and, then, detecting the presence of the secondary antibody.

The anti-CD103 antibodies and antigen-binding fragments thereof disclosed herein may also be used for immunohistochemistry. Such a method forms part of the present invention and comprises, e.g.,
(1) contacting a cell (e.g., a sample containing myeloid cells such as monocytes, macrophages, neutrophils, basophils, eosinophils, and dendritic cells) to be tested for the presence of CD103 protein with an anti-CD103 antibody or antigen-binding fragment thereof of the invention; and
(2) detecting the antibody or fragment on or in the cell.

If the antibody or fragment itself is detectably labeled, it can be detected directly. Alternatively, the antibody or fragment may be bound by a detectably labeled secondary antibody which is detected.

Certain anti-CD103 antibodies and antigen-binding fragments thereof disclosed herein may also be used for in vivo tumor imaging. Such a method may include injection of a radiolabeled anti-CD103 antibody or antigen-binding fragment thereof into the body of a patient to be tested for the presence of a tumor associated with CD103 expression (e.g., which expresses CD103, for example, on the tumor cell surface) followed by nuclear imaging of the body of the patient to detect the presence of the labeled antibody or fragment e.g., at loci comprising a high concentration of the antibody or fragment which are bound to the tumor. The detection of the loci indicates the presence of the CD103⁺ tumor and tumor cells.

Imaging techniques include SPECT imaging (single photon emission computed tomography) or PET imaging (positron emission tomography). Labels include e.g., iodine-123 (¹²³I) and technetium-99m (^{99m}Tc), e.g., in conjunction with SPECT imaging or ¹¹C, ¹³N, ¹⁵O or ¹⁸F, e.g., in conjunction with PET imaging or Indium-111 (See e.g., Gordon et al., (2005) International Rev. Neurobiol. 67:385-440).

### Pharmaceutical Compositions and Administration and Therapeutic Uses

Antibodies of the present invention can inhibit CD103 signaling and, accordingly, in one aspect of the invention, certain antibodies disclosed herein are candidates for treating, or preventing certain conditions and diseases. The present invention provides methods for treating conditions and diseases wherein the course of the condition or disease can be influenced by CD103 signaling. The method includes administering to a subject in need of such treatment, a therapeutically effective amount of an antibody of the present invention.

Integrin family heterodimers play diverse and redundant roles in T-cell activation, homing, and delivery of effector function. The CD103 integrin heterodimer was initially identified by its expression on T cells in the vertebrate gut mucosa, where it is expressed at high levels by >95% of intestinal intraepithelial lymphocytes (iIEL) and ^{~}40% lamina propria lymphocytes. CD103 recognizes the epithelial cell-specific ligand, E-cadherin. In normal mice and humans, CD8+ T cells that reside within the gut epithelium express high levels of CD103, and CD103 is widely expressed in intraepithelial lymphocytes, tumor infiltrating lymphocytes and certain dendritic cells. Previous studies have demonstrated that CD103 serves an important role in the cell lysis caused by tumor-specific infiltrating lymphocytes via interacting with its ligand, E-cadherin, on the tumor cells, triggering lytic granule polarization and exocytosis. Furthermore, the ligation of CD103 and E-cadherin promotes the adhesion of T cells to tumor cells and induces co-stimulation in activated cytotoxic T cells. These findings suggest that CD103 may be a target for enhancing tumor immunity.

In various embodiments, antibodies of the present invention block binding to E-cadherin; are used to deplete CD103+ cells; deplete CD103+ CD8+ effector cells; and/or are used to deplete tissue-resident memory T cells (TRM).

The present invention also provides a method of treating a CD103 signaling-mediated condition, comprising administering to a patient in need thereof an effective amount of one or more antibodies of the present invention. In some embodiments, CD103 signaling-mediated condition is an autoimmune, inflammatory, or neurodegenerative condition or cancer (see Rayburn, E. R. et al., Mol Cell Pharmacol.2009; 1 (1): 29-43 and Urbanska, A. M. et al., Cell Biochem Biophys.2015 July; 72 (3): 757-69).

### CD103 in Allograft Rejection

FACS analyses of transplant nephrectomy specimens revealed that a major subset of CD8 effectors that infiltrated allografts undergoing rejection episodes expressed high levels of CD103. Interestingly, CD103+ CD8+ effectors are most abundant in renal allografts undergoing rejection episodes in the context of chronic allograft nephropathy. Importantly, CD103+ CD8+ effectors are not present in peripheral lymphoid compartments (i.e., peripheral blood lymphocytes), and thus are not detectable by conventional immune monitoring approaches. However, CD103 mRNA is expressed by cells isolated from the urine of renal allograft recipients concomitant with clinical rejection, consistent with the intratubular localization of CD103+ CD8+ effectors during rejection episodes. The clinical observations noted above are consistent with a key role for CD103 in promoting destruction of graft epithelial compartments by CD8 effector populations, and support the hypothesis that CD103 expression is required for CD8-mediated destruction of graft epithelial elements. Antibodies of the invention as described herein can be used in the treatment of allograft rejection. Antibodies of the invention as described herein can be used in the prevention of allograft rejection. Antigen binding fragments of the invention as described herein can be used in the treatment of allograft rejection. Antigen binding fragments of the invention as described herein can be used in the prevention of allograft rejection.

### Tissue-Resident Memory T Cells

It has long been known that the recurrence of cutaneous chronic inflammation, especially psoriasis and FDE, frequently occurs in previously affected sites. Therefore, immunological memory has been proposed to be involved in flare-up reactivity and the chronicity of inflammatory disorders. With respect to the striking characteristics of T_{RM} cells (long-term survival and low migration in peripheral tissues), it has been suggested that skin T_{RM} cells may actively participate in the recurrence of inflammatory skin disorders.

The primary onset of psoriatic lesions is often followed by recurrence in previously resolved sites, and local resident memory T cells have been suggested to play a role in its development and flare-ups. CD8⁺ T cells in psoriatic lesions are highly activated and express large amounts of CD69 and CD103. In contrast, few T cells constitutively express these proteins in the peripheral blood, Furthermore, it is clear that T_{EM} cells interact with the vascular addressin E-selectin and are trafficked to the skin during infection or attack. More importantly, recent studies have shown that TCRαβ⁺ resident T cells accumulate in psoriatic resolved sites, even in normal-appearing skin, and that they are capable of producing IL-17 and IFN-γ to trigger psoriasiform responses. These findings support the important role of lesion-resident T cells in psoriasis development. Antibodies of the invention as described herein can be used in the treatment of psoriasis. Antibodies of the invention as described herein can be used in the prevention of psoriasis. Antigen binding fragments of the invention as described herein can be used in the treatment of psoriasis. Antigen binding fragments of the invention as described herein can be used in the prevention of psoriasis.

### Inflammatory Bowel Disease and CD103

An important pathological process increasingly recognised as driving intestinal inflammation and autoimmunity is the loss of immune homeostasis secondary to qualitative or quantitative defects in the regulatory T-cell (Treg) pool. Tregs can be broadly divided into two groups, thymic Tregs (tTregs) or peripherally induced Tregs (pTregs), based on their developmental origin. T cells could be converted into Foxp3-expressing CD4+CD25+Tregs by T-cell receptor (TCR) costimulation in the presence of transforming growth factor β (TGF-β).14 pTreg conversion in gut-associated lymphoid tissues (GALTs) was enhanced when naive CD4+ T cells encountered antigen in the presence of TGF-β, IL-2 and retinoic acid (RA). 15 16 This is facilitated by CD103+ DCs conditioned by the intestinal microenvironment to produce or activate TGF-β and provide RA.17 18 In the absence of CD103 expression, DCs fail to induce Treg development and produce proinflammatory cytokines. In patients with UC, it has been reported reported that CD103 expression on colonic CD4⁺ T cells was associated with increased production of proinflammatory Th1, Th17, and Th1/Th17 cytokines, and CD103⁺ DCs in patients with UC had an ability to drive Th1/Th2/Th17 cell responses. Therefore, the efficacy of targeting integrins might be explained by elimination of colitogenic CD103⁺ dendritic cells and blockade of lymphocytes recruitment. Antibodies of the invention as described herein can be used in the treatment of inflammatory bowel disease. Antibodies of the invention as described herein can be used in the prevention of inflammatory bowel disease. Antigen binding fragments of the invention as described herein can be used in the treatment of inflammatory bowel disease. Antigen binding fragments of the invention as described herein can be used in the prevention of inflammatory bowel disease.

### CD103+ Lymphoproliferative Disorders

Flow cytometric immunophenotyping is vital in the diagnosis of B-cell lymphoproliferative disorders (BC-LPDs), including B-cell chronic lymphocytic leukemia (CLL), mantle cell lymphoma (MCL), and hairy cell leukemia (HCL). In the diagnostic evaluation of low-grade B-cell lymphoproliferative processes, demonstration of CD103 positivity is indicative of a diagnosis of hairy cell leukemia (HCL) or its variant form HCLv.

In addition to these B-cell disorders, CD103 positivity is also a feature in a subset of T cell neoplasms. In one study of 184 cases representing most entities within the current World Health Organization classification of T cell neoplasms, 46% of gastrointestinal lymphomas, 40% of adult T cell leukaemia/lymphoma, and 6.9% of other neoplasms exhibited CD103 positivity. Likewise, Blastic Plasmacytoid Dendritic Cell Neoplasms (BPDCN) express CD103.

Antibodies of the invention as described herein can be used in the treatment of lymphoproliferative disorders. Antigen binding fragments of the invention as described herein can be used in the treatment of lymphoproliferative disorders. Lymphoproliferative disorders expressing CD103 that may be treated using the antibodies of the present invention include, but are not limited to, Hairy Cell leukemia, HCLv, intestinal and extraintestinal lymphomas, enteropathy-associated T-cell lymphoma (EATL), T-lymphoblastic leukemia/lymphoma (T-ALL), T-cell prolymphocytic leukemia (T-PLL), adult T cell leukemia/lymphoma (ATLL), mycosis fungoides (MF), anaplastic large cell lymphoma ALCL, cutaneous T-cell lymphoma (CTCL), and Blastic Plasmacytoid Dendritic Cell Neoplasm.

### CD103 in Tumorigenesis

It has been reported that tumor-associated CD103+ CD8 T cells have a tolerogenic phenotype with increased expression of CTLA-4 and IL-10 and decreased expression of IFN-γ, TNF-α, and granzymes. Moreover, CD103 has been described as a marker of CD4+ regulatory cells and is present on tolerogenic DCs. Direct targeting of CD103 by an anti-CD103 antibody that reduces CD103+ CD8 T cells in mice reportedly provides a therapeutic effect in the B16 melanoma and MC38 CRC models. Antibodies of the invention as described herein can be used in the treatment of tumorigenesis. Antibodies of the invention as described herein can be used in the prevention of tumorigenesis. Antigen binding fragments of the invention as described herein can be used in the treatment of tumorigenesis. Antigen binding fragments of the invention as described herein can be used in the prevention of tumorigenesis. Antibodies of the invention as described herein can be used in mitigating the progression of tumorigenesis.

### Therapeutic Applications of CD103 Antibodies of the Present Invention

The present invention provides the use of one or more antibodies of the present invention for inhibiting CD103 signaling in a cell.

The present invention provides the use of one or more antibodies of the present invention for inhibiting CD103 binding to E-cadherin and to E-cadherin-expressing cells.

The present invention further provides the use of one or more antibodies of the present invention for the treatment of a CD103-mediated condition.

The present invention provides the use of one or more antibodies of the present invention for depleting CD103-expressing cells.

The present invention further provides the use of one or more antibodies of the present invention in the manufacture of a medicament for one of the foregoing uses.

Antibodies of the invention as described herein, can be useful in treating a variety of diseases, where the modulation of CD103 signaling can provide therapeutic benefit. In some aspects, a compound of the invention inhibits CD103 signaling, and can be useful in treating a disease selected from the group consisting of atopic dermatitis, allergy, asthma, systemic inflammatory response syndrome (SIRS), sepsis, septic shock, atherosclerosis, celiac disease, dermatomyositis, scleroderma, interstitial cystitis, transplant rejection, graft-versus-host disease, Aicardi-Goutieres Syndrome, Hutchison Guilford progeria syndrome, Singleton-Merten Syndrome, proteasome-associated autoinflammatory syndrome, SAVI (STING-associated vasculopathy with onset in infancy), CANDLE (Chronic Atypical Neutrophilic Dermatosis with Lipodystrophy and Elevated Temperature) syndrome, chilblain lupus erythematosus, systemic lupus erythematosus, rheumatoid arthritis, juvenile rheumatoid arthritis, Wegener's disease, inflammatory bowel disease (e.g. ulcerative colitis, Crohn's disease), idiopathic thrombocytopenia purpura, thrombotic thrombocytopeni purpura, autoimmune thrombocytopenia, multiple sclerosis, psoriasis, IgA nephropathy, IgM polyneuropathies, glomerulonephritis, autoimmune myocarditis, myasthenia gravis, vasculitis, Type 1 diabetes, Type 2 diabetes, Sjorgen's syndrome, X-linked reticulate pigmentary disorder, polymyositis, spondyloenchondrodysplasia, age-related macular degeneration, Alzheimer's disease and Parkinson's disease. In some embodiments, compounds of the invention are useful in treating Aicardi-Goutieres Syndrome, X-linked reticulate pigmentary disorder, dermatomyositis, systemic lupus erythematosus, rheumatoid arthritis, multiple sclerosis, or Type I or Type II diabetes. Antibodies of the invention as described herein are preferably used in the treatment of inflammatory bowel disease. Antibodies of the invention as described herein are preferably used in the treatment of psoriasis.

The present invention provides a method of treating an autoimmune disease in a subject, comprising administering to the subject in need thereof a therapeutically effective amount of one or more antibodies of the present invention. In some embodiments, the autoimmune disease can be a type I interferonopathy (e.g., Aicardi-Goutieres Syndrome, Sjögren's syndrome, Singleton-Merten Syndrome, proteasome-associated autoinflammatory syndrome, SAVI (STING-associated vasculopathy with onset in infancy), CANDLE syndrome, chilblain lupus erythematosus, systemic lupus erythematosus, spondyloenchondrodysplasia), rheumatoid arthritis, juvenile rheumatoid arthritis, idiopathic thrombocytopenia purpura, autoimmune myocarditis, thrombotic thrombocytopenia purpura, autoimmune thrombocytopenia, psoriasis, Type 1 diabetes, or Type 2 diabetes.

The present invention provides a method of treating an inflammatory disease in a subject, comprising administering to the subject in need thereof a therapeutically effective amount of one or more antibodies of the present invention. For example, the inflammatory disease can be selected from the group consisting of atopic dermatitis, allergy, asthma, systemic inflammatory response syndrome (SIRS), sepsis, septic shock, atherosclerosis, celiac disease, dermatomyositis, scleroderma, interstitial cystitis, transplant rejection, graft-versus-host disease, Aicardi-Goutieres Syndrome, Hutchison Guilford progeria syndrome, Singleton-Merten Syndrome, proteasome-associated autoinflammatory syndrome, SAVI (STING-associated vasculopathy with onset in infancy), CANDLE (Chronic Atypical Neutrophilic Dermatosis with Lipodystrophy and Elevated Temperature) syndrome, chilblain lupus erythematosus, systemic lupus erythematosus, rheumatoid arthritis, juvenile rheumatoid arthritis, Wegener's disease, inflammatory bowel disease (e.g. ulcerative colitis, Crohn's disease), idiopathic thrombocytopenia purpura, thrombotic thrombocytopeni purpura, autoimmune thrombocytopenia, multiple sclerosis, psoriasis, IgA nephropathy, IgM polyneuropathies, glomerulonephritis, autoimmune myocarditis, myasthenia gravis, vasculitis, Type 1 diabetes, Type 2 diabetes, Sjorgen's syndrome, X-linked reticulate pigmentary disorder, polymyositis, spondyloenchondrodysplasia, age-related macular degeneration, Alzheimer's disease and Parkinson's disease. In some embodiments, compounds of the invention are useful in treating Aicardi-Goutieres Syndrome, X-linked reticulate pigmentary disorder, dermatomyositis, systemic lupus erythematosus, rheumatoid arthritis, multiple sclerosis, or Type I or Type II diabetes.

The present invention further provides a method of treating neurodegenerative diseases in a subject, comprising administering to the subject in need thereof a therapeutically effective amount of one or more antibodies of the present invention. For example, the neurodegenerative disease can be Alzheimer's disease, Parkinson's disease, multiple sclerosis, IgM polyneuropathies, or myasthenia gravis.

The present invention further provides a method of treating malignancies expressing CD103. For example, the malignancies may be T- and B-cell lymphomas, and particularly Hairy Cell leukemia, HCLv, intestinal and extraintestinal lymphomas, enteropathy-associated T-cell lymphoma (EATL), T-lymphoblastic leukemia/lymphoma (T-ALL), T-cell prolymphocytic leukemia (T-PLL), adult T cell leukemia/lymphoma (ATLL), mycosis fungoides (MF), anaplastic large cell lymphoma ALCL, cutaneous T-cell lymphoma (CTCL), Sezary Syndrome (SS).

To prepare pharmaceutical or sterile compositions of the anti-CD103 antibodies and antigen-binding fragments of the invention, the antibody or antigen-binding fragment thereof is admixed with a pharmaceutically acceptable carrier or excipient. See, e.g., Remington's Pharmaceutical Sciences and U.S. Pharmacopeia: National Formulary, Mack Publishing Company, Easton, PA (1984).

Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY).

Toxicity and therapeutic efficacy of the antibodies of the invention, administered alone or in combination with another therapeutic agent, can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index (LD50/ED50). The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration.

In a further embodiment, a further therapeutic agent that is administered to a subject in association with an anti-CD103 antibody or antigen-binding fragment thereof of the invention in accordance with the Physicians' Desk Reference 2003 (Thomson Healthcare; 57th edition (Nov. 1, 2002)).

The mode of administration can vary. Routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

In particular embodiments, the anti-CD103 antibodies or antigen-binding fragments thereof of the invention can be administered by an invasive route such as by injection. In further embodiments of the invention, an anti-CD103 antibody or antigen-binding fragment thereof, or pharmaceutical composition thereof, is administered intravenously, subcutaneously, intramuscularly, intraarterially, intratumorally, or by inhalation, aerosol delivery. Administration by non-invasive routes (e.g., orally; for example, in a pill, capsule or tablet) is also within the scope of the present invention.

The present invention provides a vessel (e.g., a plastic or glass vial, e.g., with a cap or a chromatography column, hollow bore needle or a syringe cylinder) comprising any of the antibodies or antigen-binding fragments of the invention or a pharmaceutical composition thereof. The present invention also provides an injection device comprising any of the antibodies or antigen-binding fragments of the invention or a pharmaceutical composition thereof. An injection device is a device that introduces a substance into the body of a patient via a parenteral route, e.g., intramuscular, subcutaneous or intravenous. For example, an injection device may be a syringe (e.g., pre-filled with the pharmaceutical composition, such as an auto-injector) which, for example, includes a cylinder or barrel for holding fluid to be injected (e.g., antibody or fragment or a pharmaceutical composition thereof), a needle for piecing skin and/or blood vessels for injection of the fluid; and a plunger for pushing the fluid out of the cylinder and through the needle bore. In an embodiment of the invention, an injection device that comprises an antibody or antigen-binding fragment thereof of the present invention or a pharmaceutical composition thereof is an intravenous (IV) injection device. Such a device includes the antibody or fragment or a pharmaceutical composition thereof in a cannula or trocar/needle which may be attached to a tube which may be attached to a bag or reservoir for holding fluid (e.g., saline; or lactated ringer solution comprising NaCl, sodium lactate, KCl, CaCl₂) and optionally including glucose) introduced into the body of the patient through the cannula or trocar/needle. The antibody or fragment or a pharmaceutical composition thereof may, in an embodiment of the invention, be introduced into the device once the trocar and cannula are inserted into the vein of a subject and the trocar is removed from the inserted cannula. The IV device may, for example, be inserted into a peripheral vein (e.g., in the hand or arm); the superior vena cava or inferior vena cava, or within the right atrium of the heart (e.g., a central IV); or into a subclavian, internal jugular, or a femoral vein and, for example, advanced toward the heart until it reaches the superior vena cava or right atrium (e.g., a central venous line). In an embodiment of the invention, an injection device is an autoinjector; a jet injector or an external infusion pump. A jet injector uses a high-pressure narrow jet of liquid which penetrate the epidermis to introduce the antibody or fragment or a pharmaceutical composition thereof to a patient's body. External infusion pumps are medical devices that deliver the antibody or fragment or a pharmaceutical composition thereof into a patient's body in controlled amounts. External infusion pumps may be powered electrically or mechanically. Different pumps operate in different ways, for example, a syringe pump holds fluid in the reservoir of a syringe, and a moveable piston controls fluid delivery, an elastomeric pump holds fluid in a stretchable balloon reservoir, and pressure from the elastic walls of the balloon drives fluid delivery. In a peristaltic pump, a set of rollers pinches down on a length of flexible tubing, pushing fluid forward. In a multi-channel pump, fluids can be delivered from multiple reservoirs at multiple rates.

The pharmaceutical compositions disclosed herein may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556. Such needleless devices comprising the pharmaceutical composition are also part of the present invention. The pharmaceutical compositions disclosed herein may also be administered by infusion. Examples of well-known implants and modules for administering the pharmaceutical compositions include those disclosed in: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art and those comprising the pharmaceutical compositions of the present invention are within the scope of the present invention.

Alternately, one may administer the anti-CD103 antibody or antigen-binding fragment of the invention in a local rather than systemic manner, for example, via injection of the antibody or fragment directly into a tumor. Furthermore, one may administer the antibody or fragment in a targeted drug delivery system, for example, in a liposome coated with a tissue-specific antibody, targeting, for example, a tumor. The liposomes will be targeted to and taken up selectively by the afflicted tissue. Such methods and liposomes are part of the present invention.

The administration regimen depends on several factors, including the serum or tissue turnover rate of the therapeutic antibody or antigen-binding fragment, the level of symptoms, the immunogenicity of the therapeutic antibody, and the accessibility of the target cells in the biological matrix. Preferably, the administration regimen delivers sufficient therapeutic antibody or fragment to effect improvement in the target disease state, while simultaneously minimizing undesired side effects. Accordingly, the amount of biologic delivered depends in part on the particular therapeutic antibody and the severity of the condition being treated. Guidance in selecting appropriate doses of therapeutic antibodies or fragments is available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert, et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602).

Determination of the appropriate dose is made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent. In the case of human subjects, for example, humanized and fully human antibodies may be desirable.

Antibodies or antigen-binding fragments thereof disclosed herein may be provided by continuous infusion, or by doses administered, e.g., daily, 1-7 times per week, weekly, bi-weekly, monthly, bimonthly, quarterly, semiannually, annually etc. Doses may be provided, e.g., intravenously, subcutaneously, topically, orally, nasally, rectally, intramuscular, intracerebrally, intraspinally, or by inhalation. A total weekly dose is generally at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 5.0 mg/mL, 10 mg/kg, 25 mg/kg, 50 mg/kg or more (see, e.g., Yang, et al. (2003) New Engl. J. Med. 349:427-434; Herold, et al. (2002) New Engl. J. Med. 346:1692-1698; Liu, et al. (1999) J. Neurol. Neurosurg. Psych. 67:451-456; Portielji, et al. (20003) Cancer Immunol. Immunother. 52:151-144). Doses may also be provided to achieve a pre-determined target concentration of anti-CD103 antibody in the subject's serum, such as 0.1, 0.3, 1, 3, 10, 30, 100, 300 μg/mL or more. In other embodiments, An anti-CD103 antibody of the present invention is administered, e.g., subcutaneously or intravenously, on a weekly, biweekly, “every 4 weeks,” monthly, bimonthly, or quarterly basis at 10, 20, 50, 80, 100, 200, 500, 1000 or 2500 mg/subject.

As used herein, the term “effective amount” refer to an amount of an anti-CD103 or antigen-binding fragment thereof of the invention that, when administered alone or in combination with an additional therapeutic agent to a cell, tissue, or subject, is effective to cause a measurable improvement in one or more symptoms of disease, for example cancer or the progression of cancer. An effective dose further refers to that amount of the antibody or fragment sufficient to result in at least partial amelioration of symptoms, e.g., tumor shrinkage or elimination, lack of tumor growth, increased survival time. When applied to an individual active ingredient administered alone, an effective dose refers to that ingredient alone. When applied to a combination, an effective dose refers to combined amounts of the active ingredients that result in the therapeutic effect, whether administered in combination, serially or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity.

### Kits

Further provided are kits comprising one or more components that include, but are not limited to, an anti-CD103 antibody or antigen-binding fragment, as discussed herein in association with one or more additional components including, but not limited to a pharmaceutically acceptable carrier and/or a therapeutic agent, as discussed herein. The antibody or fragment and/or the therapeutic agent can be formulated as a pure composition or in combination with a pharmaceutically acceptable carrier, in a pharmaceutical composition.

In one embodiment, the kit includes an anti-CD103 antibody or antigen-binding fragment thereof of the invention or a pharmaceutical composition thereof in one container (e.g., in a sterile glass or plastic vial) and/or a therapeutic agent and a pharmaceutical composition thereof in another container (e.g., in a sterile glass or plastic vial).

In another embodiment, the kit comprises a combination of the invention, including an anti-CD103 antibody or antigen-binding fragment thereof of the invention along with a pharmaceutically acceptable carrier, optionally in combination with one or more therapeutic agents formulated together, optionally, in a pharmaceutical composition, in a single, common container.

If the kit includes a pharmaceutical composition for parenteral administration to a subject, the kit can include a device for performing such administration. For example, the kit can include one or more hypodermic needles or other injection devices as discussed above.

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

### Detection Kits

Also provided are diagnostic or detection reagents and kits comprising one or more such reagents for use in a variety of detection assays, including for example, immunoassays such as ELISA (sandwich-type or competitive format). The kit's components may be pre-attached to a solid support, or may be applied to the surface of a solid support when the kit is used. In some embodiments of the invention, the signal generating means may come pre-associated with an antibody or fragment of the invention or may require combination with one or more components, e.g., buffers, antibody-enzyme conjugates, enzyme substrates, or the like, prior to use. Kits may also include additional reagents, e.g., blocking reagents for reducing nonspecific binding to the solid phase surface, washing reagents, enzyme substrates, and the like. The solid phase surface may be in the form of a tube, a bead, a microtiter plate, a microsphere, or other materials suitable for immobilizing proteins, peptides, or polypeptides. In particular aspects, an enzyme that catalyzes the formation of a chemilluminescent or chromogenic product or the reduction of a chemilluminescent or chromogenic substrate is a component of the signal generating means. Such enzymes are well known in the art. Kits may comprise any of the capture agents and detection reagents described herein. Optionally the kit may also comprise instructions for carrying out the methods of the invention.

The detection kits disclosed herein may also be prepared that comprise at least one of the antibody, peptide, antigen-binding fragment, or polynucleotide disclosed herein and instructions for using the composition as a detection reagent. Containers for use in such kits may typically comprise at least one vial, test tube, flask, bottle, syringe or other suitable container, into which one or more of the detection composition(s) may be placed, and preferably suitably aliquoted. The kits disclosed herein will also typically include a means for containing the vial(s) in close confinement for commercial sale, such as, e.g., injection or blow-molded plastic containers into which the desired vial(s) are retained. Where a radiolabel, chromogenic, fluorigenic, or other type of detectable label or detecting means is included within the kit, the labeling agent may be provided either in the same container as the detection or therapeutic composition itself, or may alternatively be placed in a second distinct container means into which this second composition may be placed and suitably aliquoted. Alternatively, the detection reagent and the label may be prepared in a single container means, and in most cases, the kit will also typically include a means for containing the vial(s) in close confinement for commercial sale and/or convenient packaging and delivery.

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, NY; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, NY, pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, New York; Kay et al. (1996) Phage Display of Peptides and Proteins: A Laboratory Manual, Academic Press, San Diego, CA; de Bruin et al. (1999) Nature Biotechnol. 17:397-399).

Single chain antibodies and diabodies are described (see, e.g., Malecki et al. (2002) Proc. Natl. Acad. Sci. USA 99:213-218; Conrath et al. (2001) J. Biol. Chem. 276:7346-7350; Desmyter et al. (2001) J. Biol. Chem. 276:26285-26290; Hudson and Kortt (1999) J. Immunol. Methods 231:177-189; and U.S. Pat. No. 4,946,778). Bifunctional antibodies are provided (see, e.g., Mack, et al. (1995) Proc. Natl. Acad. Sci. USA 92:7021-7025; Carter (2001) J. Immunol. Methods 248:7-15; Volkel, et al. (2001) Protein Engineering 14:815-823; Segal, et al. (2001) J. Immunol. Methods 248:1-6; Brennan, et al. (1985) Science 229:81-83; Raso, et al. (1997) J. Biol. Chem. 272:27623; Morrison (1985) Science 229:1202-1207; Traunecker, et al. (1991) EMBO J. 10:3655-3659; and U.S. Pat. Nos. 5,932,448, 5,532,210, and 6,129,914).

Bispecific antibodies are also provided (see, e.g., Azzoni et al. (1998) J. Immunol. 161:3493; Kita et al. (1999) J. Immunol. 162:6901; Merchant et al. (2000) J. Biol. Chem. 74:9115; Pandey et al. (2000) J. Biol. Chem. 275:38633; Zheng et al. (2001) J. Biol Chem. 276:12999; Propst et al. (2000) J. Immunol. 165:2214; Long (1999) Ann. Rev. Immunol. 17:875).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2nd ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probes (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, MD); GCG Wisconsin Package (Accelrys, Inc., San Diego, CA); DeCypher® (TimeLogic Corp., Crystal Bay, Nevada); Menne, et al. (2000) Bioinformatics 16:741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### EXAMPLES

The following examples serve to illustrate the present invention. These examples are in no way intended to limit the scope of the invention.

### EXAMPLE 1: REAGENTS

The details of reagents and antibodies used for the following examples are provided in Table 1:

| | Dilutions/ | | Catalog | |
|---|---|---|---|---|
| Antibody/reagents | concentration | | number/clone | Vendor |
| IL-2 | 6000 | U/mL | Proleukin | Novartis |
| recombinant TGF | 10 | ng/mL | 100-21C | Peprotech |
| PHA | 10 | μg/mL | L1668-5MG | Sigma Aldrich |
| Recombinant E-cadherin (1) | 2 | μg/mL | 648-EC | R & D systems |
| Recombinant E-cadherin (1) | 2 | μg/mL | 10204-H02H | Sino Biological |
| CD103 (2G5.1) antibody | 10-20 | μg/mL | MCA708 | Bio-rad |
| CD103 (Ber-ACT8) | 10-20 | μg/mL | 550258 | BD |
| antibody | | | | |
| CD103 (Ber-ACT8)- | 15 μL per test | | 561677 | BD |
| FITC antibody | | | | |
| CD3-PE antibody | 2 μL per test | | 12-0038-41/42 | eBioscience |
| CD8a-APC- | 2 μL per test | | 47-0088-42 | eBioscience |
| eFlour 780 antibody | | | | |
| CD33-PE-Cy7 antibody | 2 μL per test | | 25-0338-42 | eBioscience |
| CD3-PerCP- | 2 μL per test | | 45-0037-42 | Thermo Fisher |
| Cy5.5 antibody | | | | Scientific |
| CD324 (E-Cadherin)- | 1 | μg/test | 46-3249-82 | eBioscience |
| PerCP-eFluor 710 antibody | | | | |
| Mouse IgG1 κ | 10-20 | μg/mL | 554721 | BD biosciences |
| Isotype Control | | | | |
| Human IgG, | 10-20 | μg/mL | 009-000-007 | Jackson |

| | | | |
|---|---|---|---|
| Fab fragment | | | Immunoresearch |
| Zombie Aqua ™ Fixable | 1:100 | 423102 | BioLegend |
| Viability kit | | | (ITK diagnostics) |
| Goat anti-mouse Ig-PE | 1:50 | 1010-9 | Southern Biotech |
| F(ab′)2 Fragment Goat | 1:50 | 109-136-088 | Jackson |
| Anti-Human IgG | | | Immunoresearch |
| (H + L)-APC | | | |
| Murine CD3- | 2 μL per test | 11-0032-82 | eBioscience |
| FITC antibody | | | |
| Murine CD8a- | 2 μL per test | 25-0081-82 | eBioscience |
| PE-Cy7 antibody | | | |
| Murine CD | 2 μL per test | 12-1031-82 | eBioscience |
| 103-PE antibody | | | |

### EXAMPLE 2: PRIMARY MATERIAL AND CELL LINES

Chinese hamster ovary (CHO)-K1 cells and the human adenocarcinoma cell line MCF7 were obtained from the American Type Culture Collection (ATCC). Cells were quarantined until screening for microbial contamination and mycoplasma was performed and proven to be negative. CHO-K1 cells were grown in DMEM/F12 (Gibco), 1% PenStrep (Gibco), 5% NCBS (Biowest) and incubated in a humidified atmosphere with 5% CO₂ at 37° C. MCF7 cells were grown in EMEM (ATCC), 1% PenStrep (Gibco), 10% FBS (Gibco) and incubated in a humidified atmosphere with 5% CO₂ at 37° C. A CHO-K1.hCD103/hBeta7 cell line was generated by transfecting CHO-K1 cells with pCI-neo and pcDNA3.1 (+)-hygro vectors encoding the full length open reading frames of human integrin AlphaE (UniProt P38570) and human integrin Beta 7 (UniProt P26010), respectively. Stable clones were obtained by limiting dilution in CHO medium supplemented with geneticin (50 ug/mL, Gibco) and hygromycin B (50 μg/mL, Invitrogen). CHO-K1.hAlpha4/hBeta7 expressing cells were generated by transient transfection of CHO-K1 cells with pCI-neo and pcDNA3.1 (+)-hygro vectors encoding the full length open reading frames of human integrin Alpha4 (UniProt P13612) and human integrin Beta 7, respectively. CHO-K1.rhCD103/rhBeta7 expressing cells were generated by transient transfection of CHO-K1 cells with pCI-neo and pcDNA3.1 (+)-hygro vectors encoding the full length open reading frames of rhesus integrin AlphaE (UniProt H9Z8N2) and rhesus integrin Beta 7 (NCBI XP_015007317.1), respectively.

The human non-small cell lung cancer cell line A549 was obtained from the American Type Culture Collection (ATCC). Cells were quarantined until screening for microbial contamination and mycoplasma was performed and proven to be negative. Cells were grown in DMEM/F-12, GlutaMAX™ Supplement+5% FCS+25 mM HEPES for CHO-K1 and RPMI+10% FCS for A549, and incubated in a humidified atmosphere with 5% CO₂ at 37° C. The A549 cell line was subjected to knockout of CDH1 (E-cadherin) by nonliposomal transfection (Fugene) using a plasmid encoding guide RNAs, a fully functional CAS9 cassette and GFP (plasmid pSpCas9 (BB)-2A-GFP (PX458) (Ran et al. Nature Protocols 8:2281-2308 (2013)) (Addgene plasmid #48138; n2t.net/addgene: 48138; RRID: Addgene_48138)). GFP-positive single-cell clones were isolated using a Moflo Astrios sorter (Beckman Coulter). Disruption was confirmed by Sanger sequencing with tracking of indels.

CD103 positive T cells were generated as follows. Human peripheral blood mononuclear cells (PBMC) were isolated via Ficoll-Paque density gradient centrifugation (Ficoll-Paque PLUS, GE Healthcare Life Sciences, Marlborough, MA, USA) of buffy coats from healthy volunteers after informed consent (Sanquin). Next, CD8 positive T cells were negatively selected using a MagniSort™ Human CD8 T cell Enrichment Kit according to standard protocol (Thermo Fisher Scientific). Subsequently, cells were stimulated with 10 μg/mL PHA, 6000 U/mL IL-2 and 10 ng/mL recombinant TGFβ, and cultivated in RPMI supplemented with 10% FCS and penicillin/streptomycin (100 U/mL). Cells were cultured for at least 10 days to obtain >80% CD103 positive CD8 cells.

Fresh tumor material was obtained from ovarian cancer patients undergoing cytoreductive surgery. With a scalpel, tumor pieces of approximately 1 mm³ were cut, and subjected to enzymatic digestion (RPMI supplemented with 1 mg/ml collagenase type IV (Life technologies), 31 U/ml rhDNase (Pulmozyme, Genentech, California, USA) and 10% FCS) for 30 minutes at 37° C. or overnight at room temperature. Subsequently, the digestion medium containing remaining tumor pieces was filtered over a 70 μm cell strainer (Corning, Amsterdam, The Netherlands). For flow cytometric analyses, cells were pelleted, washed, and cryopreserved until further use.

Spleens from immunocompentent Balb/c and C57/BL6 mice, and the thymus from Balb/c mice were harvested, followed by mincing of the tissue on a 70 μm strainer with a plunger. Red blood cells were removed using Red Blood Cell Lysis Buffer (Biolegend). Cells were pelleted, washed, and cryopreserved until further use.

### EXAMPLE 3: MONOCLONAL ANTIBODY GENERATION

To generate human CD103 antibodies, mice were immunized with the cDNA plasmid constructs encoding full length open reading frames of human CD103 (integrin alpha-E) and human integrin beta-7. The pCI-neo and pcDNA3.1 (+) were custom-based synthesized and obtained from GeneArt/ThermoFisher (Regensburg, Germany). Mice were immunized by gene gun immunization using a Helios Gene gun (BioRad, Hercules, CA, USA) and DNA coated gold bullets (BioRad) following manufacturer's instructions at Envigo (Horst, The Netherlands). Briefly, 1 μm gold particles were coated with pCI-neo-hCD103 and pcDNA3.1 (+)-hBeta7 cDNA and commercial expression vectors for mouse Flt3L and mouse GM-CSF (both from Aldevron) in a 1:1:1:1 ratio. A total of 50 μg of plasmid DNA was used to coat 25 mg of gold particles. Specifically, 7-8 weeks old female BALB/C mice (Harlan) were immunized in the ears with a gene gun, receiving 3 administration cycles in both ears.

Antibody titer was assessed by cell ELISA (“CELISA”), using a CHO-K1.hCD103/hBeta7 stable cell line. Cells were seeded into 96-well flat-bottom tissue culture plates at 8×10⁴ cells/well and cultured at 37° Celcius, 5% CO2 and 95% humidity until cell layers were confluent. Cells were incubated with each sample of the diluted mouse sera for 1 hour at 37° C., 5% CO2 and 95% humidity. Next, cells were washed with phosphate buffered saline (PBS)/0.05% Tween-20 (PBS-T) and incubated with goat-anti-mouse IgG-HRP conjugate (Southern Biotech) for 1 hour at 37° C., 5% CO2 and 95% humidity. Subsequently, cells were washed three times with PBS-T and anti-hCD103/hBeta7 immunoreactivity was visualized with TMB Stabilized Chromogen (Invitrogen). Reactions were stopped with 0.5 M H₂SO₄ and absorbances were read at 450 and 610 nm. The anti-hCD103/hBeta7 titer was higher than 1:2500 in each individual mouse serum sample as detected after two DNA immunizations. All mice were immunized for a final, third time and sacrificed 4 days later. Erythrocyte-depleted spleen and lymph-node cell populations were prepared according to published protocols.

To select anti-hCD103 antibody producing B-cells, a selection strategy was designed and developed that preferentially bound B-cells expressing antibodies that bind specifically to hCD103, preferably with cross-reactivity to monkey CD103. As cynomolgus CD103 sequences were not known, cross-reactivity studies were performed using rhesus CD103. Splenocytes and lymphocytes from the hCD103/hBeta7 immunized mice were incubated with hCD103 negative MCF-7 that were seeded into T25 culture flasks and irradiated at 30 Gray. After 1 hour unbound cells were gently removed by moving the flask back and forth. Medium containing unbound cells was then transferred to a new T25 flask containing irradiated CHO-K1.hAlpha4/hBeta7 cells (transient transfection). This procedure was repeated one more time on ice in order to negatively select hBeta7-reactive B-cells. Next, medium containing unbound B-cells was incubated with CHO-K1.hCD103/hBeta7 cells that were irradiated with 30 Gy. After 1.5 hours incubation on ice unbound cells were removed with multiple wash steps using culture medium. Subsequently, T25 flasks containing CHO-K1.hCD103/hBeta7 cells with bound lymphocytes were harvested with Trypsin-EDTA (Sigma). Selected B-cells were mixed with 10% (v/v) T-cell supernatant and 50,000 irradiated (25 Gy) EL-4 B5 feeder cells in a final volume of 200 μl medium in 96-well flat-bottom tissue culture plates. On day four, cell culture medium was refreshed. On day eight, supernatants were screened for hCD103/hBeta7 reactivity by cell ELISA as described below. CHO-K1.hCD103/hBeta7, CHO.K1.rhCD103/rhBeta7 (transient transfection) and CHO-K1.hAlpha4/hBeta7 (transient transfection) were seeded in culture medium (DMEM-F12 (Gibco) supplemented with 10% Fetal Bovine Serum (Hyclone) and 80 U Pen/Strep (Gibco)) in 96-well flat-bottom tissue culture plates and cultured at 37° C., 5% CO2 and 95% humidity until they were confluent. Subsequently, culture medium was removed and cells were incubated for 1 hour at 37° C., 5% CO₂ and 95% humidity with supernatants from the B-cell cultures. Next, cells were washed with PBS-T and incubated for 1 hour at 37° C., 5% CO2 and 95% humidity with goat-anti-mouse IgG-HRP conjugate (Southern Biotech). Subsequently, cells were washed three times with PBS-T and anti-hCD103/hBeta7, and anti-hAlpha4/hBeta7 immunoreactivity was visualized with TMB Stabilized Chromogen (Invitrogen). Reactions were stopped with 0.5 M H₂SO₄ and absorbances were read at 450 and 610 nm.

B-cell clones from the hCD103/hBeta7 reactive supernatants, which were not or which were minimally reactive to hAlpha4/hBeta7 were immortalized by mini-electrofusion following a published procedure (Steenbakkers et al. (1992) Mol. Biol. Rep. 19:125) with some minor deviations. Briefly, B-cells were mixed with 106 Sp2/0-Ag14 murine myeloma cells (ATCC CRL-1581) in Electrofusion Isomolar Buffer (Eppendorf). Electrofusions were performed in a 50 μL fusion chamber by an alternating electric field of 15 s, 1 MHz, 23 Vrms AC followed by a square, high field DC pulse of 10 as, 180 Volt DC and again by an alternating electric field of 15 s, 1 MHz, 23 Vrms AC. Content of the chamber was transferred to hybridoma selective medium and plated in a 96-well plate under limiting dilution conditions. On day 8 following the electrofusion, hybridoma supernatants were screened for hCD103/hBeta7, rhCD103/rhBeta7, and hAlpha4/hBeta7 binding activity by cell ELISA as described above. Hybridomas that secreted antibodies in the supernatant that specifically bound CD103 were frozen at −180° C. (−1 batch) and subcloned by limited dilution to safeguard their integrity and stability. Stable hybridomas were frozen at −180° C. (−LD1 batch) until cell layers were confluent.

Selected stable hybridomas were cultured in serum-free media for 7 days; supernatants were harvested and antibodies were purified using MabSelect Sure Protein A resin according to the manufacturer's instructions (GE Healthcare). Antibody concentrations were quantified using spectrophotometry. Antibody monomericity was assessed by SEC-HPLC. Supernatants of the hybridoma cultures were used to isotype the hybridomas. In short, isotyping was done using a mouse monoclonal antibody isotyping kit (Biorad) based on a dipstick with immobilized goat-anti-mouse antibody bands to each of the common mouse isotypes and light chains. Recovered antibodies were all identified as mouse IgG1. Antibody sequences were elucidated by sequencing of variable regions of the mouse IgG 1 hybridoma material performed at LakePharma (CA, USA), using the following method: the total RNA of the hybridoma cells was extracted, which allowed cDNA synthesis. Rapid Amplification of cDNA Ends (RACE) was performed that allowed cloning of positive fragments in a TOPO (Thermo Fisher Scientific) vector. TOPO clones were sequenced and sequences were annotated using VBASE2.

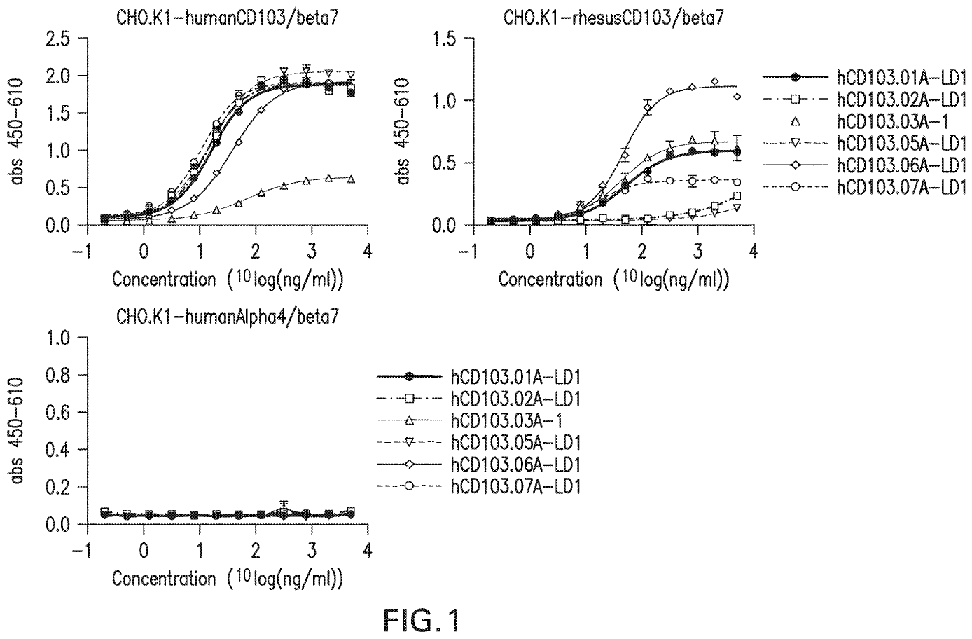

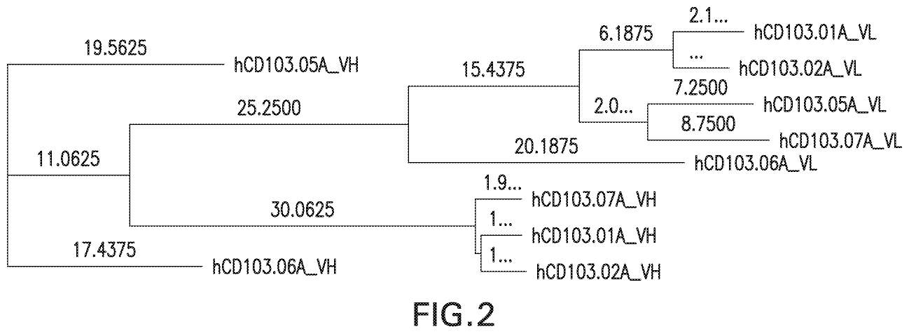

The mAb discovery campaign yielded 6 different mAb candidates that showed specific binding to the human CD103 (integrin alpha-E) domain of the integrin heterodimer hCD103/hBeta7. The 6 selected candidates were produced from hybridoma and purified. FIG. 1 presents the cell binding data of the purified anti-hCD103 mAbs to CHO.K1-hCD103/hBeta7, CHO.K1-rhCD103/rhBeta7, and CHO.K1-hAlpha4/hBeta7. Expression of the integrin heterodimers by the transfected CHO-K1 cell lines was confirmed by commercial mAbs against integrin human CD103, integrin human Alpha-4, and integrin human Beta-7. Selected hybridomaswere sequenced and a phylogenetic tree was built using Discovery Studio (FIG. 2), showing that all VH and VL sequences are unique with different degrees of similarity. As shown, the antibodies exhibit binding to hCD103, but no binding to hBeta7. Binding to rhesusCD103/rhBeta7 is different for the various candidates

### EXAMPLE 4: GENERATION OF ANTI-HCD103 FAB FRAGMENTS

Anti-hCD103 Fab candidates were produced by ImmunoPrecise (Oss, the Netherlands). Synthetic vectors encoding for the DNA sequences of the V_{H} and V_{L} domains of candidates hCD103.01A, hCD103.05A, and hCD103.06A were synthesized and subsequently cloned into ImmunoPrecise's human IgG1-Fab-K vector and human kappa light chain vector, respectively, followed by transfection of HEK293 cells. Fab fragments from harvested supernatants were purified by endotoxin-free purification using CaptureSelect IgG-CH1 affinity matrix. Fab concentrations were quantified using Spectrophotometry and Fab purity was assessed by SDS-PAGE and HP-SEC. Endotoxin levels were determined by LAL assay.

### EXAMPLE 5: FLUORESCENT LABELLING OF MABS AND FABS

Fabs and mAbs were conjugated with a 6× molar excess of Alexa Fluor 647-NHS (Thermo Scientific). In short, mAbs/Fabs were rebuffered to 0.2M sodium bicarbonate pH 8.3 using Zeba 7K MWCO spin columns. A 6-fold molar excess of Alexa Fluor 647-NHS (from a 10 mg/mL stock in DMSO) was added. The reaction was allowed for 1 h in the dark at room temperature. Non-reacted Alexa Fluor-NHS was removed using Zeba 7K MWCO spin columns. Antibody and AF647 concentrations were measured using spectrophotometry at 280 nm and 650 nm, respectively. The amount of residual non-reacted AF647 was determined by HP-SEC using a dual detector system (280 nm and 650 nm). Labeling yields of +/−4 dyes per mAb and +/−2-3 dyes per Fab were observed.

### EXAMPLE 6: IMMUNOREACTIVITY TO CHO CELLS EXPRESSING HUMAN CD103/BETA7

Non-labeled mAbs and Fabs were analysed for cellular binding to CHO.K1-hCD103/hBeta7 by CELISA as described earlier. Next, cellular binding experiments were also performed using flow cytrometry to determine the binding profiles of the AF647 labeled mAbs/Fabs on CHO.K1-hCD103/hBeta7 and CHO.K1. 1×10⁵ detached cells were incubated with mAb/Fab at 4° C. for 30 min. After washing, the cells were resuspended in 1% BSA/DPBS/1×DAPI and analysed on the FACS-CantoII (BD Biosciences).

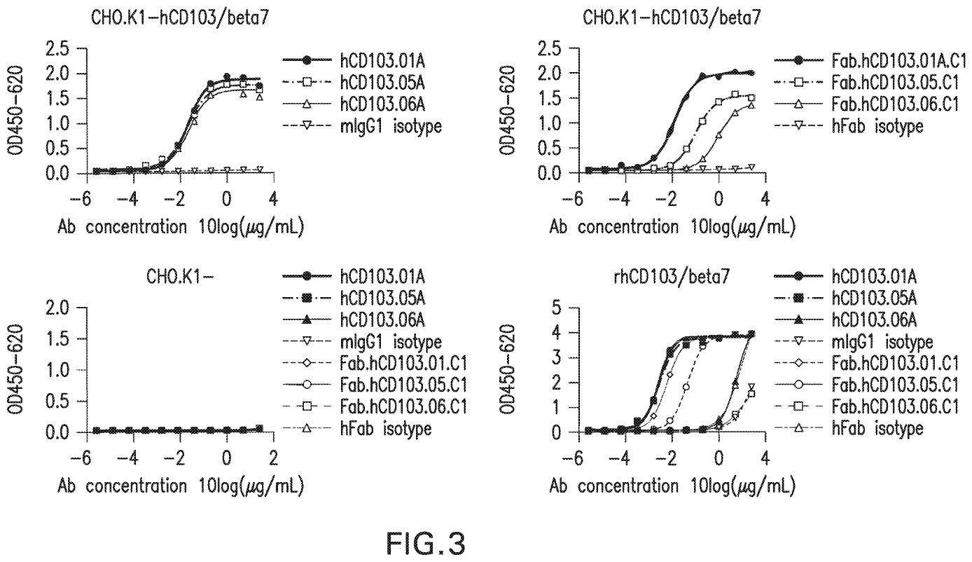

FIG. 3 presents the binding data of the various non-labeled mAbs/Fabs in cell ELISA. Potent binding of hCD103.01 mAb/Fab and hCD103.05 mAb to both CHO.K1.hCD103/beta7 and recombinant hCD103/beta7, somewhat reduced binding of hCD103.05 Fab to both CHO.K1.hCD103/beta7 and recombinant hCD103/beta7 and weak/minimal binding of hCD103.06 mAb/Fab to recombinant hCD103/beta7 (Acro Biosystems), while strong binding of hCD103.06 mAb to CHO.K1.hCD103/hBeta7

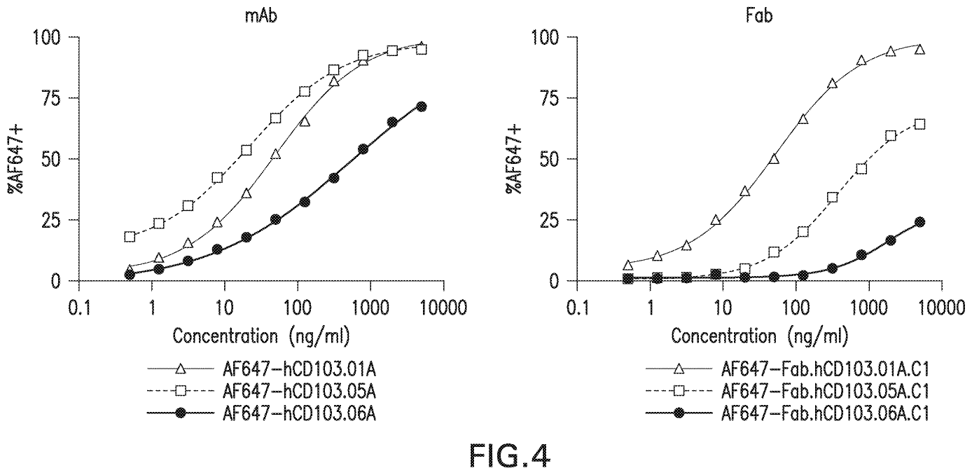

FIG. 4 presents the binding data of the various AF647-labeled mAbs/Fabs in flow cytometry. No dose dependent binding of the mAb and Fab reagents was observed on non-transfected CHO.K1 (data not shown).

### EXAMPLE 7: IMMUNOREACTIVITY TO RECOMBINANT HUMAN CD103/BETA7

Immunoreactivity to human CD103/Beta7 was assessed by ELISA using recombinant hCD103/hBeta7 Fc-protein (Acro-Biosystems) coated 96-well MaxiSorp flat-bottom plates. Protein coated 96-well plates were blocked in protein-free blocking buffer (Pierce) for 1.5 hour at 37° C. Plates were washed and incubated for 1 hour at RT with mAb/Fab in 0.1% Tween-20 in PBS. Next, plates were washed with PBS-T and incubated for 1 hour at RT with goat-anti-mouse IgG-HRP conjugate (Southern Biotech) for mAbs, and goat-anti-human Fab-HRP conjugate (Jackson Immuno Research) for the Fab fragments in 0.1% Tween-20 in PBS. Subsequently, wells were washed three times with PBS-T and anti-hCD103 immunoreactivity was visualized with TMB Stabilized Chromogen (Invitrogen). Reactions were stopped with 0.5 M H2SO4 and absorbances were read at 450 and 610 nm.

### EXAMPLE 8: FLOW CYTOMETRY ANALYSIS

For binding assays of anti-CD103 mAbs and Fab fragments in tumor digests, samples were divided in multiple aliquots and stained using either a live/dead marker and commercial antibodies against human CD3, CD8a, CD33 and CD103, or commercial antibodies against CD3, CD8a, CD33 and our anti-CD103 mAbs or Fabs with secondary detection reagent. For binding assays of anti-CD103 with murine CD103 positive T cells spleen and thymus single cells suspensions were divided in multiple aliquots and stained using either a live/dead marker and commercial antibodies against murine CD8 and CD103. Additional aliquots were stained with relevant isotype controls or as fluorescence minus one controls (“FMO” controls are cells stained with all the fluorophores minus one fluorophore; the relevant isotype controls contain either a directly labeled unspecific isotype antibody or an unspecific isotype antibody combined with secondary detection reagent). Percentage binding of fluorescently labeled mAbs was determined using flow cytometry. Maximum binding was set at 100%. Measurement was performed on a BD FACSVerse (BD Biosciences). Data analysis was performed with FlowJo v10 (Tree Star) and surface receptor levels were expressed as mean fluorescent intensity (MFI).

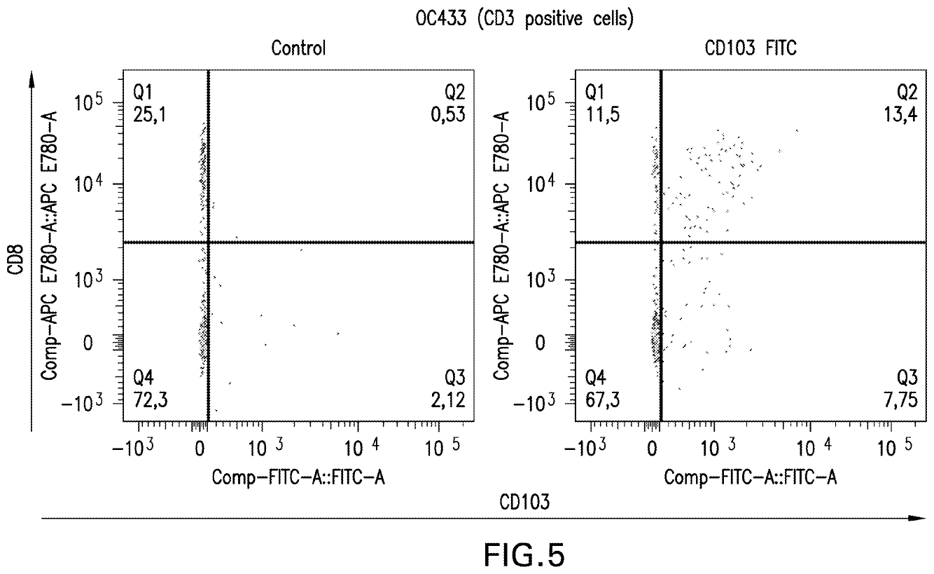

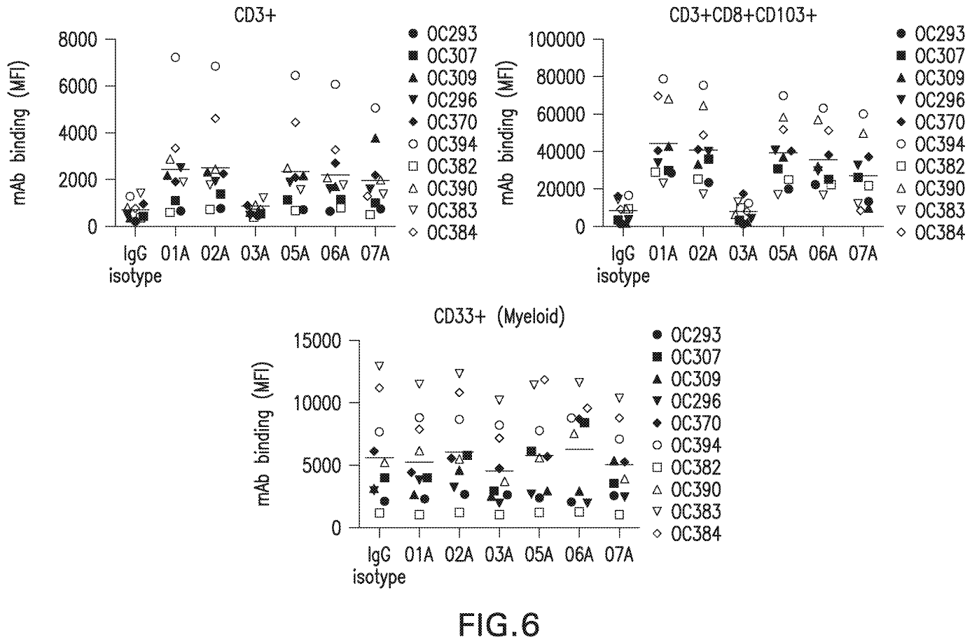

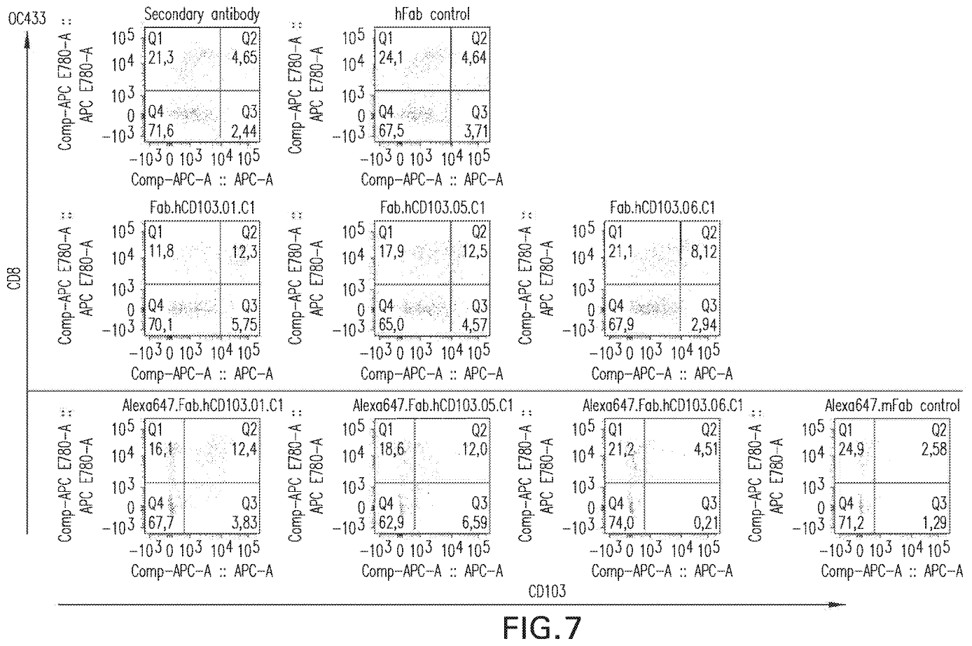

Using ex vivo human tumor digests, the anti-CD103 mAbs were assessed against a benchmark commercial anti-CD103 mAb (BD bioscience) routinely used in flow cytometry, gating on CD3+CD8+ T cells. The anti-CD103 mAbs readily identified the CD103+ CD8+ T cell subpopulation at frequencies identical to that observed for the commercial anti-CD103 mAb (FIG. 5). No binding to CD4+ T cells or CD33+ (myeloid) cells was detected in these digests. mAb binding to the CD103+ CD8+ T cell subpopulation in ten independent patients was highest for clone 01A, whereas clone 03A showed the lowest binding (FIG. 6). Additionally, recombinant Fab fragments from antibody clone 01A, 05A and 06A. showed respectively the strongest binding to the CD103+ CD8+ T cell subpopulation for Fab.hCD103.01.C1, whereas Fab.hCD103.06.C1 showed the lowest binding in human tumor digests (FIG. 7), in line with the mAb binding data.

To assess differences in affinity and competition between the mAbs, CD103+CD8+ T cells were pre-incubated with our anti-CD103 mAbs or the commercial anti-CD103 mAbs in FACS medium for 1 hour at 4° C. and subsequently incubated with their fluorescently labeled counterparts for 1 hour at 4° C. Percentage binding of fluorescently labeled mAbs was determined using flow cytometry. Maximum binding was set at 100%

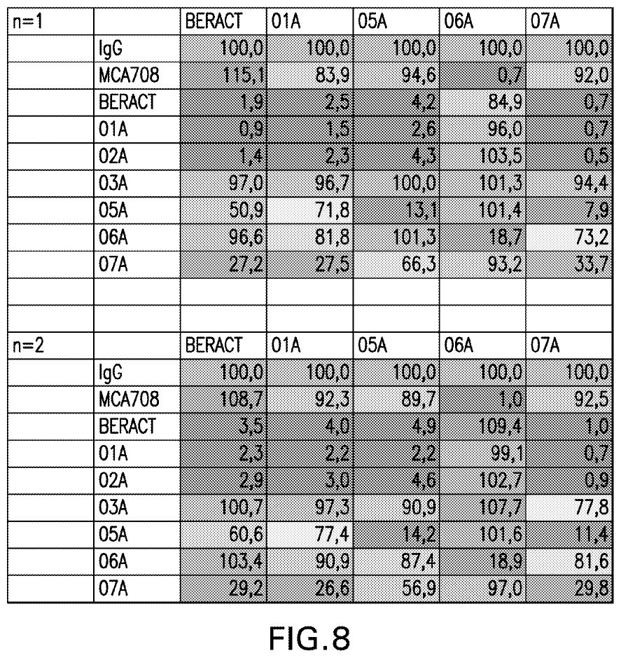

Flow cytometry was used to determine whether the CD103 mAb clones cross-react with murine CD103+ T cells. Mouse spleen and thymus were examined for expression of CD103 using a commercial anti-mouse CD103 mAb. Approximately half of the CD8 cells were positive for CD103. However, the anti-human CD103 mAb clones showed no specific binding to murine CD103 (data not shown). Competition assays revealed that binding of most of the mAbs in our panel, except clone 03A and 06A, inhibited binding of the commercial CD103 mAb and vice-versa, indicating binding to the same region on CD103 (FIG. 8). Nevertheless, differences in binding characteristics were observed. Clone 01A and 02A blocked binding of most other mAb clones in a competition assay, whereas clone 03A, 05A, 06A and 07A did not, suggesting distinct binding epitopes. Additionally, fluorescently labeled 05A, 06A and 07A showed binding after saturation with the same clone, indicating lower binding affinities.

Internalization and dissociation of anti-CD103 mAbs and Fab fragments, and membranous turnover of CD103 were determined using a previously described protocol. Briefly, CHO.K1-hCD103/hBeta7 cells or CD103 positive T cells were stained on ice with the anti-CD103 mAbs and Fab fragments (20 μg/mL final concentration). After staining; 1) cells were washed with ice-cold FACS buffer and incubated with secondary antibody diluted 1:50 in FACS medium for 1 hour at 4° C. to measure surface expression. 2) Cells were washed with ice-cold FACS buffer, incubated in culture medium at 37° C. for 4 hours and subsequently incubated with secondary antibody for 1 hour at 4° C. to measure non-internalized CD103-antibody complexes since the secondary antibodies only bind to surface bound CD103 mAbs or Fab fragments. 3) Cells were washed with ice-cold FACS buffer, incubated in culture medium at 37° C. for 4 hours and subsequently re-incubated with the CD103 mAbs or Fab fragments, followed by secondary antibody to measure non-internalized, reappeared receptors and possible de novo synthesis of receptors. Duplicate samples were measured for each treatment condition, and corrected for background fluorescence and unspecific binding of the secondary antibody. Measurement was performed on a BD FACSVerse or BD Accuri C6 (BD Biosciences). Data analysis was performed with FlowJo v10 (Tree Star) and surface receptor expression was expressed as mean fluorescent intensity (MFI). Four hours incubation at 37° C. resulted in different amounts of remaining mAbs and Fab fragments bound at the cell surface. A decrease could indicate internalized mAb and Fab fragments. However, performing the same experiment using directly labeled mAbs and Fab fragments showed that the decrease in remaining mAb or Fab fragment at the cells surface was due to dissociation. Of note, CD103 surface expression levels were only marginally changed by the incubation with mAbs or Fab fragments (data not shown)

### EXAMPLE 9: CD103+T CELL ADHESION ASSAYS

CD103⁺ T cell adhesion assays were performed as follows. One day before the experiment, 96 wells plates were coated overnight at 4° C. with 100 μL recombinant E-cadherin at 2 μg/mL in Dulbecco's PBS (DPBS) containing 1 mM Ca2⁺ and Mg2⁺. Next, wells were blocked for at least 1 hour using 1% bovine serum albumin (BSA) in DPBS. CD103⁺ T cells were labeled with CFSE (Thermo Fisher Scientific) as described earlier (3) and resuspended in RPMI+10% FCS+1 mM Mn²⁺. CFSE labeled cells were either preincubated with 10 μg/mL antibody or Fab fragments for 30 minutes on ice followed by incubation in E-cadherin coated wells (50,000 cells/well) for 30 minutes at 37° C. or cells were directly transferred to E-cadherin coated wells for 30 minutes at 37° C. followed by 10 μg/mL antibody or Fab fragment treatment for 30 minutes at 37° C.

For adhesion assays using A549 wild-type and E-cadherin knock-out cells, one day before the experiment, tumor cells (30,000 cells/well) were seeded in 96 wells plates. Next, CFSE labeled CD103⁺ T cells were preincubated with 10 μg/mL antibody for 30 minutes on ice followed by incubation in tumor cell seeded wells for 60 minutes at 37° C.

After incubation, unbound cells were removed by inverting the plate and washing with DPBS. Finally, cells were fixed using 3.7% formalin in DPBS. Images were captured using a conventional fluorescent microscope (Invitrogen™ EVOS™ FL Imaging System). Bound T cells were quantified using Image J software analysis (1.50v).

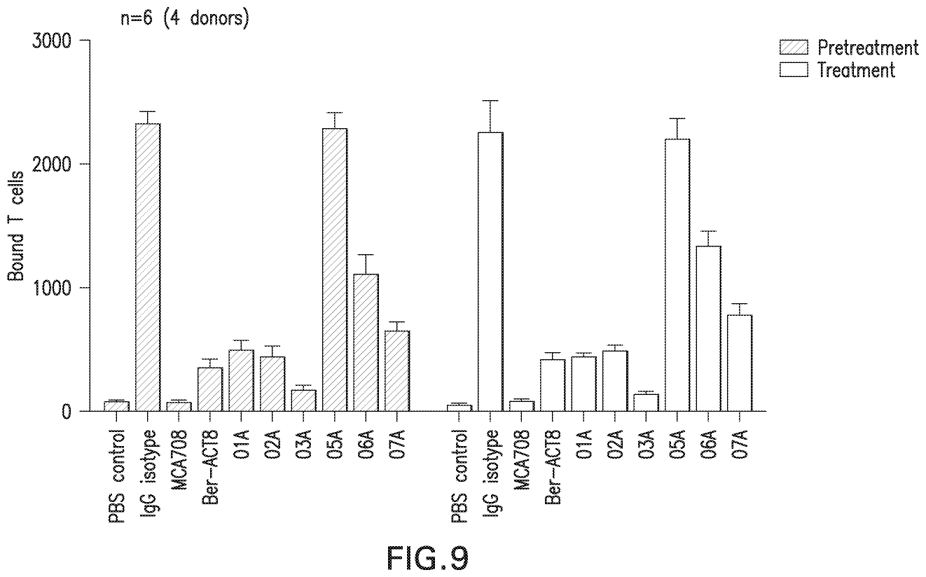

As shown in FIG. 9, CD103 mAb clone 01A, 02A, 03A and 07A showed the strongest inhibition of T cell binding, whereas 06A partially inhibited the binding to E-cadherin. Clone 05A was the only clone that did not interfere with CD103-mediated T cell adhesion. Similar effects were observed for Fab.hCD103.01.C1, Fab.hCD103.05.C1, and Fab.hCD103.06.C1 (data not shown). CRISPR-knockout of CDH1 (E-cadherin) in A549 tumor cells resulted in reduced CD103⁺ T cell adhesion, however no clear effects of our CD103 mAbs were observed in E-cadherin wild-type and E-cadherin-knockout cells.

### EXAMPLE 10: CELL BASED ELISA

One day before the procedure, CD103/B7 transfected CHO cells (30,000 cells/well) were seeded in 96 wells plates. Subsequently, serial dilutions of CD103 mAbs, Fab fragments and isotype controls were added to each well of a 96-well plate and incubated for 1 h at 37° C. Wells were washed with PBS and incubated with Rabbit anti-Mouse/IgG-HRP (1:4000, Dako) or Fab specific Goat anti-Human/IgG-HRP (1:4000; Sigma Aldrich) for 1 h at 37° C. Next, wells were washed with PBS and TMB substrate (KPL) was added. The color reaction was stopped by adding 1M HCl solution and the absorbance was measured by a microplate reader (Thermo Scientific).

### EXAMPLE 11: 89ZR-HCD103.01A, ⁸⁹ZR-HCD103.05A, ⁸⁹ZR-FAB.HCD103.01.C1 AND ⁸⁹ZR-HCD103.05.C1 TRACER DEVELOPMENT AND QUALITY CONTROL

hCD103.01A, hCD103.05A, Fab.hCD103.01.C1 and Fab.hCD103.05.C1 were incubated with a 3 or 4-fold molar excess of TFP-N-Suc-desferal-Fe (Df, ABX GmbH, Hamburg, Germany) and subsequent ⁸⁹Zr-labeling was performed using clinical grade ⁸⁹Zr (Perkin Elmer, Groningen, The Netherlands). Maximal attainable specific activity was determined using varying amounts of ⁸⁹Zr per mg antibody or Fab fragment ranging between 250 and 1000 MBq/mg. Radiochemical purity (RCP) was assessed by trichloroacetic acid (TCA) precipitation test. Radiochemical purity for ⁸⁹Zr-Fab.hCD103.01.C1 and Fab.hCD103.05.C1 was >96% for three ⁸⁹Zr levels tested (250, 500, and 750 MBq ⁸⁹Zr). Df-mAb and -Fab conjugates were checked for aggregation and fragmentation by size exclusion ultra-performance liquid chromatography (SE-UPLC). The Waters SE-UPLC system was equipped with a dual wavelength absorbance detector, in-line radioactivity detector and TSK-GEL G3000SWXL column (JSB, Eindhoven, The Netherlands).

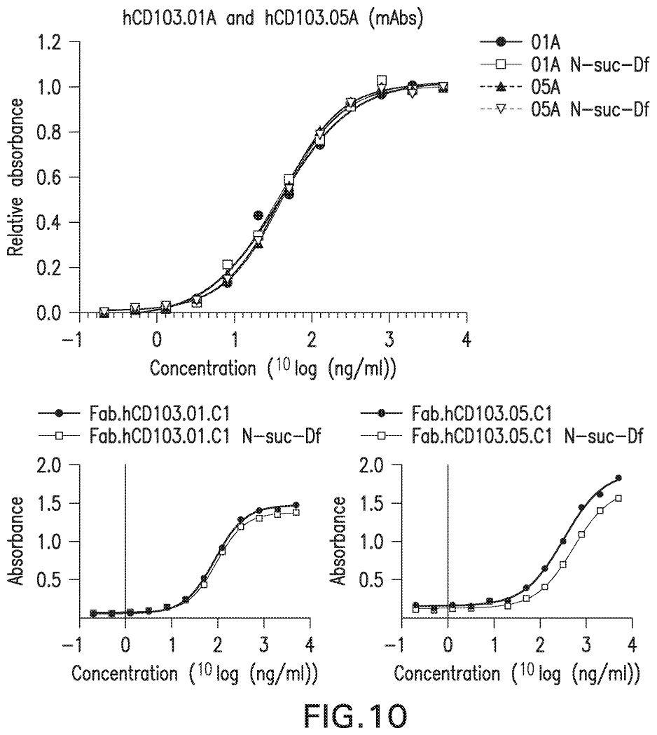

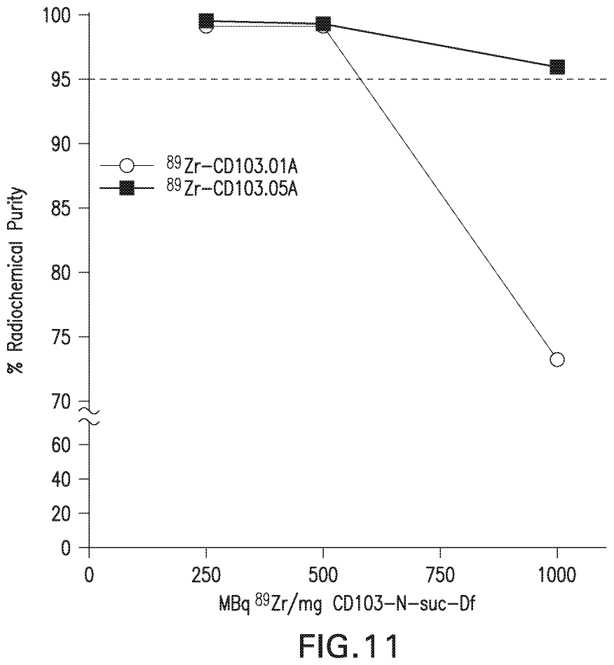

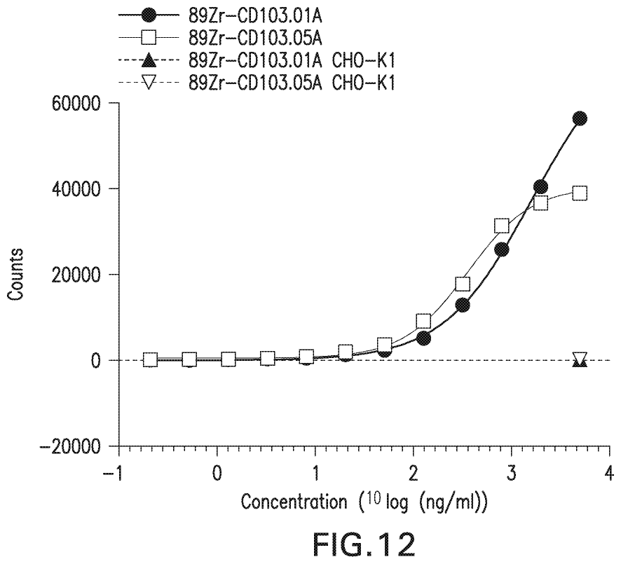

CD103 binding affinity of the two Df-conjugated CD103 mAbs and Fab fragments was similar to their unmodified counterparts (FIG. 10). In addition, both the Df-conjugated mAbs and the Fab fragments achieved a specific activity of 500 MBq ⁸⁹Zr/mg at a radiochemical purity of >95%, without further purification (FIG. 11). As such, these tracers are suitable for PET imaging with amounts as low as 10 μg (PET imaging) or even less when used in biodistribution studies. In vitro, hCD103.01A and hCD103.05A showed specific binding to a CD103 transfected CHO-K1 model cell line (CHO.K1-hCD103/hBeta7), but not to CHO-K1 wild type cells (CHO.WT) (FIG. 12).

### EXAMPLE 12: ANIMAL STUDIES

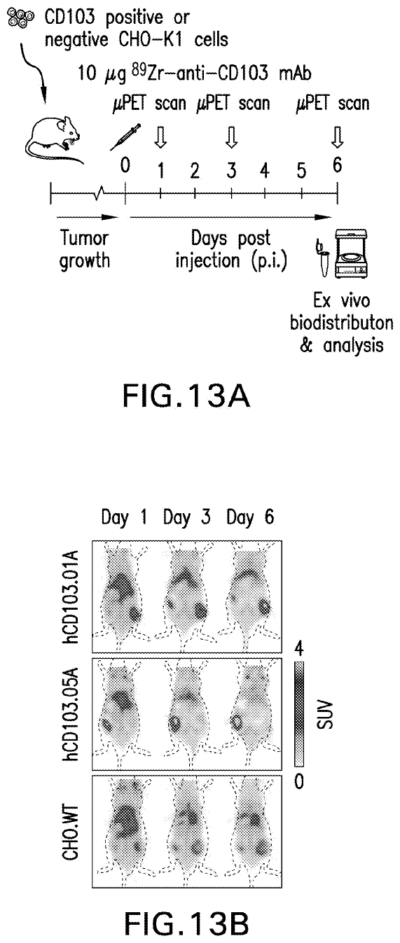

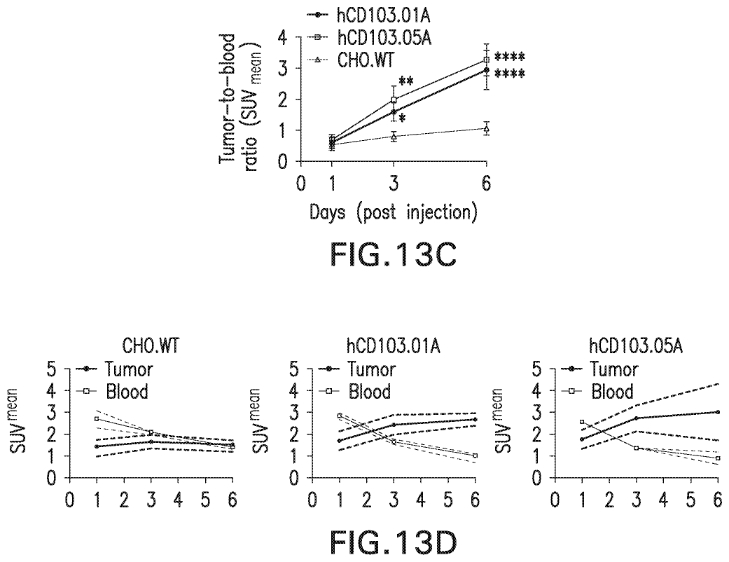

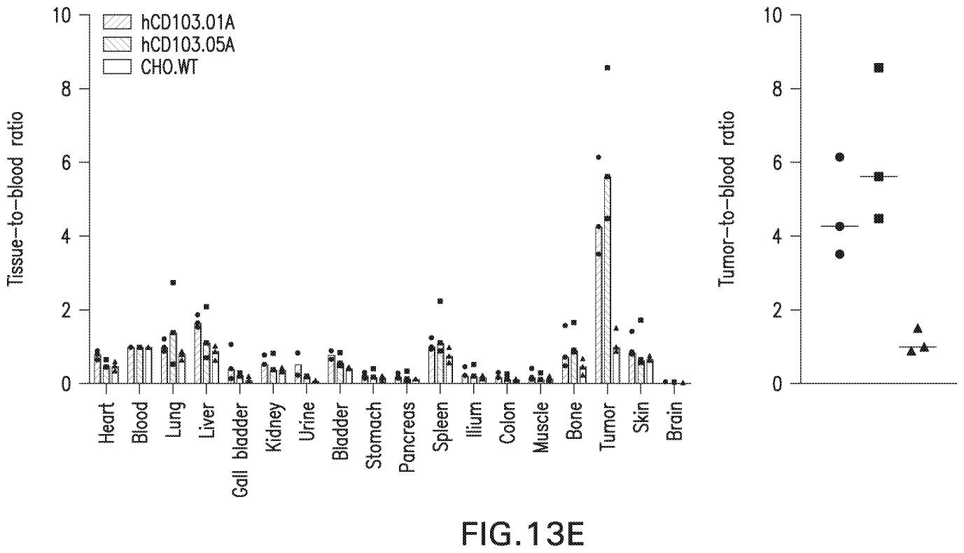

FIG. 13A depicts an exemplary PET imaging protocol for ⁸⁹Zr-hCD103.01A and ⁸⁹Zr-hCD103.05A. Male nude mice (BALB/cOlaHsd-Foxn1nu, Envigo, The Netherlands) were subcutaneously (sc) inoculated with CHO.K1 or CHO.CD103 (5×10⁶ in 300 μL 1:1 PBS and high growth factor Matrigel (BD Biosciences, Breda, The Netherlands)). Xenografts were allowed to grow to at least 200 mm3. For microPET imaging with mAbs, xenograft-bearing mice (n=3 per group) were injected intravenously (iv) via the penile vein with 8.7±0.48 μg ⁸⁹Zr-CD103.01A or 8.76±0.42 μg ⁸⁹Zr-CD103.05A. MicroPET scans were made 1, 3 and 6 days post injection (pi) using a Focus 220 PET scanner (CTI Siemens), followed by ex vivo biodistribution analysis after the final scan. For biodistribution experiments with Fab fragments, xenograft-bearing mice (n=2 or 3 per group) were injected intravenously (iv) via the penile vein with ~10 μg ⁸⁹Zr-Fab.hCD103.01.C1 or ⁸⁹Zr-Fab.hCD103.05.C1 followed by ex vivo biodistribution analysis after 24 hours.

Scans were reconstructed and in vivo quantification was performed using AMIDE (v1.0.4, Stanford University, Stanford, CA, USA). MicroPET data are presented as mean standardized uptake value (SUVmean). Region of interests (ROI) were drawn for tumor based upon ex vivo weight, assuming 1 g/ml tissue density. For blood pool measurements, a fixed-sized sphere was drawn in the center of the heart, for liver and spleen a fixed-sized ellipsoid ROI was drawn in representative parts of the organs. After the final scan, mice were sacrificed and organs of interest collected for biodistribution studies. Organs and standards of the injected tracer were counted in a calibrated well type LKB-1282-Compu-gamma system (LKB WALLAC) and weighed. After decay correction, ex vivo tissue activity was expressed as the percentage of injected dose per gram tissue (% ID/g).

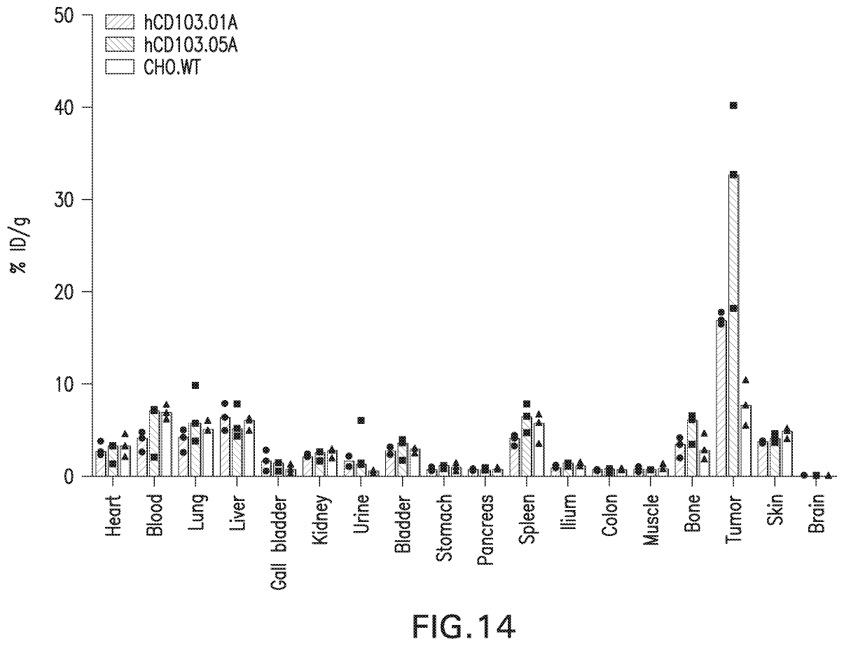

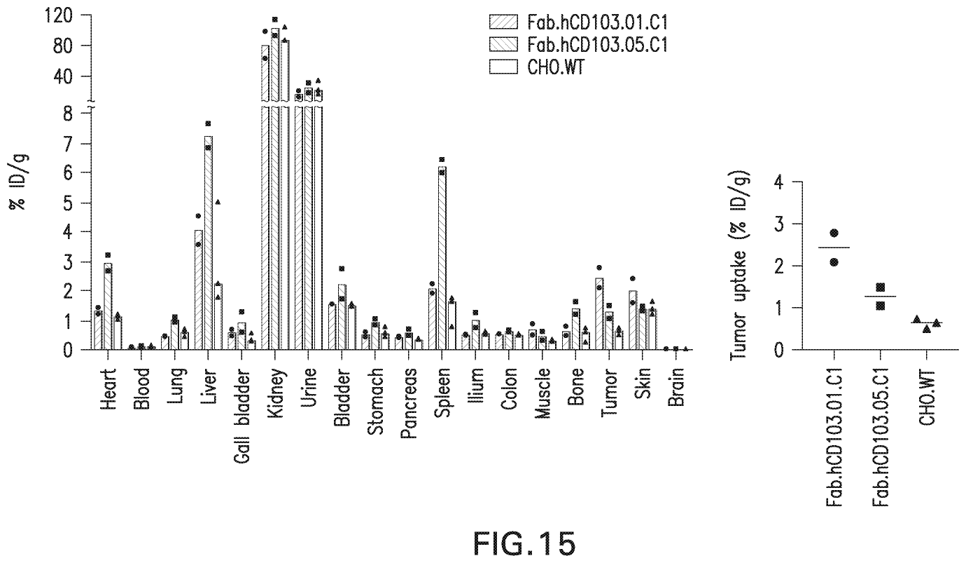

CD103 membrane expression in CHO.K1-hCD103/hBeta7 is comparable to CD103 expression in TILs (data not shown). PET scans of CHO.K1-hCD103/hBeta7 tumor bearing mice showed that ⁸⁹Zr-hCD103.01A and ⁸⁹Zr-hCD103.05A tumor uptake increased over time (FIG. 13B), with highest tumor and least background organ uptake observed at day 6 post injection (median mean standardized uptake value (SUVₘₑₐₙ) tumor: 2.7, median SUV mean blood: 0.9 for ⁸⁹Zr-hCD103.01A and median SUVₘₑₐₙ tumor: 3.0, median SUVₘₑₐₙ blood: 0.9 for ⁸⁹Zr-hCD103.05A; FIGS. 13 C, D and E). ⁸⁹Zr-hCD103.01A showed no accumulation in CHO.K1 WT xenografts (SUVₘₑₐₙ tumor: 1.5, SUVₘₑₐₙ blood: 1.4), which was used as a non-specific control group. Similarly, ex vivo biodistribution analysis on day 6 showed high CD103 specific ⁸⁹Zr-hCD103.01A and ⁸⁹Zr-hCD103.05A tumor uptake (17.0 and 32.8 percentage of injected dose per gram tissue (% ID/g), respectively, for CHO.K1-hCD103/hBeta7 vs. 7.8% ID/g for CHO.K1 WT) and no major sink organs (FIG. 14). For the Fab fragments, ex vivo biodistribution analysis after 24 hours post injection showed CD103 specific ⁸⁹Zr-Fab.hCD103.01.C1 and ⁸⁹Zr-Fab.hCD103.05.C1 tumor uptake (2.44 and 1.27% ID/g, respectively, for CHO.K1-hCD103/hBeta7 vs. 0.64% ID/g for CHO.K1 WT) (FIG. 15).

### EXAMPLE 13: USE OF A CD103 TARGETING RADIOPHARMACEUTICAL TO MONITOR TREATMENT

Prior to initiation of (immuno) therapy, a ‘CD103 PET’ scan is performed, alone or combined with a low-dose CT scan. Uptake of the CD103 radiopharmaceutical is quantified. CD103 PET-CT scans may be repeated and quantified at intervals during (immuno) therapy. Baseline uptake, on-treatment uptake, or treatment-induced changes in uptake from baseline are used to guide clinical decision making. This may include, but is not limited to, continuation of therapy, cessation of therapy or dose-adjustments.

### EXAMPLE 14: SEQUENCES

| | SEQ | |
|---|---|---|
| Description | ID NO: | SEQUENCE |
| hCD103.01A heavy | 1 | GDSITSGY |
| chain CDR1 (amino | | |
| acid sequence) | | |
| hCD103.01A heavy | 2 | ITYSGST |
| chain CDR2 (amino | | |
| acid sequence) | | |
| hCD103.01A heavy | 3 | TRHYYGSDAMDY |
| chain CDR3 (amino | | |
| acid sequence) | | |
| hCD103.01A light | 4 | QDVSIA |
| chain CDR1 (amino | | |
| acid sequence) | | |
| hCD103.01A light | 5 | SAS |
| chain CDR2 (amino | | |
| acid sequence) | | |
| hCD103.01A light | 6 | QQHYSTPWT |
| chain CDR3 (amino | | |
| acid sequence) | | |
| hCD103.01A heavy | 7 | EVQLQESGPSLVKPSQTLSLTCSVTGDSITSGYWN |
| chain (amino acid | | WIRKFPGNKLEYMGYITYSGSTYYNPSLKSRISITR |
| sequence) | | DTSKNQYYLQLNSVTTEDTATYYCTRHYYGSDA |
| | | MDYWGQGTSVTVSS |
| hCD103.01A light | 8 | DIVMTQSHKFMSTSVGDRVSITCKASQDVSIAVAW |
| chain (amino acid | | YQQRPGQSPKLLIYSASYRYTGVPDRFTGSGSGTD |
| sequence) | | FTFTISSVQAEDLAVYYCQQHYSTPWTFGGGTKLE |
| | | IK |
| hCD103.02A heavy | 9 | GDSITSGY |
| chain CDR1 (amino | | |
| acid sequence) | | |
| hCD103.02A heavy | 10 | ITYSGST |
| chain CDR2 (amino | | |
| acid sequence) | | |
| hCD103.02A heavy | 11 | ARGYYGSDAMDY |
| chain CDR3 (amino | | |
| acid sequence) | | |
| hCD103.02A light | 12 | QDVSTA |
| chain CDR1 (amino | | |
| acid sequence) | | |
| hCD103.02A light | 13 | SAS |
| chain CDR2 (amino | | |
| acid sequence) | | |
| hCD103.02A light | 14 | QQHYSPPWT |
| chain CDR3 (amino | | |
| acid sequence) | | |
| hCD103.02A heavy | 15 | EVQLQESGPSLVKPSQTLSLTCSVTGDSITSGYWN |
| chain (amino acid | | WIRKFPGNKLEYMGYITYSGSTYYNPSLKSRISITR |
| sequence) | | DTSKNQYYLQFNSVTTEDTATYYCARGYYGSDA |
| | | MDYWGQGTSVTVSS |
| hCD103.02A light | 16 | DIVMTQSHKFMSTSVGDRVSITCKASQDVSTAVA |
| chain (amino acid | | WYQQKPGQSPKLLIYSASYRYTGVPDRFTGSGSGT |
| sequence) | | DFTFTISSVQAEDLAVYYCQQHYSPPWTFGGGTKL |
| | | EIK |
| hCD103.05A heavy | 17 | GYSFTGYN |
| chain CDR1 (amino | | |
| acid sequence) | | |
| hCD103.05A heavy | 18 | IDPYYGGT |
| chain CDR2 (amino | | |
| acid sequence) | | |
| hCD103.05A heavy | 19 | ARSFYGYDAGSPYNYAMDY |
| chain CDR3 (amino | | |
| acid sequence) | | |
| hCD103.05A light | 20 | QDVGTF |
| chain CDR1 (amino | | |
| acid sequence) | | |
| hCD103.05A light | 21 | WAS |
| chain CDR2 (amino | | |
| acid sequence) | | |
| hCD103.05A light | 22 | HQYSSYPYT |
| chain CDR3 (amino | | |
| acid sequence) | | |
| hCD103.05A heavy | 23 | EVQLQQSGPELEKPGASAKISCKASGYSFTGYNMN |
| chain (amino acid | | WVKQSNGKSLEWIGNIDPYYGGTSYNQKFKGKAT |
| sequence) | | LTVDKSSSTAYMQLKSLTSEDSAVYYCARSFYGY |
| | | DAGSPYNYAMDYWGQGTPVTVSP |
| hCD103.05A light | 24 | DIVMTQSHKFMSTSVGDRVSITCKASQDVGTFVA |
| chain (amino acid | | WYQQKPGQSPKLLIYWASTRHTGVPDRFTGSGSG |
| sequence) | | TDFTLTISNVQSEDLADYFCHQYSSYPYTFGGGTQ |
| | | LEIK |
| hCD103.06A heavy | 25 | GYTFTSYW |
| chain CDR1 (amino | | |
| acid sequence) | | |
| hCD103.06A heavy | 26 | IYPGSGST |
| chain CDR2 (amino | | |
| acid sequence) | | |
| hCD103.06A heavy | 27 | TRGVYDNPYYFDY |
| chain CDR3 (amino | | |
| acid sequence) | | |
| hCD103.06A light | 28 | DHINNW |
| chain CDR1 (amino | | |
| acid sequence) | | |
| hCD103.06A light | 29 | GAT |
| chain CDR2 (amino | | |
| acid sequence) | | |
| hCD103.06A light | 30 | QQYWSIPLT |
| chain CDR3 (amino | | |
| acid sequence) | | |
| hCD103.06A heavy | 31 | QVQLQQPGSELVRPGASVKLSCKASGYTFTSYWM |
| chain (amino acid | | HWVKQRHGQGLEWIGNIYPGSGSTNYDEKFKSKG |
| sequence) | | TLTVDTSSSTAYMHLSSLTSEDSAVYYCTRGVYDN |
| | | PYYFDYWGQGTTLTVSS |
| hCD103.06A light | 32 | DIQMTQSSSNLSVSLGGRVTITCKASDHINNWLAW |
| chain (amino acid | | YQQKPGNAPRVLISGATSLETGVPSRFSGSGSGKD |
| sequence) | | YTLSITSLQTEDVATYYCQQYWSIPLTFGAGTKLE |
| | | LK |
| hCD103.07A heavy | 33 | GDSITSGY |
| chain CDR1 (amino | | |
| acid sequence) | | |
| hCD103.07A heavy | 34 | ITYSGST |
| chain CDR2 (amino | | |
| acid sequence) | | |
| hCD103.07A heavy | 35 | ARNYYGSSSMDY |
| chain CDR3 (amino | | |
| acid sequence) | | |
| hCD103.07A light | 36 | QNVGSD |
| chain CDR1 (amino | | |
| acid sequence) | | |
| hCD103.07A light | 37 | SAS |
| chain CDR2 (amino | | |
| acid sequence) | | |
| hCD103.07A light | 38 | QQYNSYPST |
| chain CDR3 (amino | | |
| acid sequence) | | |
| hCD103.07A heavy | 39 | EVQLQESGPSLVKPSQTLSLTCSVTGDSITSGYWN |
| chain (amino acid | | WIRKFPGNKLEYMGYITYSGSTYYNPSLKSRISITR |
| sequence) | | DTSKNQYYLQLNSVTTEDTATYYCARNYYGSSSM |
| | | DYWGQGTSVTVSS |
| hCD103.07A light | 40 | DIVMTQSQKFMSTSVADRVSVTCKASQNVGSDVA |
| chain (amino acid | | WYQQKPGQSPKSLIYSASYRYSGVPDRFTGSGSGT |
| sequence) | | DFTLTISNVQSEDLAEYFCQQYNSYPSTFGGGTKL |
| | | EIK |
| human integrin | 41 | MWLFHTLLCIASLALLAAFNVDVARPWLTPKGGA |
| Alpha-E (amino acid | | PFVLSSLLHQDPSTNQTWLLVTSPRTKRTPGPLHR |
| sequence) | | CSLVQDEILCHPVEHVPIPKGRHRGVTVVRSHHGV |
| | | LICIQVLVRRPHSLSSELTGTCSLLGPDLRPQAQAN |
| | | FFDLENLLDPDARVDTGDCYSNKEGGGEDDVNTA |
| | | RQRRALEKEEEEDKEEEEDEEEEEAGTEIAIILDGS |
| | | GSIDPPDFQRAKDFISNMMRNFYEKCFECNFALVQ |
| | | YGGVIQTEFDLRDSQDVMASLARVQNITQVGSVT |
| | | KTASAMQHVLDSIFTSSHGSRRKASKVMVVLTDG |
| | | GIFEDPLNLTTVINSPKMQGVERFAIGVGEEFKSAR |
| | | TARELNLIASDPDETHAFKVTNYMALDGLLSKLRY |
| | | NIISMEGTVGDALHYQLAQIGFSAQILDERQVLLG |
| | | AVGAFDWSGGALLYDTRSRRGRFLNQTAAAAAD |
| | | AEAAQYSYLGYAVAVLHKTCSLSYIAGAPRYKHH |
| | | GAVFELQKEGREASFLPVLEGEQMGSYFGSELCPV |
| | | DIDMDGSTDFLLVAAPFYHVHGEEGRVYVYRLSE |
| | | QDGSFSLARILSGHPGFTNARFGFAMAAMGDLSQ |
| | | DKLTDVAIGAPLEGFGADDGASFGSVYIYNGHWD |
| | | GLSASPSQRIRASTVAPGLQYFGMSMAGGFDISGD |
| | | GLADITVGTLGQAVVFRSRPVVRLKVSMAFTPSAL |
| | | PIGFNGVVNVRLCFEISSVTTASESGLREALLNFTL |
| | | DVDVGKQRRRLQCSDVRSCLGCLREWSSGSQLCE |
| | | DLLLMPTEGELCEEDCFSNASVKVSYQLQTPEGQT |
| | | DHPQPILDRYTEPFAIFQLPYEKACKNKLFCVAELQ |
| | | LATTVSQQELVVGLTKELTLNINLTNSGEDSYMTS |
| | | MALNYPRNLQLKRMQKPPSPNIQCDDPQPVASVLI |
| | | MNCRIGHPVLKRSSAHVSVVWQLEENAFPNRTAD |
| | | ITVTVTNSNERRSLANETHTLQFRHGFVAVLSKPSI |
| | | MYVNTGQGLSHHKEFLFHVHGENLFGAEYQLQIC |
| | | VPTKLRGLQVVAVKKLTRTQASTVCTWSQERACA |
| | | YSSVQHVEEWHSVSCVIASDKENVTVAAEISWDH |
| | | SEELLKDVTELQILGEISFNKSLYEGLNAENHRTKI |
| | | TVVFLKDEKYHSLPIIIKGSVGGLLVLIVILVILFKC |
| | | GFFKRKYQQLNLESIRKAQLKSENLLEEEN |
| human integrin Beta- | 42 | MVALPMVLVLLLVLSRGESELDAKIPSTGDATEW |
| 7 (amino acid | | RNPHLSMLGSCQPAPSCQKCILSHPSCAWCKQLNF |
| sequence) | | TASGEAEARRCARREELLARGCPLEELEEPRGQQE |
| | | VLQDQPLSQGARGEGATQLAPQRVRVTLRPGEPQ |
| | | QLQVRFLRAEGYPVDLYYLMDLSYSMKDDLERV |
| | | RQLGHALLVRLQEVTHSVRIGFGSFVDKTVLPFVS |
| | | TVPSKLRHPCPTRLERCQSPFSFHHVLSLTGDAQAF |
| | | EREVGRQSVSGNLDSPEGGFDAILQAALCQEQIGW |
| | | RNVSRLLVFTSDDTFHTAGDGKLGGIFMPSDGHCH |
| | | LDSNGLYSRSTEFDYPSVGQVAQALSAANIQPIFA |
| | | VTSAALPVYQELSKLIPKSAVGELSEDSSVVWQLIM |
| | | DAYNSLSSTVTLEHSSLPPGVHISYESQCEGPEKRE |
| | | GKAEDRGQCNHVRINQTVTFWVSLQATHCLPEPH |
| | | LLRLRALGFSEELIVELHTLCDCNCSDTQPQAPHCS |
| | | DGQGHLQCGVCSCAPGRLGRLCECSVAELSSPDLE |
| | | SGCRAPNGTGPLCSGKGHCQCGRCSCSGQSSGHL |
| | | CECDDASCERHEGILCGGFGRCQCGVCHCHANRT |
| | | GRACECSGDMDSCISPEGGLCSGHGRCKCNRCQC |
| | | LDGYYGALCDQCPGCKTPCERHRDCAECGAFRTG |
| | | PLATNCSTACAHTNVTLALAPILDDGWCKERTLD |
| | | NQLFFFLVEDDARGTVVLRVRPQEKGADHTQAIV |
| | | LGCVGGIVAVGLGLVLAYRLSVEIYDRREYSRFEK |
| | | EQQQLNWKQDSNPLYKSAITTTINPRFQEADSPTL |
| rhesus integrin | 43 | MWLVHTLLCMASLAPLAAFNVDVARPWLTPKGG |
| Alpha-E (amino acid | | APFVLSSLLHQDPGTNHTWLLVTSPRTERTPVPLH |
| sequence) | | RCSLVQDEILCHSVEHVPIPKGRHRGVTVARSHHG |
| | | VLICIQVLARRPYSLSSEFTGTCGLLGPDLRPQAQA |
| | | NFFDLENLLDPDARVDTGDCYSNKEGSRGEDVNT |
| | | ARRRRALEKEEEEEDEEEEEDEEEEAGTEIAIILDGS |
| | | GSIDPPDFQRAKDFISNMMRNFYEKCFECNFALVQ |
| | | YGGVIQTEFDLRDSQDVMASLAKVQNITQVGSVT |
| | | KTASAMQHVLDNIFTSSHGSRRKASKVMVVLTDG |
| | | GIFEDPLDLTTVINSPKMHGVERFAIGVGEEFKSAR |
| | | TERELNLIASDPDETHAFKVTNYMALDGLLSKLRY |
| | | NIISMEGTVGDALHYQLAQIGFSAQILDERQVLLG |
| | | AVGAFDWSGGALLYNTRSRRGRFLNQTAAAVDG |
| | | EAAQYSYLGYAVAVLHKTCSVSYVAGAPRYKHH |
| | | GAVFELQKEGTETSFLPVLEGEQMGSYFGSELCPV |
| | | DIDMDGTTDFLLVAAPFYHVHGEEGRVYVYRLSE |
| | | QDGSFSLARILSGHPGFASARFGFAMAAVGDISQD |
| | | KLTDVAIGAPLEGFGAGDGASFGSVYIYNGHWDG |
| | | LSAGPSQRIRASAVAPGLQYFGMSVAGGFDISGDG |
| | | LADITVGTLGRAVVFRSRPVVRLEVSMAFTPSALPI |
| | | GFNGVVNVRLCFEISSVATVSASGLRGAFLNFTLD |
| | | VDVGKERKRLQCSDGRSCLGCLREWSSGSRLCED |
| | | LLLVPTEGELHEEDCFSNATVKVGYQLQTPEGQTD |
| | | HPQPILDRYAETFAIFQLPYEKACKNKLFCVAELQL |
| | | ATTVSQQELVVGLTKELTLNISLTNSGEDSYMTSM |
| | | ALNYPRNLQFKRMQKPPSPNIQCDDPQPAASVLV |
| | | MTCRIGHPVLRRSSAHVSVVWQLEENAFPNRTADI |
| | | TVTVTNSNERRSVAEETHTLQFRHGFVAVLSKPSI |
| | | MYVHTGQVLSHHKEFVFHIHGENLFGAEYQLRIC |
| | | VPTKLRGLQIVTVKNLTRTQAFTVCTWSQERACGF |
| | | IPVQHVEEWHSVSCVIASDKENVTVAAEISVDHSE |
| | | ELLKDVTELQILGEISFNKSLYEGLNAENHRTKITV |
| | | VFLKDEKYHSLPVIIKGSIGGLLVLIVILVILFKCGFF |
| | | KRKYQQLNLENIRKAQLKSETLLEEEN |
| rhesus integrin Beta- | 44 | MGFCHVDQGMVALPVVLVLLLVLSRGESELDAKT |
| 7 (amino acid | | PSTGEATEWGNPHLSLLGSCQPAPSCQKCIVSHPSC |
| sequence) | | AWCKQLNFTASGEAEARRCARREELLARGCPLEE |
| | | LEEPRGQQEVLQDQPLSQGARGEGATQLAPQRVRI |
| | | TLRPGEPQQLQVRFLRAEGYPVDLYYLMDLSYSM |
| | | KDDLERVRQLGHALLVRLQEVTHSVRIGFGSFVD |
| | | KTVLPFVSTVPSKLRHPCPTRLERCQSPFSFHHVLS |
| | | LTGDAQAFEREVGRQSVSGNLDSPEGGFDAILQAA |
| | | LCQEQIGWRNVSRLLVFTSDDTFHTAGDGKLGGIF |
| | | MPSDGHCHLDSNGLYSRSTEFDYPSVGQVAQALS |
| | | AANIQPIFAVTSAALPVYQELSKLIPKSAVGELSED |
| | | SSNVVQLIMDAYNSLSSTVTLEHSSLPPGVHISYES |
| | | QCEGPEKTEGKAEDRGQCNHVQINQTVTFWVSLQ |
| | | ATHCLPEPHLLRLRALGFSEELIVELHTLCDCNCSD |
| | | TQAQAPHCSDGQGHLQCGVCSCAPGRLGRLCECS |
| | | EAELSSLDLESGCRAPNGTGPLCSGKGQCQCGHCS |
| | | CNGQSSGHLCECDDASCERHEGILCGGFGRCQCG |
| | | VCHCHANRTGRACECSGDMDSCISPEGGLCSGHG |
| | | RCKCNRCQCSDGYYGALCDQCPGCKTPCERHRDC |
| | | AECGAFGTGLLATNCSTACAHTNVTLVLAPILDDG |
| | | WCKERTLDNHLFFFLVEDDARGRVVLRVRPQEKG |
| | | ADHTQAIVLGCVGGIVAVGLGLVLAYRLSVEIYDR |
| | | REYSRFEKEQQQLNWKQDSNPLYKSAITTTINPRF |
| | | QEADSPIL |
| human integrin | 45 | MAWEARREPGPRRAAVRETVMLLLCLGVPTGRPY |
| Alpha-4 (amino acid | | NVDTESALLYQGPHNTLFGYSVVLHSHGANRWLL |
| sequence) | | VGAPTANWLANASVINPGAIYRCRIGKNPGQTCEQ |
| | | LQLGSPNGEPCGKTCLEERDNQWLGVTLSRQPGE |
| | | NGSIVTCGHRWKNIFYIKNENKLPTGGCYGVPPDL |
| | | RTELSKRIAPCYQDYVKKFGENFASCQAGISSFYT |
| | | KDLIVMGAPGSSYWTGSLFVYNITTNKYKAFLDK |
| | | QNQVKFGSYLGYSVGAGHFRSQHTTEVVGGAPQH |
| | | EQIGKAYIFSIDEKELNILHEMKGKKLGSYFGASVC |
| | | AVDLNADGFSDLLVGAPMQSTIREEGRVFVYINSG |
| | | SGAVMNAMETNLVGSDKYAARFGESIVNLGDIDN |
| | | DGFEDVAIGAPQEDDLQGAIYIYNGRADGISSTFSQ |
| | | RIEGLQISKSLSMFGQSISGQIDADNNGYVDVAVG |
| | | AFRSDSAVLLRTRPVVIVDASLSHPESVNRTKFDC |
| | | VENGWPSVCIDLTLCFSYKGKEVPGYIVLFYNMSL |
| | | DVNRKAESPPRFYFSSNGTSDVITGSIQVSSREANC |
| | | RTHQAFMRKDVRDILTPIQIEAAYHLGPHVISKRST |
| | | EEFPPLQPILQQKKEKDIMKKTINFARFCAHENCSA |
| | | DLQVSAKIGFLKPHENKTYLAVGSMKTLMLNVSL |
| | | FNAGDDAYETTLHVKLPVGLYFIKILELEEKQINCE |
| | | VTDNSGVVQLDCSIGYIYVDHLSRIDISFLLDVSSLS |
| | | RAEEDLSITVHATCENEEEMDNLKHSRVTVAIPLK |
| | | YEVKLTVHGFVNPTSFVYGSNDENEPETCMVEKM |
| | | NLTFHVINTGNSMAPNVSVEIMVPNSFSPQTDKLF |
| | | NILDVQTTTGECHFENYQRVCALEQQKSAMQTLK |
| | | GIVRFLSKTDKRLLYCIKADPHCLNFLCNFGKMES |
| | | GKEASVHIQLEGRPSILEMDETSALKFEIRATGFPEP |
| | | NPRVIELNKDENVAHVLLEGLHHQRPKRYFTIVIIS |
| | | SSLLLGLIVLLLISYVMWKAGFFKRQYKSILQEENR |
| | | RDSWSYINSKSNDD |

### EXAMPLE 15: REFERENCES

Khalil, D. N., Smith, E. L., Brentjens, R. J. & Wolchok, J. D. The future of cancer treatment: immunomodulation, CARs and combination immunotherapy. Nat. Rev. Clin. Oncol. 2016; 13:273-290.
Rizvi, N. A., Hellmann, M. D., Snyder, A., Kvistborg, P., Makarov, V., Havel, J. J., Lee, W., Yuan, J., Wong, P., Ho, T. S., Miller, M. L., Rekhtman, N., Moreira, A. L., Ibrahim, F., Bruggeman, C., Gasmi, B., Zappasodi, R., Maeda, Y., Sander, C., Garon, E. B., Merghoub, T., Wolchok, J. D., Schumacher, T. N. & Chan, T. A. Mutational landscape determines sensitivity to PD-1 blockade in non-small cell lung cancer. Science 2015; 348:124-128.
McGranahan, N., Furness, A. J. S., Rosenthal, R., Ramskov, S., Lyngaa, R., Saini, S. K., Jamal-Hanjani, M., Wilson, G. A., Birkbak, N. J., Hiley, C. T., Watkins, T. B. K., Shafi, S., Murugaesu, N., Mitter, R., Akarca, A. U., Linares, J., Marafioti, T., Henry, J. Y., Van Allen, E. M., Miao, D., Schilling, B., Schadendorf, D., Garraway, L. A., Makarov, V., Rizvi, N. A., Snyder, A., Hellmann, M. D., Merghoub, T., Wolchok, J. D., Shukla, S. A., Wu, C. J., Peggs, K. S., Chan, T. A., Hadrup, S. R., Quezada, S. A. & Swanton, C. Clonal neoantigens elicit T cell immunoreactivity and sensitivity to immune checkpoint blockade. Science 2016; 351:1463-1469.
Le, D. T., Durham, J. N., Smith, K. N., Wang, H., Bartlett, B. R., Aulakh, L. K., Lu, S., Kemberling, H., Wilt, C., Luber, B. S., Wong, F., Azad, N. S., Rucki, A. A., Laheru, D., Donehower, R., Zaheer, A., Fisher, G. A., Crocenzi, T. S., Lee, J. J., Greten, T. F., Duffy, A. G., Ciombor, K. K., Eyring, A. D., Lam, B. H., Joe, A., Kang, S. P., Holdhoff, M., Danilova, L., Cope, L., Meyer, C., Zhou, S., Goldberg, R. M., Armstrong, D. K., Bever, K. M., Fader, A. N., Taube, J., Housseau, F., Spetzler, D., Xiao, N., Pardoll, D. M., Papadopoulos, N., Kinzler, K. W., Eshleman, J. R., Vogelstein, B., Anders, R. A. & Diaz, L. A. Mismatch-repair deficiency predicts response of solid tumors to PD-1 blockade. Science 2017; doi: 10.1126/science.aan6733.
Sharma, P. & Allison, J. P. The future of immune checkpoint therapy. Science 2015; 348:56-61.
Adams, J. L., Smothers, J., Srinivasan, R. & Hoos, A. Big opportunities for small molecules in immuno-oncology. Nat. Rev. Drug Discov. 2015; 14:603-22.
Topalian, S. L., Drake, C. G. & Pardoll, D. M. Immune checkpoint blockade: A common denominator approach to cancer therapy. Cancer Cell 2015; 27:450-61.
Postow, M. A., Callahan, M. K. & Wolchok, J. D. Immune checkpoint blockade in cancer therapy. J. Clin. Oncol. 2015; 33:1974-82.
Buchbinder, E. & Hodi, F. S. Cytotoxic T lymphocyte antigen-4 and immune checkpoint blockade. J. Clin. Invest. 2015; 125:3377-83.
Mahoney, K. M., Rennert, P. D. & Freeman, G. J. Combination cancer immunotherapy and new immunomodulatory targets. Nat. Rev. Drug Discov. 2015; 14:561-584.
Lesterhuis, W. J., Bosco, A., Millward, M. J., Small, M., Nowak, A. K. & Lake, R. A. Dynamic versus static biomarkers in cancer immune checkpoint blockade: unravelling complexity. Nat. Rev. Drug Discov. 2017; 16:264-272.
Gnjatic, S., Bronte, V., Brunet, L. R., Butler, M. O., Disis, M. L., Galon, J., Hakansson, L. G., Hanks, B. A., Karanikas, V., Khleif, S. N., Kirkwood, J. M., Miller, L. D., Schendel, D. J., Tanneau, I., Wigginton, J. M. & Butterfield, L. H. Identifying baseline immune-related biomarkers to predict clinical outcome of immunotherapy. J. Immunother. Cancer 2017; 5:44.
Nishino, M., Ramaiya, N. H., Hatabu, H. & Hodi, F. S. Monitoring immune-checkpoint blockade: response evaluation and biomarker development. Nat. Rev. Clin. Oncol. 2017; doi: 10.1038/nrclinonc.2017.88.
Djenidi, F., Adam, J., Goubar, A., Durgeau, A., Meurice, G., de Montpréville, V., Validire, P., Besse, B. & Mami-Chouaib, F. CD8+ CD103⁺ tumor-infiltrating lymphocytes are tumor-specific tissue-resident memory T cells and a prognostic factor for survival in lung cancer patients. J. Immunol. 2015; 194:3475-3486.
Webb, J. R., Milne, K. & Nelson, B. H. PD-1 and CD103 are widely coexpressed on prognostically favorable intraepithelial CD8 T cells in human ovarian cancer. Cancer Immunol. Res. 2015; 3:926-935.
Bosmüller, H.-C., Wagner, P., Peper, J. K., Schuster, H., Pham, D. L., Greif, K., Beschorner, C., Rammensee, H.-G., Stevanović, S., Fend, F. & Staebler, A. Combined immunoscore of CD103 and CD3 identifies long-term survivors in high-grade serous ovarian cancer. Int. J. Gynecol. Cancer 2016; 26:671-679.
Workel, H. H., Komdeur, F. L., Wouters, M. C. A., Plat, A., Klip, H. G., Eggink, F. A., Wisman, G. B. A., Arts, H. J. G., Oonk, M. H. M., Mourits, M. J. E., Yigit, R., Versluis, M., Duiker, E. W., Hollema, H., de Bruyn, M. & Nijman, H. W. CD103 defines intraepithelial CD8+PD1+ tumour-infiltrating lymphocytes of prognostic significance in endometrial adenocarcinoma. Eur. J. Cancer 2016; 60:1-11.
Santoiemma, P. P., Reyes, C., Wang, L.-P., McLane, M. W., Feldman, M. D., Tanyi, J. L. & Powell, D. J. Systematic evaluation of multiple immune markers reveals prognostic factors in ovarian cancer. Gynecol. Oncol. 2016; 143:120-127.
Komdeur, F. L., Wouters, M. C. A., Workel, H. H., Tijans, A. M., Terwindt, A. L. J., Brunekreeft, K. L., Plat, A., Klip, H. G., Eggink, F. A., Leffers, N., Helfrich, W., Samplonius, D. F., Bremer, E., Wisman, G. B. A., Daemen, T., Duiker, E. W., Hollema, H., Nijman, H. W. & de Bruyn, M. CD103+ intraepithelial T cells in high-grade serous ovarian cancer are phenotypically diverse TCRαβ+CD8αβ+ T cells that can be targeted for cancer immunotherapy. Oncotarget 2016; 7:75130-75144.
Boutet, M., Gauthier, L., Leclerc, M., Gros, G., de Montpreville, V., Théret, N., Donnadieu, E. & Mami-Chouaib, F. TGFβ signaling intersects with CD103 integrin signaling to promote T-lymphocyte accumulation and antitumor activity in the lung tumor microenvironment. Cancer Res. 2016; 76:1757-1769.
Ganesan, A.-P., Clarke, J., Wood, O., Garrido-Martin, E. M., Chee, S. J., Mellows, T., Samaniego-Castruita, D., Singh, D., Seumois, G., Alzetani, A., Woo, E., Friedmann, P. S., King, E. V, Thomas, G. J., Sanchez-Elsner, T., Vijayanand, P. & Ottensmeier, C. H. Tissue-resident memory features are linked to the magnitude of cytotoxic T cell responses in human lung cancer. Nat. Immunol. 2017; 18:940-950.
Wang, B., Wu, S., Zeng, H., Liu, Z., Dong, W., He, W., Chen, X., Dong, X., Zheng, L., Lin, T. & Huang, J. CD103+ tumor infiltrating lymphocytes predict a favorable prognosis in urothelial cell carcinoma of the bladder. J. Urol. 2015; 194:556-562.
Wang, Z. Q., Milne, K., Derocher, H., Webb, J. R., Nelson, B. H. & Watson, P. H. CD103 and intratumoral immune response in breast cancer. Clin. Cancer Res. 2016; 22:6290-6297.
Kato R., Yamasaki M., Urakawa S., Nishida K., Makino T., Morimoto-Okazawa A., Kawashima A., Iwahori K., Suzuki S., Ueda R., Mori M., Satoh T., Doki Y., Wada H. Increased Tim-3+ T cells in PBMCs during nivolumab therapy correlate with responses and prognosis of advanced esophageal squamous cell carcinoma patients. Cancer Immunol Immunother. 2018. doi: 10.1007/s00262-018-2225-x.
Rocha P., Hardy-Werbin M., Naranjo D., Taus Á., Rodrigo M., Zuccarino F., Roth R., Wood O., Ottensmeier C. H., Arriola E. CD103+ CD8+ Lymphocytes Characterize the Immune Infiltration in a Case With Pseudoprogression in Squamous NSCLC. J Thorac Oncol. 2018; 13): e193-e196.
Edwards J., Wilmott J. S., Madore J., Gide T. N., Quek C., Tasker A., Ferguson A., Chen J., Hewavisenti R., Hersey P., Gebhardt T., Weninger W., Britton W. J., Saw R. P. M., Thompson J. F., Menzies A. M., Long G. V., Scolyer R. A., Palendira U. CD103+ Tumor-Resident CD8+ T Cells Are Associated with Improved Survival in Immunotherapy-Naïve Melanoma Patients and Expand Significantly During Anti-PD-1 Treatment. Clin Cancer Res. 2018; 24:3036-3045van Kruchten, M., de Vries, E. G. E., Brown, M., de Vries, E. F. J., Glaudemans, A. W. J. M., Dierckx, R. A. J. O., Schroder, C. P. & Hospers, G. A. P. PET imaging of oestrogen receptors in patients with breast cancer. Lancet Oncol. 2013; 14: e465-75.
van Kruchten, M., Hospers, G. A. P., Glaudemans, A. W. J. M., Hollema, H., Arts, H. J. G. & Reyners, A. K. L. Positron emission tomography imaging of oestrogen receptor-expression in endometrial stromal sarcoma supports oestrogen receptor-targeted therapy: case report and review of the literature. Eur. J. Cancer 2013; 49:3850-5.
Talbot, J. N., Gligorov, J., Nataf, V., Montravers, F., Huchet, V., Michaud, L., Ohnona, J., Balogova, S., Cussenot, O., Daraï, E., Lotz, J. P. & Kerrou, K. Current applications of PET imaging of sex hormone receptors with a fluorinated analogue of estradiol or of testosterone. Q. J. Nucl. Med. Mol. Imaging 2015; 59:4-17.
Grootjans, W., de Geus-Oei, L.-F., Troost, E. G. C., Visser, E. P., Oyen, W. J. G. & Bussink, J. PET in the management of locally advanced and metastatic NSCLC. Nat. Rev. Clin. Oncol. 2015; 12:395-407.
Houshmand, S., Boursi, B., Salavati, A., Simone, C. B. & Alavi, A. Applications of Fluorodeoxyglucose PET/Computed tomography in the assessment and prediction of radiation therapy-related complications. PET Clin. 2015; 10:555-71.
Bernard-Gauthier, V., Bailey, J. J., Berke, S. & Schirrmacher, R. Recent advances in the development and application of radiolabeled kinase inhibitors for PET imaging. Molecules 2015; 20:22000-27.
Challapalli, A. & Aboagye, E. O. Positron emission tomography imaging of tumor cell metabolism and application to therapy response monitoring. Front. Oncol. 2016; 6:44.
Spick, C., Herrmann, K. & Czernin, J. 18F-FDG PET/CT and PET/MRI perform equally well in vancer: Evidence from studies on more than 2,300 patients. J. Nucl. Med. 2016; 57:420-30.
Bollineni, V. R., Kramer, G. M., Jansma, E. P., Liu, Y. & Oyen, W. J. G. A systematic review on [(18) F]FLT-PET uptake as a measure of treatment response in cancer patients. Eur. J. Cancer 2016; 55:81-97.
Eggink, F. A., Van Gool, I. C., Leary, A., Pollock, P. M., Crosbie, E. J., Mileshkin, L., Jordanova, E. S., Adam, J., Freeman-Mills, L., Church, D. N., Creutzberg, C. L., De Bruyn, M., Nijman, H. W. & Bosse, T. Immunological profiling of molecularly classified high-risk endometrial cancers identifies POLE-mutant and microsatellite unstable carcinomas as candidates for checkpoint inhibition. Oncoimmunology 2017; 6: e1264565.
F. A. Ran, P. D. Hsu, J. Wright, V. Agarwala, D. A. Scott, F. Zhang, Genome engineering using the CRISPR-Cas9 system, Nat. Protoc. 8, 2281-2308 (2013).
A. Kol, A. T. van Scheltinga, M. Pool, C. Gerdes, E. de Vries, S. de Jong, ADCC responses and blocking of EGFR-mediated signaling and cell growth by combining the anti-EGFR antibodies imgatuzumab and cetuximab in NSCLC cells., Oncotarget 8, 45432-45446 (2017).
B. J. C. Quah, H. S. Warren, C. R. Parish, Monitoring lymphocyte proliferation in vitro and in vivo with the intracellular fluorescent dye carboxyfluorescein diacetate succinimidyl ester., Nat. Protoc. 2, 2049-2056 (2007).
E. C. F. Dijkers, J. G. W. Kosterink, A. P. Rademaker, L. R. Perk, G. a M. S. van Dongen, J. Bart, J. R. de Jong, E. G. E. de Vries, M. N. Lub-de Hooge, Development and characterization of clinical-grade ⁸⁹Zr-trastuzumab for HER2/neu immunoPET imaging., J. Nucl. Med. 50, 974-981 (2009).
F. A. Ran, P. D. Hsu, J. Wright, V. Agarwala, D. A. Scott, F. Zhang. Genome engineering using the CRISPR-Cas9 system. Nat. Protoc. 8:2281-2308 (2013)
P. G. Steenbakkers, F. van Meel, W. Olijve. A new approach to the generation of human or murine antibody producing hybridomas. J Immunol Methods. 152:69-77 (1992).

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57 (b) (1), to relate to each and every individual publication, database entry (e.g. Genbank sequences or GeneID entries), patent application, or patent, each of which is clearly identified in compliance with 37 C.F.R. § 1.57(b) (2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. To the extent that the references provide a definition for a claimed term that conflicts with the definitions provided in the instant specification, the definitions provided in the instant specification shall be used to interpret the claimed invention.

While the invention has been described and exemplified in sufficient detail for those skilled in this art to make and use it, various alternatives, modifications, and improvements should be apparent without departing from the spirit and scope of the invention. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patent applications, patents, publications and other references mentioned in the specification are indicative of the levels of those of ordinary skill in the art to which the invention pertains and are each incorporated herein by reference. The references cited herein are not admitted to be prior art to the claimed invention.

Unless otherwise defined, all technical and scientific terms used herein have the same meaning as commonly understood by one of ordinary skill in the art to which this invention belongs. In the case of conflict, the present specification, including definitions, will control.

The use of the articles “a”, “an”, and “the” in both the description and claims are to be construed to cover both the singular and the plural, unless otherwise indicated herein or clearly contradicted by context. The terms “comprising”, “having”, “being of” as in “being of a chemical formula”, “including”, and “containing” are to be construed as open terms (i.e., meaning “including but not limited to”) unless otherwise noted. Additionally whenever “comprising” or another open-ended term is used in an embodiment, it is to be understood that the same embodiment can be more narrowly claimed using the intermediate term “consisting essentially of” or the closed term “consisting of”.

The term “about”, “approximately”, or “approximate”, when used in connection with a numerical value, means that a collection or range of values is included. For example, “about X” includes a range of values that are ±20%, ±10%, ±5%, ±2%, ±1%, ±0.5%, ±0.2%, or ±0.1% of X, where X is a numerical value. In one embodiment, the term “about” refers to a range of values which are 10% more or less than the specified value. In another embodiment, the term “about” refers to a range of values which are 5% more or less than the specified value. In another embodiment, the term “about” refers to a range of values which are 1% more or less than the specified value.

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. For example, the terms “between X and Y” and “range from X to Y, are inclusive of X and Y and the integers there between. On the other hand, when a series of individual values are referred to in the disclosure, any range including any of the two individual values as the two end points is also conceived in this disclosure. For example, the expression “a dose of about 100 mg, 200 mg, or 400 mg” can also mean “a dose ranging from 100 to 200 mg”, “a dose ranging from 200 to 400 mg”, or “a dose ranging from 100 to 400 mg”.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. An antibody or antigen binding fragment thereof that binds to human CD103, wherein the antibody or antigen binding fragment comprises one or more, and optionally each, of:
a. a heavy chain variable region CDR1 comprising the amino acid sequence of SEQ ID NO: 9 or an amino acid sequence differing from SEQ ID NO: 9 by 1, 2, or 3 conservative substitutions,
b. a heavy chain variable region CDR2 comprising the amino acid sequence of SEQ ID NO: 10 or an amino acid sequence differing from SEQ ID NO: 10 by 1, 2, or 3 conservative substitutions,
c. a heavy chain variable region CDR3 comprising the amino acid sequence of SEQ ID NO: 11 or an amino acid sequence differing from SEQ ID NO: 11 by 1, 2, or 3 conservative substitutions,
d. a light chain variable region CDR1 comprising the amino acid sequence of SEQ ID NO: 12 or an amino acid sequence differing from SEQ ID NO: 12 by 1, 2, or 3 conservative substitutions,
e. a light chain variable region CDR2 comprising the amino acid sequence of SEQ ID NO: 13 or an amino acid sequence differing from SEQ ID NO: 13 by 1, 2, or 3 conservative substitutions, and
f. a light chain variable region CDR3 comprising the amino acid sequence of SEQ ID NO: 14 or an amino acid sequence differing from SEQ ID NO: 14 by 1, 2, or 3 conservative substitutions
or
an antibody or antigen binding fragment thereof that binds to human CD103, wherein the antibody or antigen binding fragment comprises one or more, and optionally each, of:
g. a heavy chain variable region CDR1 comprising the amino acid sequence of SEQ ID NO: 17 or an amino acid sequence differing from SEQ ID NO: 17 by 1, 2, or 3 conservative substitutions,
h. a heavy chain variable region CDR2 comprising the amino acid sequence of SEQ ID NO: 18 or an amino acid sequence differing from SEQ ID NO: 18 by 1, 2, or 3 conservative substitutions,
i. a heavy chain variable region CDR3 comprising the amino acid sequence of SEQ ID NO: 19 or an amino acid sequence differing from SEQ ID NO: 19 by 1, 2, or 3 conservative substitutions,
j. a light chain variable region CDR1 comprising the amino acid sequence of SEQ ID NO: 20 or an amino acid sequence differing from SEQ ID NO: 20 by 1, 2, or 3 conservative substitutions,
k. a light chain variable region CDR2 comprising the amino acid sequence of SEQ ID NO: 21 or an amino acid sequence differing from SEQ ID NO: 21 by 1, 2, or 3 conservative substitutions, and
l. a light chain variable region CDR3 comprising the amino acid sequence of SEQ ID NO: 22 or an amino acid sequence differing from SEQ ID NO: 22 by 1, 2, or 3 conservative substitutions
or
an antibody or antigen binding fragment thereof that binds to human CD103, wherein the antibody or antigen binding fragment comprises one or more, and optionally each, of:
m. a heavy chain variable region CDR1 comprising the amino acid sequence of SEQ ID NO: 25 or an amino acid sequence differing from SEQ ID NO: 25 by 1, 2, or 3
n. a heavy chain variable region CDR2 comprising the amino acid sequence of SEQ ID NO: 26 or an amino acid sequence differing from SEQ ID NO: 26 by 1, 2, or 3 conservative substitutions,
o. a heavy chain variable region CDR3 comprising the amino acid sequence of SEQ ID NO: 27 or an amino acid sequence differing from SEQ ID NO: 27 by 1, 2, or 3 conservative substitutions,
p. a light chain variable region CDR1 comprising the amino acid sequence of SEQ ID NO: 28 or an amino acid sequence differing from SEQ ID NO: 28 by 1, 2, or 3 conservative substitutions,
q. a light chain variable region CDR2 comprising the amino acid sequence of SEQ ID NO: 29 or an amino acid sequence differing from SEQ ID NO: 29 by 1, 2, or 3 conservative substitutions, and
r. a light chain variable region CDR3 comprising the amino acid sequence of SEQ ID NO: 30 or an amino acid sequence differing from SEQ ID NO: 30 by 1, 2, or 3 conservative substitutions or
an antibody or antigen binding fragment thereof that binds to human CD103, wherein the antibody or antigen binding fragment comprises one or more, and optionally each, of:
s. a heavy chain variable region CDR1 comprising the amino acid sequence of SEQ ID NO: 33 or an amino acid sequence differing from SEQ ID NO: 33 by 1, 2, or 3 conservative substitutions,
t. a heavy chain variable region CDR2 comprising the amino acid sequence of SEQ ID NO: 34 or an amino acid sequence differing from SEQ ID NO: 34 by 1, 2, or 3
u. a heavy chain variable region CDR3 comprising the amino acid sequence of SEQ ID NO: 35 or an amino acid sequence differing from SEQ ID NO: 35 by 1, 2, or 3 conservative substitutions,
v. a light chain variable region CDR1 comprising the amino acid sequence of SEQ ID NO: 36 or an amino acid sequence differing from SEQ ID NO: 36 by 1, 2, or 3 conservative substitutions,
w. a light chain variable region CDR2 comprising the amino acid sequence of SEQ ID NO: 37 or an amino acid sequence differing from SEQ ID NO: 37 by 1, 2, or 3 conservative substitutions, and
x. a light chain variable region CDR3 comprising the amino acid sequence of SEQ ID NO: 38 or an amino acid sequence differing from SEQ ID NO: 38 by 1, 2, or 3 conservative substitutions.

2. An antibody or antigen binding fragment thereof that binds to human CD103 according to claim 1, wherein
the heavy chain of the antibody or antigen binding fragment comprises the amino acid sequence of SEQ ID NO: 23 and the light chain of the antibody or antigen binding fragment comprises the amino acid sequence of SEQ ID NO: 24
or
the heavy chain of the antibody or antigen binding fragment comprises the amino acid sequence of SEQ ID NO: 31 and the light chain of the antibody or antigen binding fragment comprises the amino acid sequence of SEQ ID NO: 32
or
the heavy chain of the antibody or antigen binding fragment comprises the amino acid sequence of SEQ ID NO: 39 and the light chain of the antibody or antigen binding fragment comprises the amino acid sequence of SEQ ID NO: 40.

3. The antibody or antigen binding fragment of claim 1, wherein the antibody is an intact IgG, an scFv, an Fab or an F(ab′)₂.

4. The antibody or antigen binding fragment of claim 1, wherein the antibody comprises a wild-type or mutated IgG2 Fc region.

5. The antibody or antigen binding fragment of claim 1, wherein the antibody comprises a mutated IgG1 Fc region.

6. The antibody or antigen binding fragment of claim 1, wherein the antibody comprises a mutated IgG4 Fc region.

7. An antibody or antigen binding fragment thereof that binds to the same epitope of human CD103 as an antibody as an antibody according to claim 1.

8. The antibody or antigen binding fragment of claim 1, wherein the antibody or antigen binding fragment is humanized.

9. One or more nucleic acids encoding an antibody according to claim 1.

10. An expression system comprising one or more nucleic acids encoding an antibody according to claim 9 and regulatory sequences operably connected thereto configured to express the antibody in a host cell.

11. A host cell comprising expression system of claim 10.

12. The host cell of claim 11, which is a bacterial cell, a human cell, a mammalian cell, a Pichia cell, a plant cell, an HEK293 cell, or a Chinese hamster ovary cell.

13. A composition comprising the antibody or antigen binding fragment of claim 1 and a pharmaceutically acceptable carrier or diluent.

14. A method of producing an antibody or antigen binding fragment comprising:
culturing a host cell comprising a polynucleotide encoding the heavy chain and/or the light chain of the antibody or antigen binding fragment of claim 1 under conditions favorable to expression of the polynucleotide; and optionally, recovering the antibody or antigen binding fragment from the host cell and/or culture medium.

15. A method for detecting the presence of CD103 in a biological specimen comprising contacting the specimen with an anti-CD103 antibody of claim 1 or antigen binding fragment thereof; and
detecting the presence or amount of binding of the antibody or antigen binding fragment in a complex with CD103 present in the biological specimen, wherein detection of the complex indicates the presence or amount of CD103 present in the biological specimen.

16. A method according to claim 15, wherein the antibody or antigen binding fragment comprises a diagnostic label.

17. A method according to claim 15, wherein the detecting step comprises performing PET imaging, single-photon emission computed tomography (SPECT) imaging, MRI, optical imaging, or (photo) acoustic imaging.

18. A method according to claim 16, wherein the diagnostic label is selected from the group consisting of ¹¹C, ¹³N, ¹⁵O, ^{99m}Tc, ⁶¹Cu, ⁶²Cu, ⁶⁴Cu, ⁶⁷Cu, ¹⁸F, ¹⁹F, ⁶⁶Ga, ⁶⁷Ga, ⁶⁸Ga, ⁷²Ga, ¹²³I, ¹²⁴I, ¹¹¹In, ¹⁷⁷Lu, ⁴⁴Sc, ⁴⁷Sc, ⁸⁶Y, ⁸⁸Y, ⁹⁰Y, ⁴⁵Ti, ⁸⁹Zr, indocyanine green, IRDye 800CW, fluorescein (FITC), and a magnetic (e.g., iron oxide) nanoparticle.

19. A method according to claim 15, wherein the antibody or antigen binding fragment does not block CD103 binding to E-cadherin.

20. A method according to claim 15, wherein the antibody or antigen binding fragment at least partially blocks CD103 binding to E-cadherin.

21. A method according to claim 15, wherein the detecting step comprises an in vivo imaging method for detecting the complex.

22. A method according to claim 21, wherein the detecting step comprises an in vivo imaging method for detecting the complex in a tumor.

23. An imaging agent, comprising:
A detectably labeled anti-CD103 antibody or antigen binding fragment thereof of claim 1.

24. An imaging agent according to claim 23, wherein the antibody is an anti-CD103 antibody of claim 1 or antigen binding fragment thereof that is detectably labeled by a label selected from the group consisting of ¹¹C, ¹³N, ¹⁵O, ^{99m}Tc, ⁶¹Cu, ⁶²Cu, ⁶⁴Cu, ⁶⁷Cu, ¹⁸F, ¹⁹F, ⁶⁶Ga, ⁶⁷Ga, ⁶⁸Ga, ⁷²Ga, ¹²³I, ¹²⁴I, ¹¹¹In, ¹⁷⁷Lu, ⁴⁴Sc, ⁴⁷Sc, ⁸⁶Y, ⁸⁸Y, ⁹⁰Y, ⁴⁵Ti, ⁸⁹Zr, indocyanine green, IRDye 800CW, fluorescein (FITC), and a magnetic (e.g., iron oxide) nanoparticle.

25. A method for treating or preventing a CD103 signaling-mediated condition in an individual in need thereof, comprising:
administering an effective amount of an anti-CD103 antibody of claim 1 or antigen binding fragment thereof to the individual wherein the anti-CD103 antibody is optionally coupled to a cytotoxic agent.

26. A method for inhibiting CD103 signaling in a cell, comprising:
contacting the cell with an anti-CD103 antibody of claim 1 or antigen binding fragment thereof.

27. A method for inhibiting CD103 binding to E-cadherin present on a cell, comprising:
contacting the cell with an anti-CD103 antibody of claim 1 or antigen binding fragment thereof.

28. A method for depleting CD103-expressing cells in an individual, comprising:
administering an effective amount of an anti-CD103 antibody of claim 1 or antigen binding fragment thereof to the individual, wherein the anti-CD103 antibody is optionally coupled to a cytotoxic agent.

29. A method for treating or preventing a disease selected from the group consisting of Hairy Cell leukemia, HCLv, intestinal and extraintestinal lymphomas, enteropathy-associated T-cell lymphoma (EATL), T-lymphoblastic leukemia/lymphoma (T-ALL), T-cell prolymphocytic leukemia (T-PLL), adult T cell leukemia/lymphoma (ATLL), mycosis fungoides (MF), anaplastic large cell lymphoma ALCL, cutaneous T-cell lymphoma (CTCL), Sezary Syndrome (SS), Alzheimer's disease, Parkinson's disease, multiple sclerosis, IgM polyneuropathies, myasthenia gravis, atopic dermatitis, allergy, asthma, systemic inflammatory response syndrome (SIRS), sepsis, septic shock, atherosclerosis, celiac disease, dermatomyositis, scleroderma, interstitial cystitis, transplant rejection, graft-versus-host disease, Aicardi-Goutieres Syndrome, Hutchison Guilford progeria syndrome, Singleton-Merten Syndrome, proteasome-associated autoinflammatory syndrome, SAVI (STING-associated vasculopathy with onset in infancy), CANDLE (Chronic Atypical Neutrophilic Dermatosis with Lipodystrophy and Elevated Temperature) syndrome, chilblain lupus erythematosus, systemic lupus erythematosus, rheumatoid arthritis, juvenile rheumatoid arthritis, Wegener's disease, inflammatory bowel disease (e.g. ulcerative colitis, Crohn's disease), idiopathic thrombocytopeni purpura, thrombotic thrombocytopenia purpura, autoimmune thrombocytopenia, multiple sclerosis, psoriasis, IgA nephropathy, IgM polyneuropathies, glomerulonephritis, autoimmune myocarditis, myasthenia gravis, vasculitis, Type 1 diabetes, Type 2 diabetes, Sjorgen's syndrome, X-linked reticulate pigmentary disorder, polymyositis, spondyloenchondrodysplasia, and age-related macular degeneration in an individual in need thereof, comprising:
administering an effective amount of an anti-CD103 antibody of claim 1 or antigen binding fragment thereof to the individual, wherein the anti-CD103 antibody is optionally coupled to a cytotoxic agent.


SEQUENCE LISTING (45 sequences)

SEQ ID NO: 1 - protein, 8 aa, synthetic construct
  REGION 1..8: note = Synthetic: hCD103.01A heavy chain CDR1 (amino acid sequence)
GDSITSGY

SEQ ID NO: 2 - protein, 7 aa, synthetic construct
  REGION 1..7: note = Synthetic: hCD103.01A heavy chain CDR2 (amino acid sequence)
ITYSGST

SEQ ID NO: 3 - protein, 12 aa, synthetic construct
  REGION 1..12: note = Synthetic: hCD103.01A heavy chain CDR3 (amino acid sequence)
TRHYYGSDAMDY

SEQ ID NO: 4 - protein, 6 aa, synthetic construct
  REGION 1..6: note = Synthetic: hCD103.01A light chain CDR1 (amino acid sequence)
QDVSIA

SEQ ID NO: 5 - skipped

SEQ ID NO: 6 - protein, 9 aa, synthetic construct
  REGION 1..9: note = Synthetic: hCD103.01A light chain CDR3 (amino acid sequence)
QQHYSTPWT

SEQ ID NO: 7 - protein, 118 aa, synthetic construct
  REGION 1..118: note = Synthetic: hCD103.01A heavy chain (amino acid sequence)
EVQLQESGPSLVKPSQTLSLTCSVTGDSITSGYWNWIRKFPGNKLEYMGYITYSGSTYYNPSLKSRISITRDTSKNQYYLQLNSVTTEDTATYYCTRHYYGSDAMDYWGQGTSVTVSS

SEQ ID NO: 8 - protein, 107 aa, synthetic construct
  REGION 1..107: note = Synthetic: hCD103.01A light chain (amino acid sequence)
DIVMTQSHKFMSTSVGDRVSITCKASQDVSIAVAWYQQRPGQSPKLLIYSASYRYTGVPDRFTGSGSGTDFTFTISSVQAEDLAVYYCQQHYSTPWTFGGGTKLEIK

SEQ ID NO: 9 - protein, 8 aa, synthetic construct
  REGION 1..8: note = Synthetic: hCD103.02A heavy chain CDR1 (amino acid sequence)
GDSITSGY

SEQ ID NO: 10 - protein, 7 aa, synthetic construct
  REGION 1..7: note = Synthetic: hCD103.02A heavy chain CDR2 (amino acid sequence)
ITYSGST

SEQ ID NO: 11 - protein, 12 aa, synthetic construct
  REGION 1..12: note = Synthetic: hCD103.02A heavy chain CDR3 (amino acid sequence)
ARGYYGSDAMDY

SEQ ID NO: 12 - protein, 6 aa, synthetic construct
  REGION 1..6: note = Synthetic: hCD103.02A light chain CDR1 (amino acid sequence)
QDVSTA

SEQ ID NO: 13 - skipped

SEQ ID NO: 14 - protein, 9 aa, synthetic construct
  REGION 1..9: note = Synthetic: hCD103.02A light chain CDR3 (amino acid sequence)
QQHYSPPWT

SEQ ID NO: 15 - protein, 118 aa, synthetic construct
  REGION 1..118: note = Synthetic: hCD103.02A heavy chain (amino acid sequence)
EVQLQESGPSLVKPSQTLSLTCSVTGDSITSGYWNWIRKFPGNKLEYMGYITYSGSTYYNPSLKSRISITRDTSKNQYYLQFNSVTTEDTATYYCARGYYGSDAMDYWGQGTSVTVSS

SEQ ID NO: 16 - protein, 107 aa, synthetic construct
  REGION 1..107: note = Synthetic: hCD103.02A light chain (amino acid sequence)
DIVMTQSHKFMSTSVGDRVSITCKASQDVSTAVAWYQQKPGQSPKLLIYSASYRYTGVPDRFTGSGSGTDFTFTISSVQAEDLAVYYCQQHYSPPWTFGGGTKLEIK

SEQ ID NO: 17 - protein, 8 aa, synthetic construct
  REGION 1..8: note = Synthetic: hCD103.05A heavy chain CDR1 (amino acid sequence)
GYSFTGYN

SEQ ID NO: 18 - protein, 8 aa, synthetic construct
  REGION 1..8: note = Synthetic: hCD103.05A heavy chain CDR2 (amino acid sequence)
IDPYYGGT

SEQ ID NO: 19 - protein, 19 aa, synthetic construct
  REGION 1..19: note = Synthetic: hCD103.05A heavy chain CDR3 (amino acid sequence)
ARSFYGYDAGSPYNYAMDY

SEQ ID NO: 20 - protein, 6 aa, synthetic construct
  REGION 1..6: note = Synthetic: hCD103.05A light chain CDR1 (amino acid sequence)
QDVGTF

SEQ ID NO: 21 - skipped

SEQ ID NO: 22 - protein, 9 aa, synthetic construct
  REGION 1..9: note = Synthetic: hCD103.05A light chain CDR3 (amino acid sequence)
HQYSSYPYT

SEQ ID NO: 23 - protein, 126 aa, synthetic construct
  REGION 1..126: note = Synthetic: hCD103.05A heavy chain (amino acid sequence)
EVQLQQSGPELEKPGASAKISCKASGYSFTGYNMNWVKQSNGKSLEWIGNIDPYYGGTSYNQKFKGKATLTVDKSSSTAYMQLKSLTSEDSAVYYCARSFYGYDAGSPYNYAMDYWGQGTPVTVSP

SEQ ID NO: 24 - protein, 107 aa, synthetic construct
  REGION 1..107: note = Synthetic: hCD103.05A light chain (amino acid sequence)
DIVMTQSHKFMSTSVGDRVSITCKASQDVGTFVAWYQQKPGQSPKLLIYWASTRHTGVPDRFTGSGSGTDFTLTISNVQSEDLADYFCHQYSSYPYTFGGGTQLEIK

SEQ ID NO: 25 - protein, 8 aa, synthetic construct
  REGION 1..8: note = Synthetic: hCD103.06A heavy chain CDR1 (amino acid sequence)
GYTFTSYW

SEQ ID NO: 26 - protein, 8 aa, synthetic construct
  REGION 1..8: note = Synthetic: hCD103.06A heavy chain CDR2 (amino acid sequence)
IYPGSGST

SEQ ID NO: 27 - protein, 13 aa, synthetic construct
  REGION 1..13: note = Synthetic: hCD103.06A heavy chain CDR3 (amino acid sequence)
TRGVYDNPYYFDY

SEQ ID NO: 28 - protein, 6 aa, synthetic construct
  REGION 1..6: note = Synthetic: hCD103.06A light chain CDR1 (amino acid sequence)
DHINNW

SEQ ID NO: 29 - skipped

SEQ ID NO: 30 - protein, 9 aa, synthetic construct
  REGION 1..9: note = Synthetic: hCD103.06A light chain CDR3 (amino acid sequence)
QQYWSIPLT

SEQ ID NO: 31 - protein, 120 aa, synthetic construct
  REGION 1..120: note = Synthetic: hCD103.06A heavy chain (amino acid sequence)
QVQLQQPGSELVRPGASVKLSCKASGYTFTSYWMHWVKQRHGQGLEWIGNIYPGSGSTNYDEKFKSKGTLTVDTSSSTAYMHLSSLTSEDSAVYYCTRGVYDNPYYFDYWGQGTTLTVSS

SEQ ID NO: 32 - protein, 107 aa, synthetic construct
  REGION 1..107: note = Synthetic: hCD103.06A light chain (amino acid sequence)
DIQMTQSSSNLSVSLGGRVTITCKASDHINNWLAWYQQKPGNAPRVLISGATSLETGVPSRFSGSGSGKDYTLSITSLQTEDVATYYCQQYWSIPLTFGAGTKLELK

SEQ ID NO: 33 - protein, 8 aa, synthetic construct
  REGION 1..8: note = Synthetic: hCD103.07A heavy chain CDR1 (amino acid sequence)
GDSITSGY

SEQ ID NO: 34 - protein, 7 aa, synthetic construct
  REGION 1..7: note = Synthetic: hCD103.07A heavy chain CDR2 (amino acid sequence)
ITYSGST

SEQ ID NO: 35 - protein, 12 aa, synthetic construct
  REGION 1..12: note = Synthetic: hCD103.07A heavy chain CDR3 (amino acid sequence)
ARNYYGSSSMDY

SEQ ID NO: 36 - protein, 6 aa, synthetic construct
  REGION 1..6: note = Synthetic: hCD103.07A light chain CDR1 (amino acid sequence)
QNVGSD

SEQ ID NO: 37 - skipped

SEQ ID NO: 38 - protein, 9 aa, synthetic construct
  REGION 1..9: note = Synthetic: hCD103.07A light chain CDR3 (amino acid sequence)
QQYNSYPST

SEQ ID NO: 39 - protein, 118 aa, synthetic construct
  REGION 1..118: note = Synthetic: hCD103.07A heavy chain (amino acid sequence)
EVQLQESGPSLVKPSQTLSLTCSVTGDSITSGYWNWIRKFPGNKLEYMGYITYSGSTYYNPSLKSRISITRDTSKNQYYLQLNSVTTEDTATYYCARNYYGSSSMDYWGQGTSVTVSS

SEQ ID NO: 40 - protein, 107 aa, synthetic construct
  REGION 1..107: note = Synthetic: hCD103.07A light chain (amino acid sequence)
DIVMTQSQKFMSTSVADRVSVTCKASQNVGSDVAWYQQKPGQSPKSLIYSASYRYSGVPDRFTGSGSGTDFTLTISNVQSEDLAEYFCQQYNSYPSTFGGGTKLEIK

SEQ ID NO: 41 - protein, 1179 aa, Homo sapiens
  REGION 1..1179: note = human integrin Alpha-E (amino acid sequence)
MWLFHTLLCIASLALLAAFNVDVARPWLTPKGGAPFVLSSLLHQDPSTNQTWLLVTSPRTKRTPGPLHRCSLVQDEILCHPVEHVPIPKGRHRGVTVVRSHHGVLICIQVLVRRPHSLSSELTGTCSLLGPDLRPQAQANFFDLENLLDPDARVDTGDCYSNKEGGGEDDVNTARQRRALEKEEEEDKEEEEDEEEEEAGTEIAIILDGSGSIDPPDFQRAKDFISNMMRNFYEKCFECNFALVQYGGVIQTEFDLRDSQDVMASLARVQNITQVGSVTKTASAMQHVLDSIFTSSHGSRRKASKVMVVLTDGGIFEDPLNLTTVINSPKMQGVERFAIGVGEEFKSARTARELNLIASDPDETHAFKVTNYMALDGLLSKLRYNIISMEGTVGDALHYQLAQIGFSAQILDERQVLLGAVGAFDWSGGALLYDTRSRRGRFLNQTAAAAADAEAAQYSYLGYAVAVLHKTCSLSYIAGAPRYKHHGAVFELQKEGREASFLPVLEGEQMGSYFGSELCPVDIDMDGSTDFLLVAAPFYHVHGEEGRVYVYRLSEQDGSFSLARILSGHPGFTNARFGFAMAAMGDLSQDKLTDVAIGAPLEGFGADDGASFGSVYIYNGHWDGLSASPSQRIRASTVAPGLQYFGMSMAGGFDISGDGLADITVGTLGQAVVFRSRPVVRLKVSMAFTPSALPIGFNGVVNVRLCFEISSVTTASESGLREALLNFTLDVDVGKQRRRLQCSDVRSCLGCLREWSSGSQLCEDLLLMPTEGELCEEDCFSNASVKVSYQLQTPEGQTDHPQPILDRYTEPFAIFQLPYEKACKNKLFCVAELQLATTVSQQELVVGLTKELTLNINLTNSGEDSYMTSMALNYPRNLQLKRMQKPPSPNIQCDDPQPVASVLIMNCRIGHPVLKRSSAHVSVVWQLEENAFPNRTADITVTVTNSNERRSLANETHTLQFRHGFVAVLSKPSIMYVNTGQGLSHHKEFLFHVHGENLFGAEYQLQICVPTKLRGLQVVAVKKLTRTQASTVCTWSQERACAYSSVQHVEEWHSVSCVIASDKENVTVAAEISWDHSEELLKDVTELQILGEISFNKSLYEGLNAENHRTKITVVFLKDEKYHSLPIIIKGSVGGLLVLIVILVILFKCGFFKRKYQQLNLESIRKAQLKSENLLEEEN

SEQ ID NO: 42 - protein, 798 aa, Homo sapiens
  REGION 1..798: note = human integrin Beta-7 (amino acid sequence)
MVALPMVLVLLLVLSRGESELDAKIPSTGDATEWRNPHLSMLGSCQPAPSCQKCILSHPSCAWCKQLNFTASGEAEARRCARREELLARGCPLEELEEPRGQQEVLQDQPLSQGARGEGATQLAPQRVRVTLRPGEPQQLQVRFLRAEGYPVDLYYLMDLSYSMKDDLERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGHCHLDSNGLYSRSTEFDYPSVGQVAQALSAANIQPIFAVTSAALPVYQELSKLIPKSAVGELSEDSSNVVQLIMDAYNSLSSTVTLEHSSLPPGVHISYESQCEGPEKREGKAEDRGQCNHVRINQTVTFWVSLQATHCLPEPHLLRLRALGFSEELIVELHTLCDCNCSDTQPQAPHCSDGQGHLQCGVCSCAPGRLGRLCECSVAELSSPDLESGCRAPNGTGPLCSGKGHCQCGRCSCSGQSSGHLCECDDASCERHEGILCGGFGRCQCGVCHCHANRTGRACECSGDMDSCISPEGGLCSGHGRCKCNRCQCLDGYYGALCDQCPGCKTPCERHRDCAECGAFRTGPLATNCSTACAHTNVTLALAPILDDGWCKERTLDNQLFFFLVEDDARGTVVLRVRPQEKGADHTQAIVLGCVGGIVAVGLGLVLAYRLSVEIYDRREYSRFEKEQQQLNWKQDSNPLYKSAITTTINPRFQEADSPTL

SEQ ID NO: 43 - protein, 1178 aa, Macaca mulatta
  REGION 1..1178: note = rhesus integrin Alpha-E (amino acid sequence)
MWLVHTLLCMASLAPLAAFNVDVARPWLTPKGGAPFVLSSLLHQDPGTNHTWLLVTSPRTERTPVPLHRCSLVQDEILCHSVEHVPIPKGRHRGVTVARSHHGVLICIQVLARRPYSLSSEFTGTCGLLGPDLRPQAQANFFDLENLLDPDARVDTGDCYSNKEGSRGEDVNTARRRRALEKEEEEEDEEEEEDEEEEAGTEIAIILDGSGSIDPPDFQRAKDFISNMMRNFYEKCFECNFALVQYGGVIQTEFDLRDSQDVMASLAKVQNITQVGSVTKTASAMQHVLDNIFTSSHGSRRKASKVMVVLTDGGIFEDPLDLTTVINSPKMHGVERFAIGVGEEFKSARTERELNLIASDPDETHAFKVTNYMALDGLLSKLRYNIISMEGTVGDALHYQLAQIGFSAQILDERQVLLGAVGAFDWSGGALLYNTRSRRGRFLNQTAAAVDGEAAQYSYLGYAVAVLHKTCSVSYVAGAPRYKHHGAVFELQKEGTETSFLPVLEGEQMGSYFGSELCPVDIDMDGTTDFLLVAAPFYHVHGEEGRVYVYRLSEQDGSFSLARILSGHPGFASARFGFAMAAVGDISQDKLTDVAIGAPLEGFGAGDGASFGSVYIYNGHWDGLSAGPSQRIRASAVAPGLQYFGMSVAGGFDISGDGLADITVGTLGRAVVFRSRPVVRLEVSMAFTPSALPIGFNGVVNVRLCFEISSVATVSASGLRGAFLNFTLDVDVGKERKRLQCSDGRSCLGCLREWSSGSRLCEDLLLVPTEGELHEEDCFSNATVKVGYQLQTPEGQTDHPQPILDRYAETFAIFQLPYEKACKNKLFCVAELQLATTVSQQELVVGLTKELTLNISLTNSGEDSYMTSMALNYPRNLQFKRMQKPPSPNIQCDDPQPAASVLVMTCRIGHPVLRRSSAHVSVVWQLEENAFPNRTADITVTVTNSNERRSVAEETHTLQFRHGFVAVLSKPSIMYVHTGQVLSHHKEFVFHIHGENLFGAEYQLRICVPTKLRGLQIVTVKNLTRTQAFTVCTWSQERACGFIPVQHVEEWHSVSCVIASDKENVTVAAEISVDHSEELLKDVTELQILGEISFNKSLYEGLNAENHRTKITVVFLKDEKYHSLPVIIKGSIGGLLVLIVILVILFKCGFFKRKYQQLNLENIRKAQLKSETLLEEEN

SEQ ID NO: 44 - protein, 807 aa, Macaca mulatta
  REGION 1..807: note = rhesus integrin Beta-7 (amino acid sequence)
MGFCHVDQGMVALPVVLVLLLVLSRGESELDAKTPSTGEATEWGNPHLSLLGSCQPAPSCQKCIVSHPSCAWCKQLNFTASGEAEARRCARREELLARGCPLEELEEPRGQQEVLQDQPLSQGARGEGATQLAPQRVRITLRPGEPQQLQVRFLRAEGYPVDLYYLMDLSYSMKDDLERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGHCHLDSNGLYSRSTEFDYPSVGQVAQALSAANIQPIFAVTSAALPVYQELSKLIPKSAVGELSEDSSNVVQLIMDAYNSLSSTVTLEHSSLPPGVHISYESQCEGPEKTEGKAEDRGQCNHVQINQTVTFWVSLQATHCLPEPHLLRLRALGFSEELIVELHTLCDCNCSDTQAQAPHCSDGQGHLQCGVCSCAPGRLGRLCECSEAELSSLDLESGCRAPNGTGPLCSGKGQCQCGHCSCNGQSSGHLCECDDASCERHEGILCGGFGRCQCGVCHCHANRTGRACECSGDMDSCISPEGGLCSGHGRCKCNRCQCSDGYYGALCDQCPGCKTPCERHRDCAECGAFGTGLLATNCSTACAHTNVTLVLAPILDDGWCKERTLDNHLFFFLVEDDARGRVVLRVRPQEKGADHTQAIVLGCVGGIVAVGLGLVLAYRLSVEIYDRREYSRFEKEQQQLNWKQDSNPLYKSAITTTINPRFQEADSPIL

SEQ ID NO: 45 - protein, 1032 aa, Homo sapiens [sequence omitted]